(19) United States
(12) Patent Application Publication
(10) Pub. No.: US 2026/0165976 A1
(43) Pub. Date: Jun. 18, 2026

(54) ANTI-EVAPORATIVE LIPID FORMULATIONS

(71) Applicant: UNIVERSITY OF HELSINKI, Helsinki (FI)

(72) Inventors: Filip EKHOLM, Helsinki (FI); Riku PAANANEN, Helsinki (FI); Tuomo VIITAJA, Helsinki (FI); Julia SEVÓN, Helsinki (FI); Jan-Erik RAITANEN, Helsinki (FI); Tatu LAJUNEN, Kupio (FI); Marika RUPONEN, Kupio (FI); Janika JÄNTTI, Kupio (FI)

(73) Assignee: UNIVERSITY OF HELSINKI, Helsinki (FI)

(21) Appl. No.: 19/124,080

(22) PCT Filed: Nov. 1, 2023
(86) PCT No.: PCT/EP2023/080480

§ 371 Date: Apr. 24, 2025

(30) Foreign Application Priority Data
Nov. 2, 2022 (FI) 20225982

(51) Int. Cl. A61K 9/48; A61K 9/00; A61K 9/127

(52) U.S. Cl. CPC A61K 9/4858; A61K 9/0019; A61K 9/0048; A61K 9/127

(57) ABSTRACT

There is provided herein a composition comprising liposomes, which comprise a lipid component and a phospholipid component, which compositions are useful in the treatment of ocular surface disorders.

### FIELD OF INVENTION

The present invention relates to the fields of life sciences and medicine. In more detail, the invention relates to compositions comprising specific evaporation-resistant-lipids and phospholipids. The invention particularly relates to liposomes and liposomal compositions comprising this combination of lipids, as well as the evaporation-resistant lipids per se and the medical use of such lipids and compositions in the treatment and/or prevention of ocular surface diseases, such as dry eye disease and Meibomian gland dysfunction, or alleviation of eye discomfort.

### BACKGROUND OF INVENTION

The listing or discussion of an apparently prior-published document in this specification should not necessarily be taken as an acknowledgement that the document is part of the state of the art or is common general knowledge.

Ocular surface diseases such as dry eye disease (DED) and Meibomian gland dysfunction (MGD) affect 10-30% of the global adult population and constitute a severe societal economic burden with annual managing costs estimated at $55 billion in the United States alone. The incidence of DED is expected to increase significantly in the future due to the close relationship between the risk factors of the disease and modern societal trends (aging population, multiscreen lifestyle, increased use of contact lenses etc.). While DED is the most common reason for seeking medical eye care, the current available treatments on the market are often associated with unsatisfactory results.

The commonly used artificial tears and ocular lubricants alleviate the symptoms of DED but they are unable to efficiently target the central tear film instability defect associated with the disease and need to be applied frequently as a result. Topical corticosteroids, on the other hand, are used to treat the ocular surface inflammation resulting from progressed states of DED but they display severe side-effects in long-term use which is suboptimal considering the chronic nature of some instances of DED. Medical experts have therefore concluded that there is a dire need for improved DED treatments which are capable of efficiently targeting the central tear film instability defect (Jones, L. et al. Ocul. Surf. 2017, 15, 575-628).

The underlying cause of the tear film instability defect associated with DED is a dysfunction in the Meibomian glands which alters the composition of the tear film lipid layer (TFLL). In healthy eyes, the TFLL covers the ocular surface and acts as a biological barrier which protects the underlying epithelial cells from the environment, and, stabilizes the tear film (Brown, S. H. et al. Invest. Ophthalmol. Vis. Sci. 2013, 54, 7417-7423; Lam, S. M. et al. J. Lipid Res. 2014, 55, 299-306; Rohit, A. et al. Optom. Vis. Sci. 2014, 91, 1384-1390).

The TELL is a unique biological membrane mainly consisting of ultra-long non-polar wax esters (WEs) and cholesteryl esters (CEs), with smaller amounts of more polar lipids such as (O-acyl) ω-hydroxy fatty acids (OAHFAs,) their structurally related type-II and type-I-St diester species, and phospholipids also present (Brown, S. H. et al. Invest. Ophthalmol. Vis. Sci. 2013, 54, 7417-7423; Lam, S. M. et al. J. Lipid Res. 2014, 55, 299-306; Rohit, A. et al. Optom. Vis. Sci. 2014, 91, 1384-1390).

In DED patients, the alteration in TFLL structure leads to the partial loss of its capabilities. In more detail, the ability to stabilize the tear film is compromised by a disruption in film properties leading to a more rapid tear film break-up time and a partial loss of TFLL evaporation resistance. This results in an unstable tear film, an increased evaporation rate of aqueous tear fluid and subsequent drying of the eyes (Craig, J., P. et al. Ocul. Surf. 2017, 15, 276-283). Therefore, an ideal treatment solution for DED should be viewed as one able to efficiently combat these defects. While the need for a new effective composition for treating and/or alleviating the symptoms of DED, MGD, and eye discomfort is undeniable, the successful development of functional medicaments displaying such properties represents a significant challenge.

In fact, the development of such a treatment is not a trivial task as it requires considerable insights on the roles of the >200 unique lipid species present in the TFLL. Through successful studies of the major lipid classes existing in the TFLL we have been able to identify promising lipid species for incorporation into new types of treatment strategies targeting the central TFLL destabilizing defects. However, as will be discussed in the contents of this application-identification of promising lipid species in not equivalent to development of a functional medicament for use in treatment of DED/MGD or for alleviating eye discomfort.

Certain lipid species, including particularly certain O-acyl-w-hydroxy fatty acids, including 20-(oleoyloxy) eicosanoic acid and mixtures of this compound with the wax ester behenyl oleate have been found to form strongly evaporation-resistant films when applied to an aqueous surface (Bland T, Langmuir, 2019, 35, 3545-3552; Viitaja et al., Nano Lett, 2021, 21, 7676-7683). However, the development of formulations capable of delivering these species while maintaining useful evaporation resistant properties has proved a significant challenge.

### DETAILED DESCRIPTION OF THE INVENTION

We have now surprisingly found that combining certain evaporation-resistant active ingredients with certain phospholipids into liposomal pharmaceutical formulations, results in evaporation-resistant formulations with promising properties for the treatment of ocular surface disorders or alleviating eye discomfort. In particular, the compositions have promising anti-evaporative properties and display similar biophysical profiles to human Meibum.

In a first aspect of the invention, there is provided a pharmaceutical composition comprising liposomes, which liposomes comprise:
(a) a lipid component selected from the group consisting of:
(i) a fatty acid ester of a hydroxy fatty acid of formula I, or a pharmaceutically acceptable salt thereof,

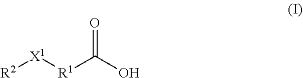

wherein:
R¹ is selected from a C₁₆-C₃₀ alkanediyl or a C₂₁-C₃₀ alkenediyl group containing one double bond;
R² is selected from a C₁₅-C₁₉ alkyl group or a C₁₅-C₁₉ alkenyl group containing one or two double bonds; and
X¹ represents an ester group selected from the group consisting of:

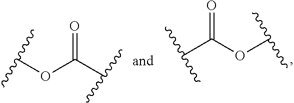

wherein [symbol] represents a point of attachment to the rest of the molecule;
(ii) a combination of a fatty acid ester of a hydroxy fatty acid of formula I, or a pharmaceutically acceptable salt thereof, as defined in (i), and a wax ester of formula II

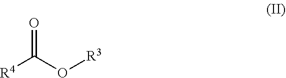

wherein:
R³ represents a C₁₈-C₃₀ linear or branched alkyl group, and
R⁴ represents a C₁₀-C₂₄ linear or branched alkyl group or a C₁₀-C₂₄ linear or branched alkenyl group containing one or two double bonds;
wherein the fatty acid ester of a hydroxy fatty acid of formula I and the wax ester of formula II are present in a molar ratio of from about 2:1 to about 1:9 (fatty acid ester of a hydroxy fatty acid:wax ester);
(iii) a fatty alcohol of formula III, or a pharmaceutically acceptable salt thereof,

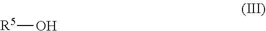

wherein R⁵ represents a C₁₄-C₃₀ linear or branched alkyl group; or formula IV,

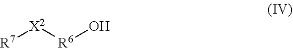

wherein:
R⁶ is selected from a C₁₀-C₃₀ alkanediyl or a C₁₀-C₃₀ alkenediyl group containing one double bond;
R⁷ is selected from a C₁₄-C₁₉ alkyl group or a C₁₄-C₁₉ alkenyl group containing one or two double bonds; and
X² represents an ester group selected from the group consisting of

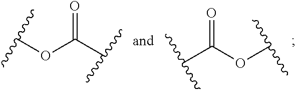 [SMILES: *OC(*)=O ; *OC(*)=O]

(iv) a combination of a fatty acid ester of a hydroxy fatty acid of formula I, or pharmaceutically-acceptable salt thereof with a fatty alcohol of formula III or IV, or a pharmaceutically acceptable salt thereof, wherein the components are present in an amount of from about 3:1 to about 1:3; or
(v) a combination of a wax ester of formula II with a fatty alcohol of formula III or IV, or a pharmaceutically acceptable salt thereof, wherein the components are present in an amount of from about 3:1 to about 1:3; and
(b) a phospholipid component, which is selected from the group consisting of a phosphatidylcholine, a phosphatidylglycerol, or a pharmaceutically acceptable salt thereof, a phosphatidylserine, or a pharmaceutically acceptable salt thereof, a phosphatidic acid, or a pharmaceutically acceptable salt thereof, a phosphatidylethanolamine and a mixture thereof;
wherein the lipid component and phospholipid components are present, in the composition, in a ratio from about 10:1 to about 1:10 by mass, such compositions may be referred to herein as ‘the compositions of the invention’.

For the avoidance of doubt, the skilled person will understand that references herein to compounds of particular aspects of the invention (such as the first aspect of the invention, i.e. referring to compounds of formula I as defined in the first aspect of the invention) will include references to all embodiments and particular features thereof, which embodiments and particular features may be taken in combination to form further embodiments and features of the invention.

Unless indicated otherwise, all technical and scientific terms used herein will have their common meaning as understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalene-disulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

In particular, salts of fatty acid esters of hydroxy fatty acids (including, particularly, (O-acyl) w-hydroxy fatty acids (OAHFAs)) may be base addition salts. Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and Inorganic bases (such as ammonia and aluminium hydroxide). More particularly, base addition salts that may be mentioned include Mg, Ca and, most particularly, K and Na salts.

In particular embodiments, the lipid and phospholipid components of the compositions and liposomes described herein are present in their non-salt forms (i.e. as the free acid or base). References to ratios, and amounts, of the different components in the liposomes and compositions described herein may particularly refer to the ratios between the non-salt species.

The lipid and phospholipid components of the liposomes and compositions described herein may contain double bonds and, unless otherwise indicated, may thus exist as E (entgegen) and Z (zusammen) geometric isomers about each individual double bond. Unless otherwise specified, all such isomers and mixtures thereof are included within the scope of the invention.

The lipid and phospholipid components of the liposomes and compositions described herein may also exhibit tautomerism. All tautomeric forms and mixtures thereof are included within the scope of the invention (particularly those of sufficient stability to allow for isolation thereof).

The lipid and phospholipid components of the liposomes and compositions described herein may also contain one or more asymmetric carbon atoms and may therefore exhibit optical and/or diastereoisomerism (i.e. existing in enantiomeric or diastereomeric forms). Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired enantiomer or diastereoisomer may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution; for example, with a homochiral acid followed by separation of the diastereomeric derivatives by conventional means such as chromatography), or by reaction with an appropriate chiral reagent or chiral catalyst, all of which methods and processes may be performed under conditions known to the skilled person. Unless otherwise specified, all stereoisomers and mixtures thereof are included within the scope of the invention.

Unless otherwise specified, C_{1-z} alkyl groups (where z is the upper limit of the range) defined herein may be straight-chain or, when there is a sufficient number (i.e. a minimum of two or three, as appropriate) of carbon atoms, be branched-chain. For the avoidance of doubt, particular alkyl groups that may be mentioned include straight chain (linear) (i.e. not branched and/or cyclic) alkyl groups. References to alkyl groups may generally be understood to refer to a univalent group, such as a group derived from an alkane by the removal of a hydrogen atom.

Unless otherwise specified, C_{1-z} alkanediyl groups (where z is the upper limit of the range) defined herein may be straight-chain or, when there is a sufficient number (i.e. a minimum of two or three, as appropriate) of carbon atoms, be branched-chain. For the avoidance of doubt, particular alkanediyl groups that may be mentioned include straight chain (linear) (i.e. not branched and/or cyclic) alkyl groups. References to alkanediyl groups may be understood to refer to a divalent group, such as a group derived from an alkane by the removal of two hydrogen atoms. In particular, alkanediyl groups refer to divalent groups in which the points of attachment of the group to the rest of the molecule are positioned at each end of the alkyl chain, or, in the case of branched alkanediyl groups, at each end of the longest alkyl chain in the group.

Unless otherwise specified, C_{2-z} alkenyl groups (where z is the upper limit of the range) defined herein may be straight-chain or, when there is a sufficient number (i.e. a minimum of three) of carbon atoms, be branched-chain. For the avoidance of doubt, particular alkenyl groups that may be mentioned include straight chain (linear) (i.e. not branched and/or cyclic) alkenyl groups. References to alkenyl groups may generally be understood to refer to a univalent group, such as a group derived from an alkene by the removal of a hydrogen atom.

Unless otherwise specified, C_{1-z} alkenediyl groups (where z is the upper limit of the range) defined herein may be straight-chain or, when there is a sufficient number (i.e. a minimum of two or three, as appropriate) of carbon atoms, be branched-chain. For the avoidance of doubt, particular alkenediyl groups that may be mentioned include straight chain (linear) (i.e. not branched and/or cyclic) alkendiyl groups. References to alkenediyl groups may be understood to refer to a divalent group, such as a group derived from an alkene by the removal of two hydrogen atoms. In particular, alkenediyl groups refer to divalent groups in which the points of attachment of the group to the rest of the molecule are positioned at each end of the alkenyl chain, or, in the case of branched alkanediyl groups, at each end of the longest alkenyl chain in the group.

As used herein, the term ‘liposome’ may be understood to refer to a spherical artificial vesicle comprising at least one lipid bilayer (in particular, the liposomes described herein will contain a lipid component and a phospholipid component as defined herein (and present in the ratios described herein). The skilled person will understand that such species are generally present in aqueous solution.

Particular lipid species used in the compositions and liposomes described herein are shown in the table below.

| | |
|---|---|
| Structure and name | Abbreviation(s) |

(O-acryl) ω-hydroxy fatty acids (OAHFas)

| | |
|---|---|
| 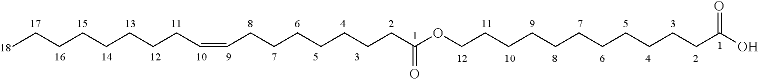 [SMILES: CCCCCCCC/C=C\CCCCCCCC(=O)OCCCCCCCCCCCC(=O)O] | 12:0/18:1-OAHFA |
| 12-(oleoyloxy)dodecanoic acid | |
| 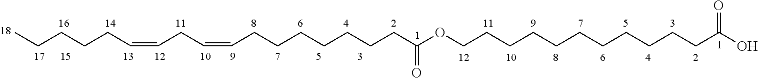 [SMILES: CCCCC/C=C\C/C=C\CCCCCCCC(=O)OCCCCCCCCCCCC(=O)O] | 12:0/18:2-OAHFA |
| 12-(linoleoyloxy)dodecanoic acid | |
| 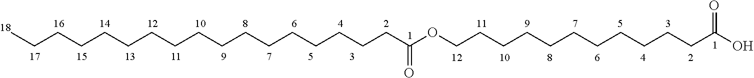 [SMILES: CCCCCCCCCCCCCCCCCC(=O)OCCCCCCCCCCCC(=O)O] | 12:0/18:0-OAHFA |
| 12-(stearoyloxy)dodecanoic acid | |
| 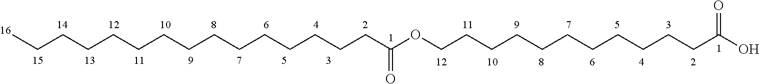 [SMILES: CCCCCCCCCCCCCCCC(=O)OCCCCCCCCCCCC(=O)O] | 12:0/16:0-OAHFA |
| 12-(palmitoyloxy)dodecanoic acid | |
| 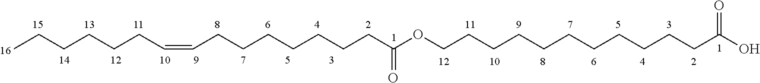 [SMILES: CCCCCC/C=C\CCCCCCCC(=O)OCCCCCCCCCCCC(=O)O] | 12:0/16:1-OAHFA |
| 12-(palmitoleoyloxy)dodecanoic acid | |
| 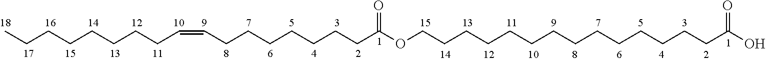 [SMILES: CCCCCCCC/C=C\CCCCCCCC(=O)OCCCCCCCCCCCCCCC(=O)O] | 15:0/18:1-OAHFA |
| 15-(oleoyloxy)pentadecanoic acid | |
| 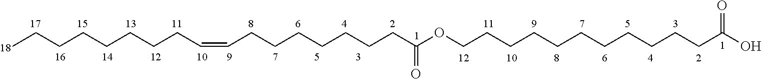 [SMILES: CCCCCCCC/C=C\CCCCCCCC(=O)OCCCCCCCCCCCC(=O)O] | 18:0/18:1-OAHFA; 18-OAHFA |
| 18-(oleoyloxy)octadecanoic acid | |
| 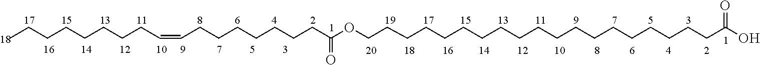 [SMILES: CCCCCCCC/C=C\CCCCCCCC(=O)OCCCCCCCCCCCCCCCCCCCC(=O)O] | 20:0/18:1-OAHFA; 20-OAHFA |
| 20-(oleoyloxy)eicosanoic acid | |
| 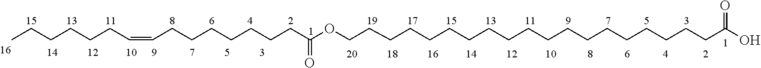 [SMILES: CCCCCC/C=C\CCCCCCCC(=O)OCCCCCCCCCCCCCCCCCCCC(=O)O] | 20:0/16:1-OAHFA |
| 20-(palmitoleoyloxy)eicosanoic acid | |
| 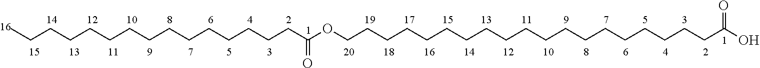 [SMILES: CCCCCCCCCCCCCCCC(=O)OCCCCCCCCCCCCCCCCCCCC(=O)O] | 20:0/16:0-OAHFA |
| 20-(palmitoyloxy)eicosanoic acid | |
| 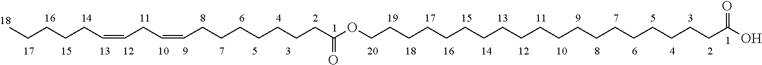 [SMILES: CCCCC/C=C\C/C=C\CCCCCCCC(=O)OCCCCCCCCCCCCCCCCCCCC(=O)O] | 20:0/16:2-OAHFA |
| 20-(linoleoyloxy)eicosanoic acid | |
| 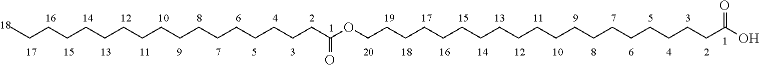 [SMILES: CCCCCCCCCCCCCCCCCC(=O)OCCCCCCCCCCCCCCCCCCCC(=O)O] | 20:0/18:0-OAHFA |
| 20-(stearoyloxy)eicosanoic acid | |
| 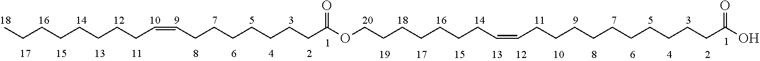 [SMILES: CCCCCCCC/C=C\CCCCCCCC(=O)OCCCCCCC/C=C\CCCCCCCCCCC(=O)O] | 20:1/18:1-OAHFA |
| (12Z)-20-(oleoyloxy)eicos-12-enoic acid | |
| 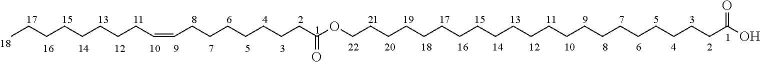 [SMILES: CCCCCCCC/C=C\CCCCCCCC(=O)OCCCCCCCCCCCCCCCCCCCCCC(=O)O] | 22:0/18:1-OAHFA |
| 22-(oleoyloxy)docosanoic acid | |
| 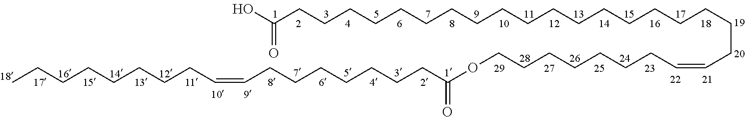 [SMILES: CCCCCCCC/C=C\CCCCCCCC(=O)OCCCCCCC/C=C\CCCCCCCCCCCCCCCCCCCC(=O)O] | 29:1/18:1-OAHFA |
| (21Z)-29-(oleoyloxy)nonacos-21-enoic acid | |

Reversed-OAHFAs

| | |
|---|---|
| 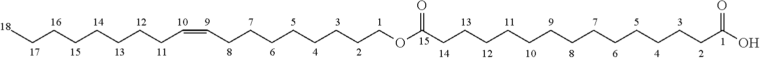 [SMILES: CCCCCCCC/C=C\CCCCCCCCOC(=O)CCCCCCCCCCCCCC(=O)O] | R-15:0/18:1-OAHFA |
| 15-(oleyloxy)-15-oxo-pentadecanoic acid | |
| 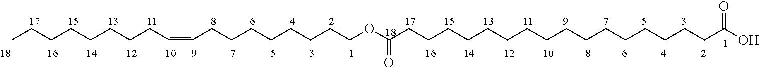 [SMILES: CCCCCCCC/C=C\CCCCCCCCOC(=O)CCCCCCCCCCCCCCCCC(=O)O] | R-18:0/18:1-OAHFA |
| 18-(oleyloxy)-18-oxo-octadecanoic acid | |
| 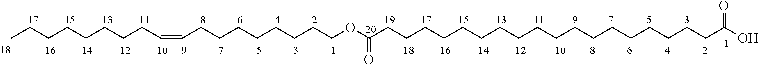 [SMILES: CCCCCCCC/C=C\CCCCCCCCOC(=O)CCCCCCCCCCCCCCCCCCC(=O)O] | R-20:0/18:1-OAHFA |
| 20-(oleyloxy)-20-oxo-eicosanoic acid | |

Wax esters

| | |
|---|---|
| 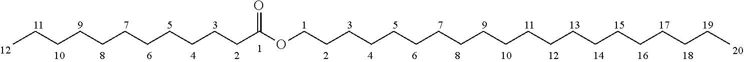 [SMILES: CCCCCCCCCCCCCCCCCCCCOC(=O)CCCCCCCCCCC] | AL |
| arachidyl laurate | |
| 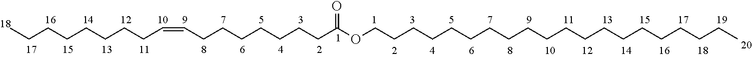 [SMILES: CCCCCCCC/C=C\CCCCCCCC(=O)OCCCCCCCCCCCCCCCCCCCC] | AO |
| arachidyl oleate | |
| 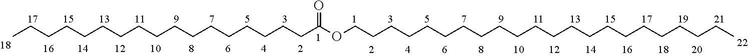 [SMILES: CCCCCCCCCCCCCCCCCCCCCCOC(=O)CCCCCCCCCCCCCCCCC] | BS |
| behenyl stearate | |
| 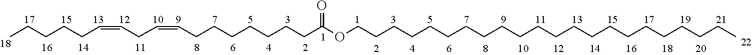 [SMILES: CCCCC/C=C\C/C=C\CCCCCCCC(=O)OCCCCCCCCCCCCCCCCCCCCCC] | BLN |
| behenyl linoleate | |
| 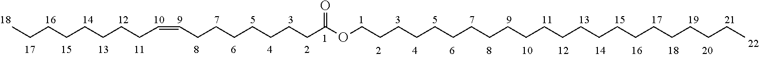 [SMILES: CCCCCCCC/C=C\CCCCCCCC(=O)OCCCCCCCCCCCCCCCCCCCCCC] | BO |
| behenyl oleate | |
| 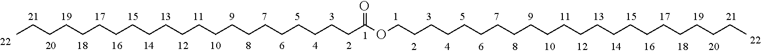 [SMILES: CCCCCCCCCCCCCCCCCCCCCCOC(=O)CCCCCCCCCCCCCCCCCCCCC] | BB |
| behenyl behenate | |
| 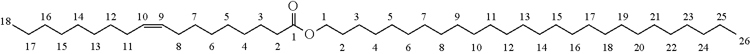 [SMILES: CCCCCCCC/C=C\CCCCCCCC(=O)OCCCCCCCCCCCCCCCCCCCCCCCCCC] | HO |
| hexacosyl oleate | |
| 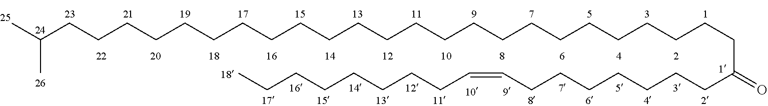 [SMILES: CCCCCCCC/C=C\CCCCCCCC(=O)CCCCCCCCCCCCCCCCCCCCCCCCC(C)C] | iso-WE |
| 24-methylpentacosyl oleate | |

Long chain fatty alchols

| | |
|---|---|
| 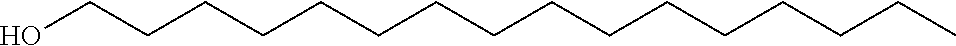 [SMILES: CCCCCCCCCCCCCCCCO] | |
| hexadecanol | |
| 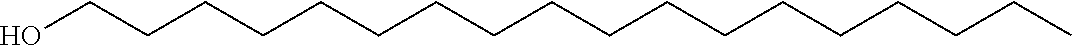 [SMILES: CCCCCCCCCCCCCCCCCCO] | |
| octadecanol | |
| 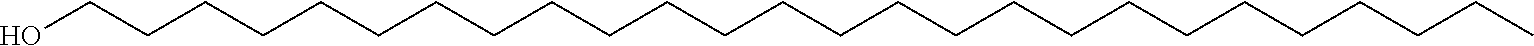 [SMILES: CCCCCCCCCCCCCCCCCCCCCCCCCCO] | |
| hexacosanol | |
| 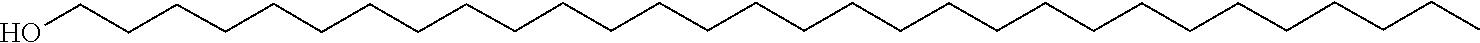 [SMILES: CCCCCCCCCCCCCCCCCCCCCCCCCCCCCCO] | |
| triacontanol | |
| 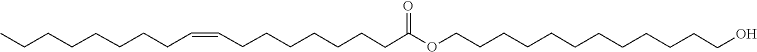 [SMILES: CCCCCCCC/C=C\CCCCCCCC(=O)OCCCCCCCCCCCCO] | 12:0/18:1-OAHFAI |
| 12-(oleoyloxy)dodecanol | |
| 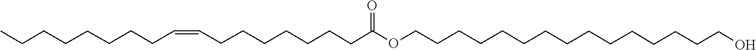 [SMILES: CCCCCCCC/C=C\CCCCCCCC(=O)OCCCCCCCCCCCCCCCO] | 15:0/18:1-OAHFAI |
| 15-(oleoyloxy)pentadecanol | |
| 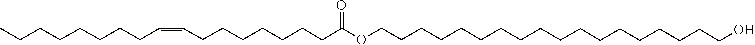 [SMILES: CCCCCCCC/C=C\CCCCCCCC(=O)OCCCCCCCCCCCCCCCCCCO] | 18:0/18:1-OAHFAI |
| 18-(oleoyloxy)octadecanol | |
| 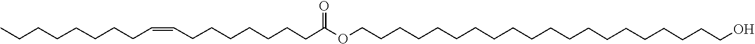 [SMILES: CCCCCCCC/C=C\CCCCCCCC(=O)OCCCCCCCCCCCCCCCCCCCCO] | 20:0/18:1-OAHFAI |
| 20-(oleoyloxy)eicosanol | |

In particular embodiments, that may be mentioned, in the fatty acid ester of the hydroxy fatty acid, R¹ is selected from the group consisting of a C₁₇-C₂₄ alkanediyl group or a C₂₄-C₂₉ alkenediyl group containing one double bond. More particularly, R¹ a C₁₇-C₂₂ alkanediyl group, such as a C₁₇-C₁₉ alkanediyl group or a C₂₇-C₂₉ alkenediyl, such as a C₂₈ alkenediyl group containing one double bond (for example a Z-double bond, which may preferably at the 21 carbon from the carboxylic acid moiety (inclusive of the carbonyl group carbon). Yet more particularly, R¹ is a C₁₇-C₁₉ alkanediyl group (e.g. C₁₇).

In further particular embodiments, X¹ represents

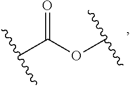 [SMILES: *OC(*)=O]

and/or (e.g. and) R² represents a C₁₅ to C₁₇ alkenyl group containing one or two (e.g. one) double bonds.

In further particular embodiments, X¹ represents

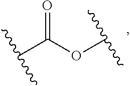 [SMILES: *OC(*)=O]

and/or (e.g. and) R² represents a C₁₅ or C₁₇ alkenyl group containing one or two (e.g. one) double bonds, in particular R² represents

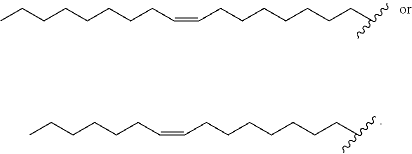 [SMILES: *CCCCCCC/C=C\CCCCCCCC ; *CCCCCCC/C=C\CCCCCC]

In further particular embodiments, X¹ represents

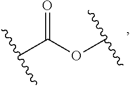 [SMILES: *OC(*)=O]

and/or (e.g. and) R² represents a C₁₇ alkenyl group containing one or two double bonds. More particularly, R² represents

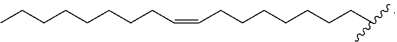 [SMILES: *CCCCCCC/C=C\CCCCCCCC]

In further particular embodiments, X¹ represents

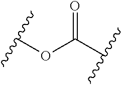 [SMILES: *OC(*)=O]

and/or (e.g. and) R² represents a C₁₆ to C₁₈ alkenyl group containing one or two (e.g. one) double bonds,

In further particular embodiments, X¹ represents

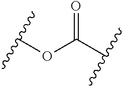 [SMILES: *OC(*)=O]

and/or (e.g. and) R² represents a C₁₆ or C₁₈ alkenyl group containing one or two (e.g. one) double bonds, preferably wherein R² represents

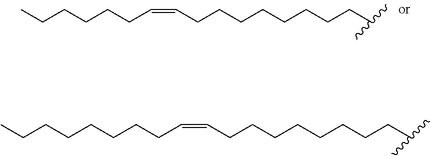 [SMILES: *CCCCCCCC/C=C\CCCCCC ; *CCCCCCCC/C=C\CCCCCCCC]

In further particular embodiments, X¹ represents

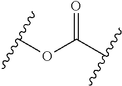 [SMILES: *OC(*)=O]

and/or (e.g. and) R² represents a C₁₈ alkenyl group containing one or two double bonds, preferably wherein R² represents

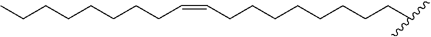 [SMILES: *CCCCCCCC/C=C\CCCCCCCC]

Fatty acid esters of hydroxy fatty acids of formula I in which X¹ represents

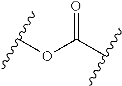 [SMILES: *OC(*)=O]

(i.e. those in which the ester linkage is in the opposite orientation to the most common naturally-occurring variants of such compounds) may be advantageous to use as they surprisingly have similar physical properties but their chemical synthesis can be more straightforward.

In further particular embodiments, R³ represents a C₁₈ to C₂₆ alkyl group. More particularly, R² represents a C₁₈ to C₂₂ alkyl group.

In further particular embodiments, R⁴ represents a C₁₁ to C₂₂ alkyl or a C₁₁ to C₂₂ alkenyl group containing one or two double bonds.

In further particular embodiments, R⁵ represents a C₁₆ to C₂₆ alkyl group. More particularly, R⁵ represents a Cis alkyl group.

In further particular embodiments, X² represents

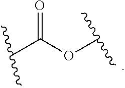 [SMILES: *OC(*)=O]

In further particular embodiments, R⁶ represents a C₁₅-C₂₂ alkanediyl group. More particularly R⁶ represents a C₁₈ to C₂₀ alkanediyl group.

In further particular embodiments, wherein R⁷ represents a C₁₇ or C₁₈ alkenyl group containing one double bond.

In more particular embodiments, X² represents

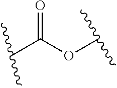 [SMILES: *OC(*)=O]

and R⁷ represents

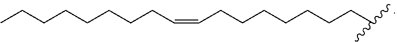 [SMILES: *CCCCCCC/C=C\CCCCCCCC]

In further particular embodiments, the fatty acid ester of a hydroxy fatty acid of formula I (which may, optionally, be present in the form of a pharmaceutically acceptable salt) is selected from the group consisting of:

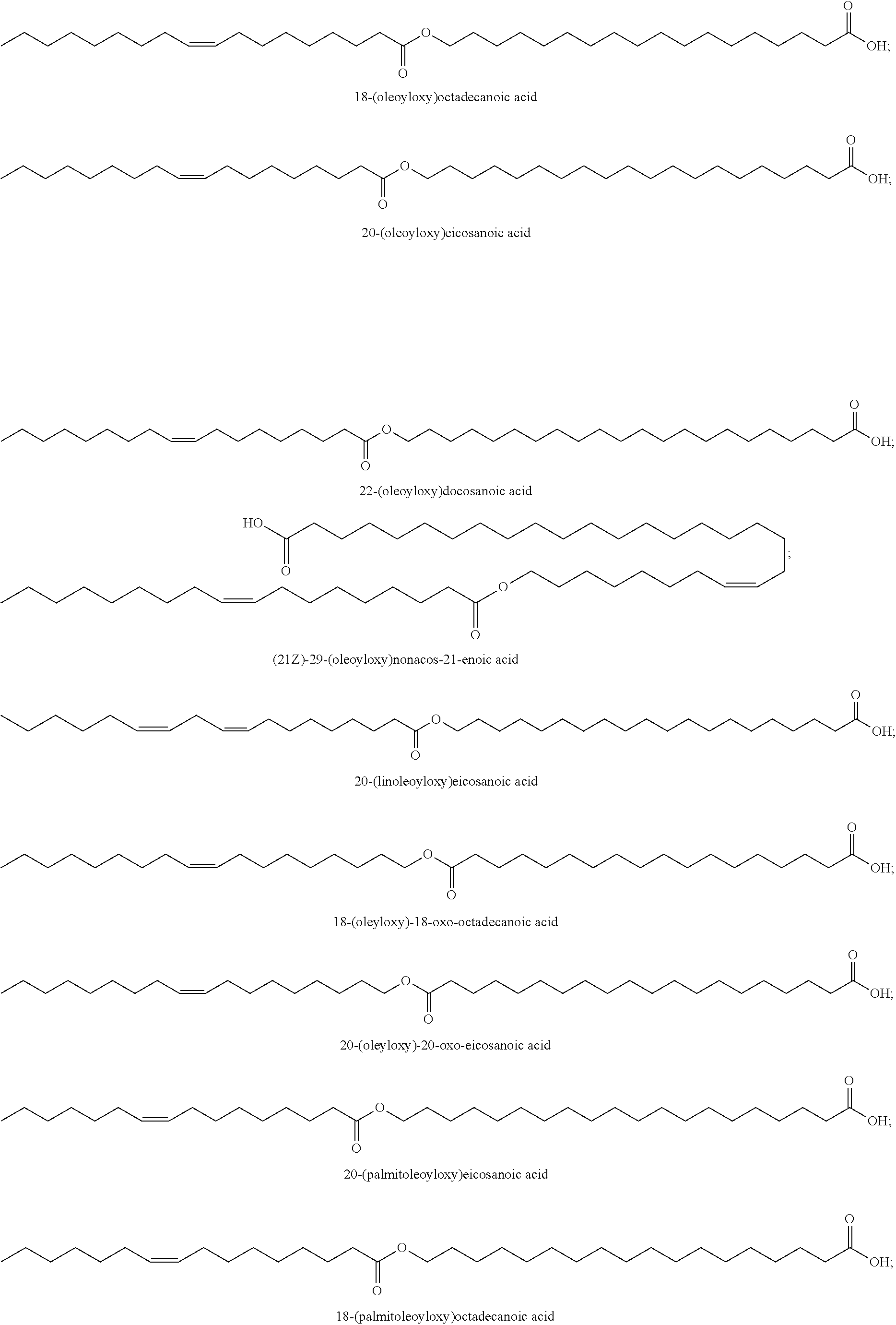

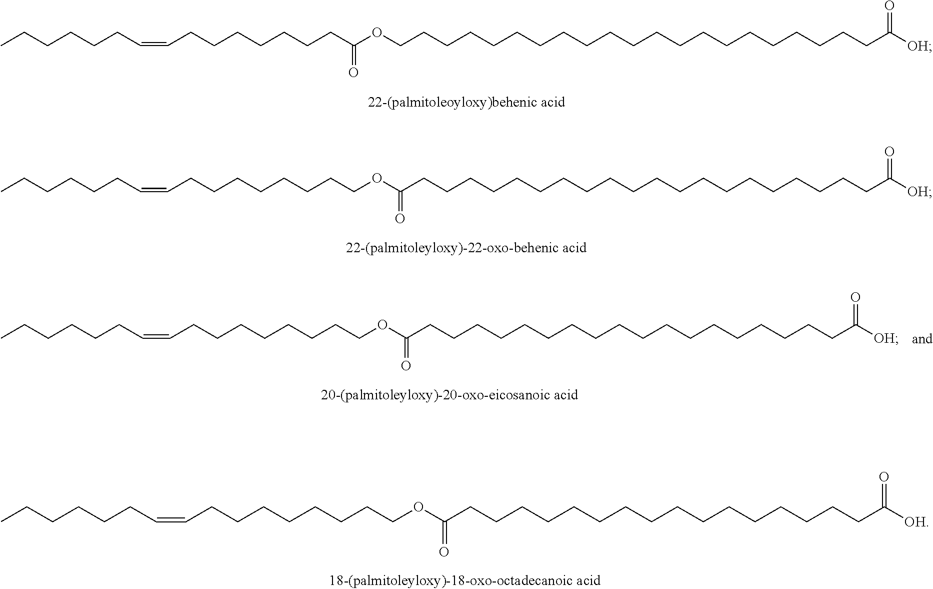 [SMILES: CCCCCC/C=C\CCCCCCCC(=O)OCCCCCCCCCCCCCCCCCCCCCC(=O)O ; CCCCCC/C=C\CCCCCCCCOC(=O)CCCCCCCCCCCCCCCCCCCCC(=O)O ; CCCCCC/C=C\CCCCCCCCOC(=O)CCCCCCCCCCCCCCCCCCC(=O)O ; CCCCCC/C=C\CCCCCCCCOC(=O)CCCCCCCCCCCCCCCCC(=O)O]

In further particular embodiments, the fatty acid ester of a hydroxy fatty acid of formula I (which may, optionally, be present in the form of a pharmaceutically acceptable salt) is selected from the group consisting of:

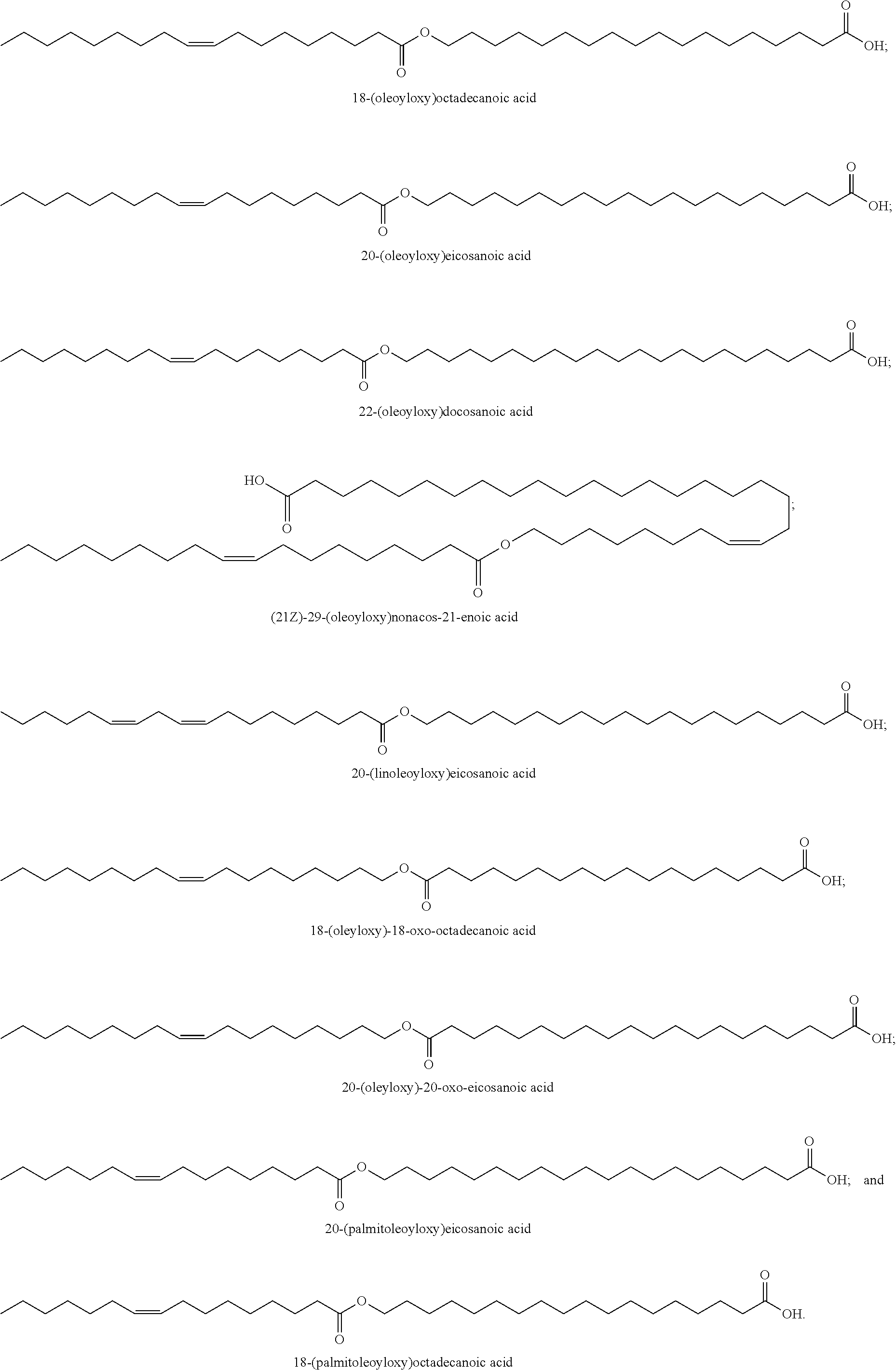

In further particular embodiments, the fatty acid ester of a hydroxy fatty acid of formula I (which may, optionally, be present in the form of a pharmaceutically acceptable salt) is selected from the group consisting of:

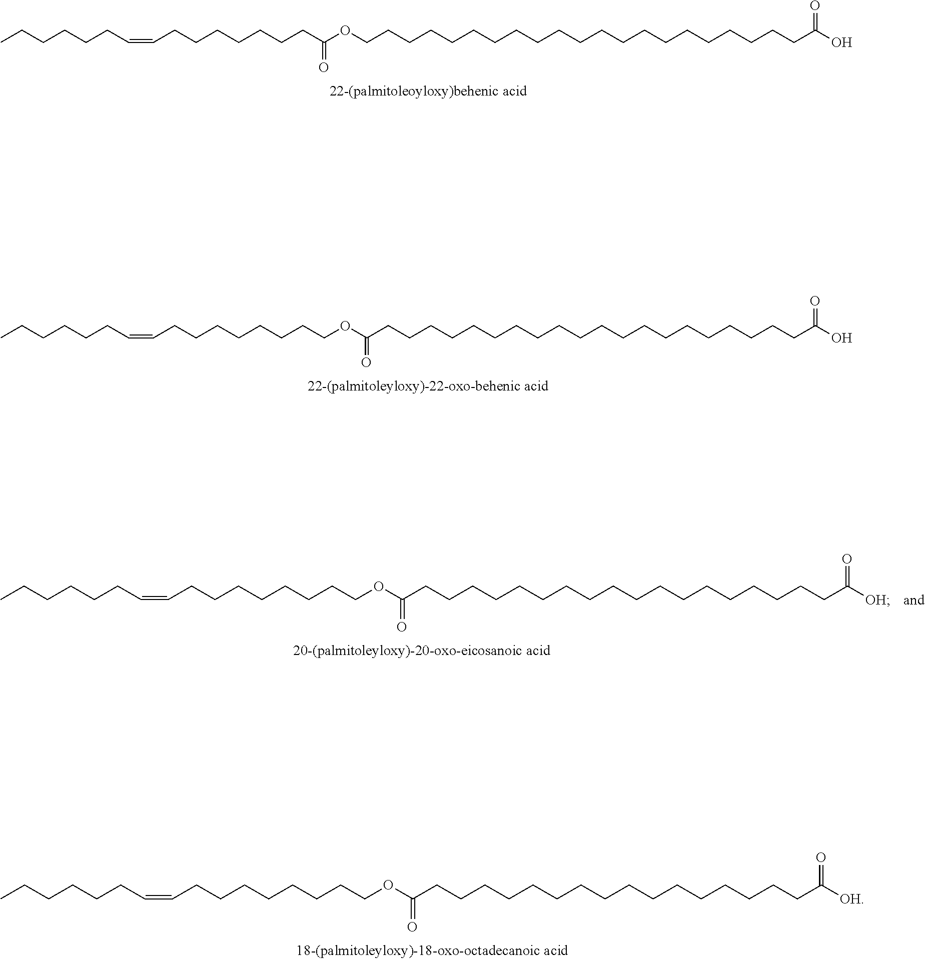 [SMILES: CCCCCC/C=C\CCCCCCCC(=O)OCCCCCCCCCCCCCCCCCCCCCC(=O)O ; CCCCCC/C=C\CCCCCCCCOC(=O)CCCCCCCCCCCCCCCCCCCCC(=O)O ; CCCCCC/C=C\CCCCCCCCOC(=O)CCCCCCCCCCCCCCCCCCC(=O)O ; CCCCCC/C=C\CCCCCCCCOC(=O)CCCCCCCCCCCCCCCCC(=O)O]

In further particular embodiments, the the fatty acid ester of a hydroxy fatty acid of formula I (which may, optionally, be present in the form of a pharmaceutically acceptable salt) is selected from the group consisting of:

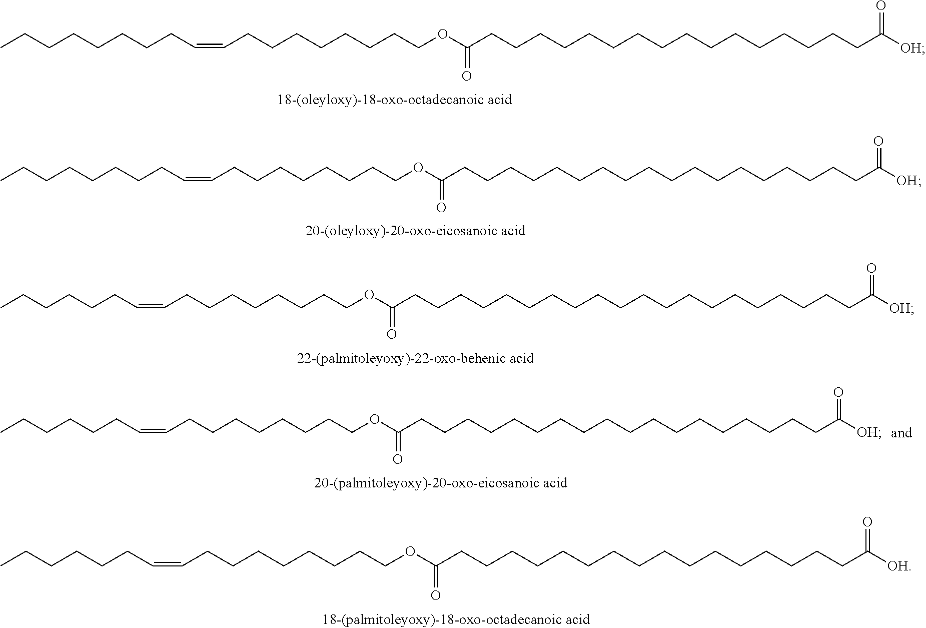 [SMILES: CCCCCCCC/C=C\CCCCCCCCOC(=O)CCCCCCCCCCCCCCCCC(=O)O ; CCCCCCCC/C=C\CCCCCCCCOC(=O)CCCCCCCCCCCCCCCCCCC(=O)O ; CCCCCC/C=C\CCCCCCCCOC(=O)CCCCCCCCCCCCCCCCCCCCC(=O)O ; CCCCCC/C=C\CCCCCCCCOC(=O)CCCCCCCCCCCCCCCCCCC(=O)O ; "and" ; CCCCCC/C=C\CCCCCCCCOC(=O)CCCCCCCCCCCCCCCCC(=O)O]

In yet more particular embodiments, the fatty acid ester of a hydroxy fatty acid of formula I (which may, optionally, be present in the form of a pharmaceutically acceptable salt) is selected from the group consisting of:

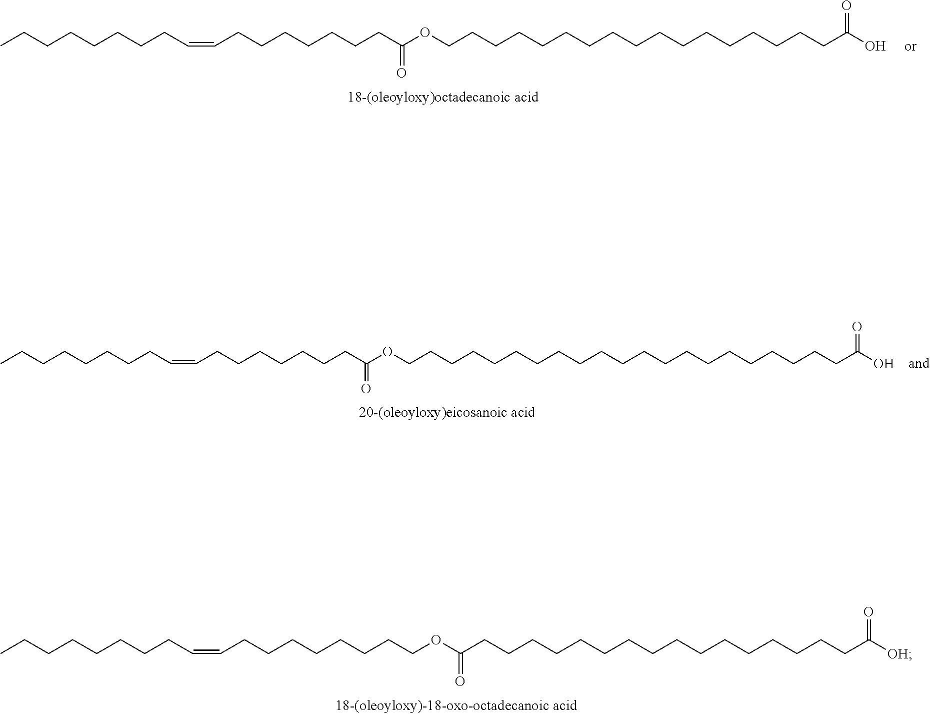 [SMILES: CCCCCCCC/C=C\CCCCCCCC(=O)OCCCCCCCCCCCCCCCCCC(=O)O ; CCCCCCCC/C=C\CCCCCCCC(=O)OCCCCCCCCCCCCCCCCCCCCCC(=O)O ; CCCCCCCC/C=C\CCCCCCCCOC(=O)CCCCCCCCCCCCCCCCC(=O)O]

A particular fatty acid ester of a hydroxy fatty acid that may be mentioned for inclusion in the liposomes and compositions (optionally in the form of a pharmaceutically acceptable salt) described herein is

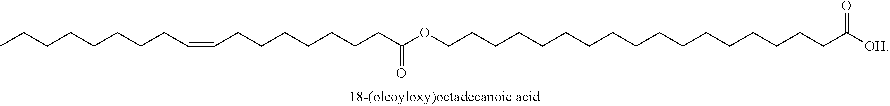 [SMILES: CCCCCCCC/C=C\CCCCCCCC(=O)OCCCCCCCCCCCCCCCCCC(=O)O]

In further particular embodiments that may be mentioned, the wax ester of formula II is selected from the group consisting of:

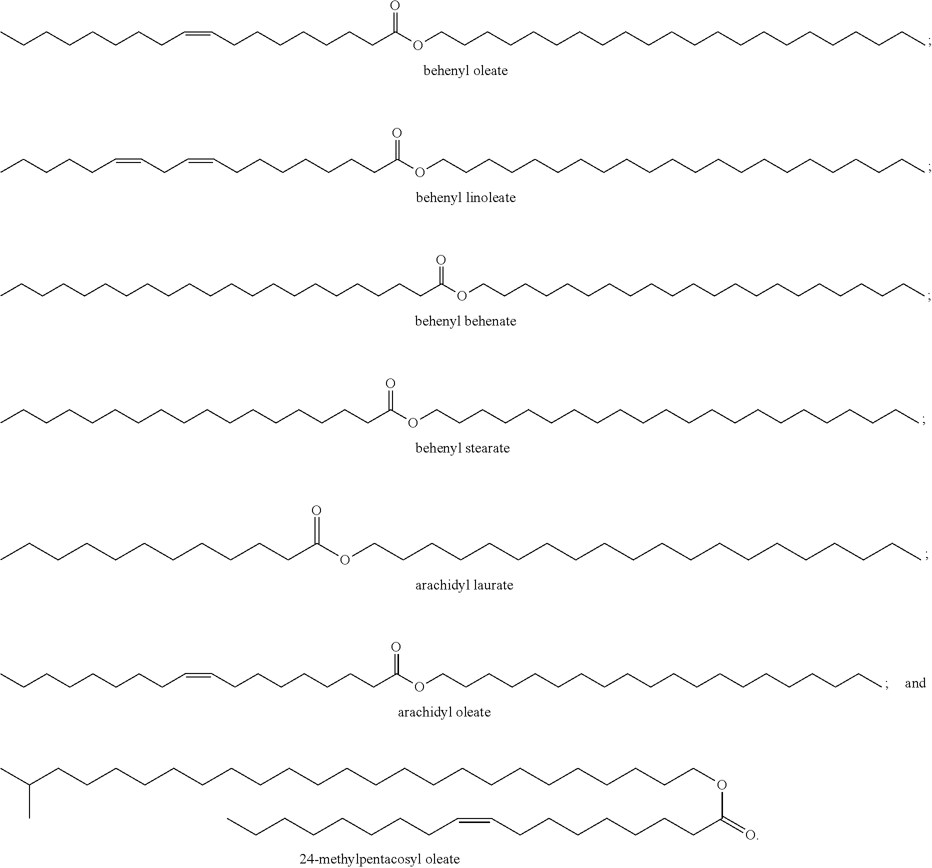

Yet more particularly, the wax ester of formula II is behenyl oleate.

In further particular embodiments, the fatty alcohol included in the liposomes and formulations described herein is of formula III and is octadecanol (which may optionally be present in the form of a pharmaceutically acceptable salt).

In particular embodiments that may be mentioned, the lipid component of the liposomes and compositions described herein is a fatty acid ester of a hydroxy fatty acid of formula I, or a pharmaceutically acceptable salt thereof, as defined herein.

It has previously been reported that, in certain molar ratios, certain fatty acid esters of hydroxy fatty acids and wax esters form highly evaporation resistant films (Viitaja et al., Nano Lett, 2021, 21, 7676-7683; PCT/EP2022/061585). In particular, it was found that combining these components in at least 1:1 molar ratio (fatty acid esters of hydroxy fatty acids:wax esters) resulted in a highly evaporation resistant combined monolayer, increasing the amount of wax ester (up to molar rations of 1:9) also resulted in high evaporation resistance, but the excess wax ester was not incorporated into the monolayer.

Accordingly, in particular embodiments that may be mentioned, the lipid component of the liposomes and compositions described herein is a combination of a fatty acid ester of a hydroxy fatty acid of formula I, or a pharmaceutically acceptable salt thereof, and a wax ester of formula II, wherein the fatty acid ester of a hydroxy fatty acid of formula I and the wax ester of formula II are present in a molar ratio of from about 1:1 to about 1:9 (fatty acid ester of a hydroxy fatty acid:wax ester), for example from about 1:1 to about 1:5, such as from about 1:1 to about 1:3 (e.g. from about 1:1 to about 1:2). More particularly, the fatty acid ester of a hydroxy fatty acid and wax ester are present in a molar ratio of about 1:1.

In general, the mass of the fatty acid ester of a hydroxy fatty acid of formula I and a wax ester of formula II are relatively similar, so the mass ratio and molar ratio will essentially correspond to each other. Thus, in further embodiments, the lipid component of the liposomes and compositions described herein is a combination of a fatty acid ester of a hydroxy fatty acid of formula I and a wax ester of formula II, wherein the fatty acid ester of a hydroxy fatty acid of formula I and the wax ester of formula II are present in a mass ratio of from about 2:1 to about 1:9 (fatty acid ester of a hydroxy fatty acid:wax ester), for example from about 1:1 to about 1:9, such as from about 1:1 to about 1:5 (e.g. from about 1:1 to about 1:3 or about 1:1 to about 1:2). More particularly, the fatty acid ester of a hydroxy fatty acid and wax ester are present in a mass ratio of the fatty acid ester of a hydroxy fatty acid and a wax ester is about 1:1.

In particular embodiments of such liposomes and formulations, the lipid component is selected from the group consisting of
a combination of 20-(oleoyloxy) eicosanoic acid, or a pharmaceutically acceptable salt thereof, and behenyl oleate;
a combination of 20-(oleoyloxy) eicosanoic acid, or a pharmaceutically acceptable salt thereof, and arachidyl laurate;
a combination of 18-(oleoyloxy) octadecanoic acid, or a pharmaceutically acceptable salt thereof, and behenyl oleate;
a combination of 18-(oleoyloxy) octadecanoic acid, or a pharmaceutically acceptable salt thereof, and behenyl linoleate;
a combination of 18-(oleoyloxy) octadecanoic acid, or a pharmaceutically acceptable salt thereof, and behenyl behenate;
a combination of 18-(oleoyloxy) octadecanoic acid, or a pharmaceutically acceptable salt thereof, and behenyl stearate;
a combination of 18-(oleoyloxy) octadecanoic acid, or a pharmaceutically acceptable salt threof, and arachidyl laurate;
a combination of 18-(oleyloxy)-18-oxo-octadecanoic acid, or a pharmaceutically acceptable salt thereof, and behenyl oleate;
a combination of (21Z)-29-(oleoyloxy) nonacos-21-enoic acid, or a pharmaceutically acceptable salt thereof, and 24-methylpentacosyl oleate;
wherein, for each combination, the fatty acid ester of a hydroxy fatty acid and wax ester are present in a molar ratio of about 1:1 to about 1:9 (fatty acid ester of a hydroxy fatty acid:wax ester), for example from about 1:1 to about 1:5, such as from about 1:1 to about 1:3 (e.g. from about 1:1 to about 1:2). More particularly, the fatty acid ester of a hydroxy fatty acid and wax ester are present in a molar ratio of about 1:1. Such lipid components may also contain the fatty acid ester of a hydroxy fatty acid and wax ester in the corresponding mass ratios.

In particular embodiments of such liposomes and formulations, the lipid component is selected from the group consisting of
a combination of 20-(oleoyloxy) eicosanoic acid and behenyl oleate;
a combination of 20-(oleoyloxy) eicosanoic acid and arachidyl laurate;
a combination of 18-(oleoyloxy) octadecanoic acid and behenyl oleate;
a combination of 18-(oleoyloxy) octadecanoic acid and behenyl linoleate;
a combination of 18-(oleoyloxy) octadecanoic acid and behenyl behenate;
a combination of 18-(oleoyloxy) octadecanoic acid and behenyl stearate;
a combination of 18-(oleoyloxy) octadecanoic acid and arachidyl laurate;
a combination of 18-(oleyloxy)-18-oxo-octadecanoic acid and behenyl oleate;
a combination of (21Z)-29-(oleoyloxy) nonacos-21-enoic acid and 24-methylpentacosyl oleate;
wherein, for each combination, the fatty acid ester of a hydroxy fatty acid and wax ester are present in a molar ratio of about 1:1 to about 1:9 (fatty acid ester of a hydroxy fatty acid:wax ester), for example from about 1:1 to about 1:5, such as from about 1:1 to about 1:3 (e.g. from about 1:1 to about 1:2). More particularly, the fatty acid ester of a hydroxy fatty acid and wax ester are present in a molar ratio of about 1:1. Such lipid components may also contain the fatty acid ester of a hydroxy fatty acid and wax ester in the corresponding mass ratios.

More particularly, the lipid component is selected from a combination of 18-(oleoyloxy) octadecanoic acid, or a pharmaceutically acceptable salt thereof, and behenyl oleate in about a 1:1 molar ratio and a combination of 20-(oleoyloxy) eicosanoic acid, or a pharmaceutically acceptable salt thereof, and behenyl oleate in a molar ratio of about 1:1. Yet more particularly, the lipid component is 18-(oleoyloxy) octadecanoic acid, or a pharmaceutically acceptable salt thereof, and behenyl oleate in molar ratio of about 1:1. Such lipid components may also contain the fatty acid ester of a hydroxy fatty acid and wax ester in the corresponding mass ratios)

More particularly, the lipid component is selected from a combination of 18-(oleoyloxy) octadecanoic acid and behenyl oleate in about a 1:1 molar ratio and a combination of 20-(oleoyloxy) eicosanoic acid and behenyl oleate in a molar ratio of about 1:1. Yet more particularly, the lipid component is 18-(oleoyloxy) octadecanoic acid and behenyl oleate in molar ratio of about 1:1. Such lipid components may also contain the fatty acid ester of a hydroxy fatty acid and wax ester in the corresponding mass ratios.

Further combinations of a fatty acid ester of a hydroxy fatty acid, or a pharmaceutically acceptable salt thereof and wax ester that may be used as the lipid component in the composition of the invention include combinations of a fatty acid ester of a hydroxy fatty acid selected from
18-(oleoyloxy) octadecanoic acid,
20-(oleoyloxy) eicosanoic acid,
18-(oleyloxy)-18-oxo-octadecanoic acid,
22-(palmitoleoyloxy) behenic acid;
22-(palmitoleyloxy)-22-oxo-behenic acid;
20-(palmitoleyloxy)-20-oxo-eicosanoic acid; and
18-(palmitoleyloxy)-18-oxo-octadecanoic acid,
or a pharmaceutically acceptable salt thereof;
with a wax ester selected from
behenyl oleate;
behenyl linoleate;
behenyl behenate;
behenyl stearate; and
arachidyl laurate,
wherein, for each combination, the fatty acid ester of a hydroxy fatty acid and wax ester are present in a molar ratio of about 1:1 to about 1:9 (fatty acid ester of a hydroxy fatty acid:wax ester), for example from about 1:1 to about 1:5, such as from about 1:1 to about 1:3 (e.g. from about 1:1 to about 1:2). More particularly, the fatty acid ester of a hydroxy fatty acid and wax ester are present in a molar ratio of about 1:1.

Further combinations of a fatty acid ester of a hydroxy fatty acid, or a pharmaceutically acceptable salt thereof and wax ester that may be used as the lipid component in the composition of the invention include combinations of a fatty acid ester of a hydroxy fatty acid selected from
18-(oleoyloxy) octadecanoic acid,
20-(oleoyloxy) eicosanoic acid,
18-(oleyloxy)-18-oxo-octadecanoic acid,
22-(palmitoleoyloxy) behenic acid;
22-(palmitoleyloxy)-22-oxo-behenic acid;
20-(palmitoleyloxy)-20-oxo-eicosanoic acid; and
18-(palmitoleyloxy)-18-oxo-octadecanoic acid,
or a pharmaceutically acceptable salt thereof;
with a wax ester selected from
behenyl oleate;
behenyl stearate; and
arachidyl laurate,
wherein, for each combination, the fatty acid ester of a hydroxy fatty acid and wax ester are present in a molar ratio of about 1:1 to about 1:9 (fatty acid ester of a hydroxy fatty acid:wax ester), for example from about 1:1 to about 1:5, such as from about 1:1 to about 1:3 (e.g. from about 1:1 to about 1:2). More particularly, the fatty acid ester of a hydroxy fatty acid and wax ester are present in a molar ratio of about 1:1.

Further combinations of a fatty acid ester of a hydroxy fatty acid, or a pharmaceutically acceptable salt thereof and wax ester that may be used as the lipid component in the composition of the invention include combinations of a fatty acid ester of a hydroxy fatty acid selected from
18-(oleyloxy)-18-oxo-octadecanoic acid,
22-(palmitoleoyloxy) behenic acid;
22-(palmitoleyloxy)-22-oxo-behenic acid;
20-(palmitoleyloxy)-20-oxo-eicosanoic acid; and
18-(palmitoleyloxy)-18-oxo-octadecanoic acid,
or a pharmaceutically acceptable salt thereof;
with a wax ester selected from
behenyl oleate;
behenyl stearate; and
arachidyl laurate,
wherein, for each combination, the fatty acid ester of a hydroxy fatty acid and wax ester are present in a molar ratio of about 1:1 to about 1:9 (fatty acid ester of a hydroxy fatty acid:wax ester), for example from about 1:1 to about 1:5, such as from about 1:1 to about 1:3 (e.g. from about 1:1 to about 1:2). More particularly, the fatty acid ester of a hydroxy fatty acid and wax ester are present in a molar ratio of about 1:1.

In further particular embodiments, the lipid component is a fatty alcohol of formula III or formula IV, or a pharmaceutically acceptable salt thereof, preferably wherein the lipid component is selected from

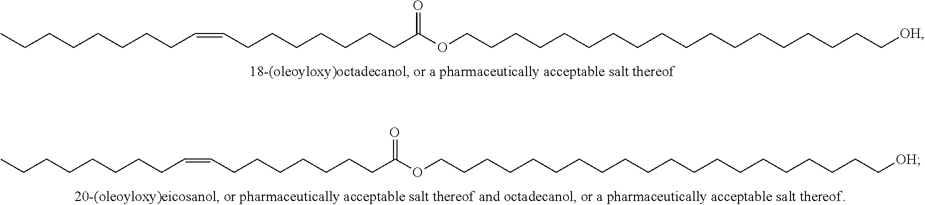 [SMILES: CCCCCCCC/C=C\CCCCCCCC(=O)OCCCCCCCCCCCCCCCCCCO ; CCCCCCCC/C=C\CCCCCCCC(=O)OCCCCCCCCCCCCCCCCCCCCO]

More particularly, the fatty alcohol is octadecanol, or a pharmaceutically acceptable salt thereof.

In further particular embodiments, the lipid component is a fatty alcohol of formula III or formula IV, or a pharmaceutically acceptable salt thereof, preferably wherein the lipid component is selected from

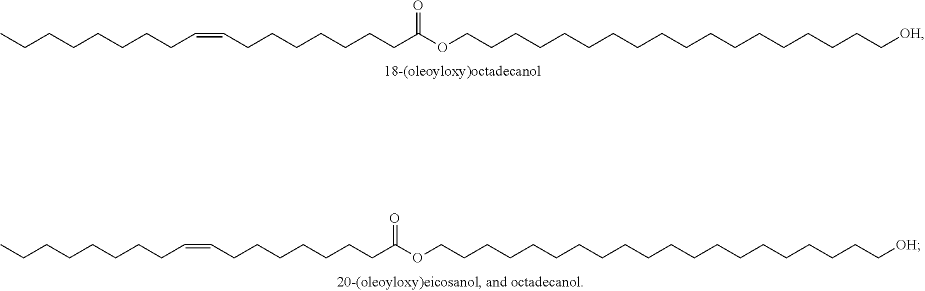 [SMILES: CCCCCCCC/C=C\CCCCCCCC(=O)OCCCCCCCCCCCCCCCCCCO ; CCCCCCCC/C=C\CCCCCCCC(=O)OCCCCCCCCCCCCCCCCCCCCO]

More particularly, the fatty alcohol is octadecanol.

In further particular embodiments, the lipid component is a combination of a fatty acid ester of a hydroxy fatty acid of formula I, or pharmaceutically-acceptable salt thereof with a fatty alcohol of formula III or IV, or a pharmaceutically acceptable salt thereof, wherein the components are present in mass ratio of from about 3:1 to about 1:3, such as a mass ratio of from about 2:1 to about 1:2, such as about 1:1. In more particular embodiments, the fatty acid ester of a hydroxy fatty acid is 18-(oleoyloxy) octadecanoic acid (optionally in the form of a pharmaceutically acceptable salt) and the fatty alcohol is 1-octadecanol (optionally in the form of a pharmaceutically acceptable salt).

In further particular embodiments, the lipid component is a combination of a wax ester of formula II with a fatty alcohol of formula III or IV, or a pharmaceutically acceptable salt thereof, wherein the components are present in a mass ratio from about 3:1 to about 1:3, such as a mass ratio of from about 2:1 to about 1:2, such as about 1:1. In more particular embodiments, the wax ester is behenyl oleate and the fatty alcohol is 1-octadecanol (optionally in the form of a pharmaceutically acceptable salt).

The phospholipid component of the liposomes and compositions described herein is selected from the group consisting of a phosphatidylcholine, a phosphatidylglycerol, or a pharmaceutically acceptable salt thereof, a phosphatidylserine, or a pharmaceutically acceptable salt thereof, a phosphatidic acid, or a pharmaceutically acceptable salt thereof, a phosphatidylethanolamine, and a mixture thereof. In particular, the phospholipid component may be a single phospholipid selected from the group consisting of a phosphatidylcholine, a phosphatidylglycerol, or a pharmaceutically acceptable salt thereof, a phosphatidylserine, or a pharmaceutically acceptable salt thereof, a phosphatidic acid, or a pharmaceutically acceptable salt thereof, and a phosphatidylethanolamine.

In particular embodiments, the phospholipid component is selected from phosphatidylcholines, phosphatidylglycerols, phosphatidylserines, phosphatidic acids, and phosphatidylethanolamines with a transition (melting) temperature (Tm) of from about 50° C. to about 70° C. (at atmospheric pressure). Such phospholipids have been found to produce highly evaporation resistant formulations in combination with the lipid components described herein.

The skilled person will understand that the transition temperature of a phospholipid is the temperature at which the lipid melts from the gel to the liquid phase.

Examples of phospholipids with a transition (melting) temperature in the range of about 50° C. to about 70° C. that may be used (alone or in combination (e.g. alone)) as the phospholipid component of the compositions and liposomes described herein include the following:
1,2-distearoyl-sn-glycero-3-phosphocholine, (DSPC),
1,2-diarachidoyl-sn-glycero-3-phosphatidylcholine (DAPC),
17:0-phosphatidyl choline,
19:0-phosphatidyl choline,
20:0-phosphatidyl choline,
1,2-distearoyl-sn-glycero-3-phosphoglycerol (DSPG), or a pharmaceutically acceptable salt thereof (e.g. as a sodium salt),
1,2-dipalmitoyl-sn-glycero-3-phosphoserine (DPPS), or a pharmaceutically acceptable salt thereof (e.g. as a sodium salt),
1,2-distearoyl-sn-glycero-3-phospho-L-serine (DSPS), or a pharmaceutically acceptable salt thereof (e.g. as a sodium salt),
1,2-dimyristoyl-sn-glycero-3-phosphate (DMPA), or a pharmaceutically acceptable salt thereof (e.g. as a sodium salt)
1,2-dipalmitoyl-sn-glycero-3-phosphate (DPPA), or a pharmaceutically acceptable salt thereof (e.g. as a sodium salt),
1,2-dimyristoyl-sn-glycero-3-phosphoethanolamine (DMPE), and
1,2-dipalmitoyl-sn-glycero-3-phosphoethanolamine (DPPE).

In further embodiments, phospholipid component is selected from phosphatidylcholines, phosphatidylglycerols, phosphatidylserine, phosphatidic acids, and phosphatidylethanolamines with a transition (melting) temperature (Tm) of from about 24° C. to about 35° C. (at atmospheric pressure). Such phospholipids have a transition temperature between room temperature and body temperature and therefore their phase behaviour can change after application to a body surface (e.g. to the eye). Phospholipids with transition temperatures in this range may be referred to as ‘thermoresponsive phospholipids’.

Examples of phospholipids with a transition (melting) temperature in the range of about 24° C. to about 35° C. that may be used (alone or in combination (e.g. alone)) as the phospholipid component of the compositions and liposomes described herein include the following:
1,2-dimyristoyl-sn-glycero-3-phosphocholine
1,2-dipentadecanoyl-sn-glycero-3-phosphocholine (C15:0 phosphatidyl choline),
14:0-16:0-phosphatidyl choline,
18:0-14:0-phosphatidyl choline,
1,2-dimyristoyl-sn-glycero-3-phosphoglycerol (DMPG), or a pharmaceutically acceptable salt thereof, (e.g. as a sodium salt),
1,2-dimyristoyl-sn-glycero-3-phospho-L-serine (DMPS), or a pharmaceutically acceptable salt thereof, (e.g. as a sodium salt),
1,2-dilauroyl-sn-glycero-3-phosphate (DLPA), or a pharmaceutically acceptable salt thereof, (e.g. as a sodium salt), and
1,2-dilauroyl-sn-glycero-3-phosphoethanolamine (DLPE).

The nomenclature X:Y when used in reference to the lipid and phospholipids described herein may be considered to take its normal meaning in the field of lipid research. In particular, X may be understood to indicate the number of carbon atoms present in the fatty acid species (or fatty acid derivative (such as a fatty alcohol or fatty acid ester) present in the lipid or phospholipid species and Y may be understood to indicate the number of carbon-carbon double bonds present in the species. For example, 18:0 may be understood to refer to a species (e.g. a fatty acid or fatty acid component of a lipid or phospholipid) containing 18 carbon atoms and 0 double bonds.

Further phospholipids with properties suitable for inclusion in the compositions of the invention include:
1,2-dipalmitoyl-sn-glycero-3-phosphoethanolamine (16:0 PE),
1,2-distearoyl-sn-glycero-3-phosphoethanolamine (18:0 PE),
1,2-distearoyl-sn-glycero-3-phospho-rac-(1-glycerol), or a pharmaceutically acceptable salt thereof (e.g. a sodium salt) (18:0 PG), and
1,2-distearoyl-sn-glycero-3-phospho-L-serine, or a pharmaceutically acceptable salt thereof (e.g. a sodium salt) (18:0 PS).

More particularly, phospholipids with properties suitable for inclusion in the compositions of the invention include:
1,2-distearoyl-sn-glycero-3-phosphoethanolamine (18:0 PE),
1,2-distearoyl-sn-glycero-3-phospho-rac-(1-glycerol), or a pharmaceutically acceptable salt thereof (e.g. a sodium salt) (18:0 PG),

In further particular embodiments, the phospholipid component comprises (consists essentially of, or consists of a phospholipid (a phosphatidylcholine, phosphatidylglycerol, a phosphatidylserine, a phosphatidic acid, or a phosphatidylethanolamine) having a chain length in the C₁₄-C₂₂ range (more particularly the C₁₄ to C₂₀ range)

Phosphatidyl cholines have been found to display particularly favourable properties in the compositions of the invention, in terms of their anti-evaporative properties and phase behaviour. Thus, in further particular embodiments, the phospholipid component is a C₁₄-C₂₀ phosphatidylcholine, or a mixture thereof. The skilled person will understand that phosphatidylcholines are a class of phospholipids incorporating choline as a head group. As used herein, the phrase C₁₄-C₂₀ phosphatidylcholine(s) may be understood to refer to phosphatidylcholines wherein the two fatty acid groups contain between 14 and 20 carbons, which may be saturated or unsaturated, and may be the same or different.

In particular embodiments, the phospholipid component is selected from the group consisting of 1,2-dimyristoyl-sn-glycero-3-phosphocholine (1,2-Dimyristoyl-sn-glycero-3-phosphatidylcholine) (DMPC), 1,2-dipentadecanoyl-sn-glycero-3-phosphocholine (C15:0 phosphatidyl choline), dipalmitoylphosphatidylcholine (DPPC), 1,2-distearoyl-sn-glycero-3-phosphocholine (1,2-distearoyl-sn-glycero-3-phosphatidylcholine) (DSPC), 1,2-diarachidoyl-sn-glycero-3-phosphatidylcholine (1,2-diarachidoyl-sn-glycero-3-phosphocholine) (DAPC), 1,2-dioleoyl-sn-glycero-3-phosphocholine (1,2-dioleoyl-sn-glycero-3-phosphatidylcholine) (DOPC), and mixtures thereof.

More particularly, the phospholipid component is selected from the group consisting of 1,2-distearoyl-sn-glycero-3-phosphocholine (DSPC), 1,2-diarachidoyl-sn-glycero-3-phosphatidylcholine (DAPC), 1,2-dioleoyl-sn-glycero-3-phosphocholine (DOPC), and mixtures thereof. More particularly, the phospholipid component is selected from the group consisting of 1,2-distearoyl-sn-glycero-3-phosphocholine (DSPC), 1,2-diarachidoyl-sn-glycero-3-phosphatidylcholine (DAPC), and mixtures thereof. Yet more particularly, the phospholipid component is selected from the group consisting of 1,2-distearoyl-sn-glycero-3-phosphocholine (DSPC) and 1,2-diarachidoyl-sn-glycero-3-phosphatidylcholine (DAPC).

In yet further embodiments, the phospholipid component is DMPC. Such embodiments have been found to allow high lipid loadings in the compositions of the invention.

In yet further embodiments, the phospholipid component is 1,2-distearoyl-sn-glycero-3-phosphocholine (DSPC).

In yet further embodiments, the phospholipid component is 1,2-diarachidoyl-sn-glycero-3-phosphatidylcholine (DAPC).

Particular compositions of the invention that may be mentioned include those in which the lipid component is selected from:
(i) a fatty acid ester of a hydroxy fatty acid of formula I, or a pharmaceutically acceptable salt thereof, as defined in any one of the preceding claims; and
(ii) a combination of a fatty acid ester of a hydroxy fatty acid of formula I, or a pharmaceutically acceptable salt thereof, and a wax ester of formula II, as defined in any of the preceding claims, wherein the fatty acid ester of a hydroxy fatty acid of formula I and the wax ester of formula II are present in a molar ratio of from about 1:1 to about 1:9, such as about 1:1; and
the phospholipid component is a phosphatidyl choline selected from the group consisting of 1,2-distearoyl-sn-glycero-3-phosphocholine (DSPC), 1,2-diarachidoyl-sn-glycero-3-phosphatidylcholine (DAPC) (particularly DAPC).

In particular embodiments of such compositions, the lipid component is selected from selected from the group consisting of:

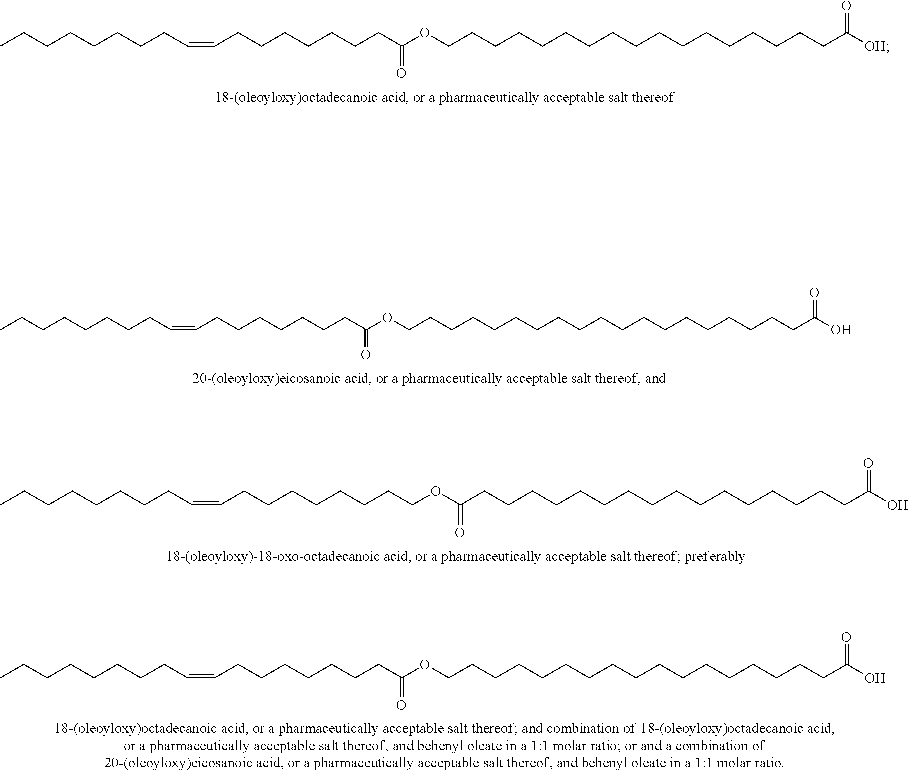 [SMILES: CCCCCCCC/C=C\CCCCCCCC(=O)OCCCCCCCCCCCCCCCCCC(=O)O ; CCCCCCCC/C=C\CCCCCCCC(=O)OCCCCCCCCCCCCCCCCCCCC(=O)O ; CCCCCCCC/C=C\CCCCCCCCOC(=O)CCCCCCCCCCCCCCCCC(=O)O ; CCCCCCCC/C=C\CCCCCCCC(=O)OCCCCCCCCCCCCCCCCCC(=O)O]

The compositions of the invention comprise liposomes comprising a lipid component and a phospholipid component as defined herein. In particular, it may be understood that the lipid and phospholipid components are entirely incorporated into the liposomes and not otherwise free in the compositions. As used herein, references to the lipid component (or individual lipid species) being incorporated into the liposomes may be understood to indicate that the lipid component (which may, where appropriate, contain more than one lipid species) is integrated into the lipid bilayer formed by the phospholipids.

In particular embodiments of the compositions of the invention, at least about 70% (i.e. about 80% to 100%) (by mass) of the lipid and phospholipid components present in the compositions (by mass) are incorporated into the liposomes. More particularly, at least about 80% (i.e, about 80% to 100%) (by mass) of the lipid and phospholipid components present in the compositions (by mass) are incorporated into the liposomes. Yet more particularly, at least about 90% (i.e, about 90% to 100%) (by mass) of the lipid and phospholipid components present in the compositions are incorporated into the liposomes. Yet more particularly, at least about 95% (by mass), such as at least about 98% (by mass) (e.g. at least about 99% (by mass)) of the lipid and phospholipid components present in the compositions are incorporated into the liposomes.

In particular embodiments, the compositions of the invention do not contain any other lipid species (other than the lipid and phospholipid components defined herein).

In further particular embodiments, the liposomes contained in the compositions of the invention do not comprise any additional lipid species (other than the lipid and phospholipid components defined herein).

In further particular embodiments, the liposomes contained in the compositions of the invention consist essentially of the lipid and phospholipid components as defined herein. In yet further particular embodiments, the liposomes contained within the compositions of the invention consist of the lipid and phospholipid components as defined herein.

The skilled person will understand that liposomes and liposomal formulations are typically administered as aqueous solutions. Therefore, in particular embodiments, the compositions of the invention also comprise water, such compositions may be referred to as ‘aqueous compositions’. Such compositions will also generally be liquid (at room temperature and pressure (for example 25° C. and 1 atmosphere).

In the compositions defined herein, the lipid and phospholipid components are preferably present in a ratio of from about 2:1 (lipid:phospholipid) to about 1:10 by mass, such as from about 1:1 to about 1:8 by mass, for example in a ratio of from about 1:1 to 1:4 by mass, such as in a ratio of about 1:1-1:3 by mass (e.g. in a ratio of from about 1:1-1:2 by mass). In certain embodiments, the ratio of the lipid and phospholipid components is from about 1:2 to about 1:3 by mass, such as about 1:2 by mass.

The liposomes in the compositions of the invention preferably have a size (diameter) in the nanoparticle range (which may be understood to be below about 500 nm in diameter). In particular, the liposomes have a diameter of from about 100 to about 400 nm, such as from about 100 to about 300 nm, for example from about 100 to about 200 nm. Preferably at least 90% (such as at least 95% (e.g, at least 99%) of the nanoparticles are smaller than 500 nm in diameter, more particularly at least 90% (such as at least 95% (e.g. at least 99%) of the nanoparticles are smaller than 400 nm in diameter. Yet more particularly, at least 90% (such as at least 95% (e.g. at least 99%) of the nanoparticles are smaller than 300 nm in diameter.

The skilled person will understand that the liposomes of the invention will usually be administered in the form of a composition or formulation (in particular, in the form of pharmaceutical compositions or formulations-denoting a composition or formulation suitable for pharmaceutical use).

Accordingly, in a further aspect of the invention, there is provided a pharmaceutical composition comprising a liposome of the invention as described herein.

In particular embodiments, the combined amount of the lipid component and phospholipid component (i.e. lipid component+phospholipid component) in the compositions of the invention is from about 0.1% (w/v) to about 20% (w/v). In particular embodiments, the combined amount of the lipid component and phospholipid component is the total lipid content of the compositions. In other embodiments, further lipid species may be included, as appropriate.

More particularly, the combined amount of the lipid and phospholipid components is from about 0.5% (w/v) to about 18% (w/v), such as from about 1% (w/v) to about 17% (w/v), for example from about 1% (w/v) to about 15% (w/w).

In further particular embodiments, such as those in which the phospholipid components is selected from the group consisting of DAPC, DSPC and mixtures thereof (e.g DAPC or DSPC alone) the total amount of the lipid and phospholipid components is from about 0.5% (w/v) to about 8% (w/v), such as from about 0.5% (w/v) to about 6% (w/v), such as from about 0.5% (w/v) to about 3.5% (w/v).

In further particular embodiments, such as those in which the phospholipid component is DMPC, the combined amount of the lipid and phospholipid components is in the range of from about 1% (w/v) to about 17% (w/v), for example from about 1% (w/v) to about 15% (w/v), such as from about 1% (w/v) to about 12% (w/v), e.g. from about 1% (w/v) to about 10% (w/v).

In further particular embodiments, the lipid component of the compositions, as defined herein, is present in an amount of from about 0.1 (w/v) to about 10% (w/v), such as from about 0.5% (w/v) to about 8% (w/v) for example from about 0.5% (w/v) to about 5% (w/v) (e.g. from about 0.5% to about 3% (w/v)).

In further particular embodiments, the phospholipid component of the compositions, as defined herein, is present in an amount of from about 0.1% (w/v) to about 10% (w/v), such as from about 0.5% (w/v) to about 8% (w/v) for example from about 0.5% (w/v) to about 5% (w/v) (e.g. from about 0.5% to about 2% (w/v)).

Particular compositions of the invention that may be mentioned include those in which:
The lipid component is a fatty acid ester of a hydroxy fatty acid selected from 18-(oleoyloxy) octadecanoic acid and 18-(oleyloxy)-18-oxo-octadecanoic acid and is present in an amount of from about 0.5% (w/v) to about 2% (w/v), and the phospholipid component is selected from DAPC and DSPC and is present in an amount of from about 0.5% (w/v) to about 5% (w/v) (e.g. 0.5% (w/v) to about 2.5% (w/v); The lipid component is a 1:1 mixture (mass ratio, or molar ratio (e.g molar ratio) of a fatty acid ester of a hydroxy fatty acid selected from 18-(oleoyloxy) octadecanoic acid and 18-(oleyloxy)-18-oxo-octadecanoic acid and behenyl oleate, present in a combined amount of from about 5% (w/v) to about 2% (w/v), and the phospholipid component is selected from DAPC and DSPC and is present in an amount of from about 0.5% (w/v) to about 5% (w/v) (e.g. 0.5% (w/v) to about 2.5% (w/v);
The lipid component is 1-octadecanol present in an amount of from about 0.5% (w/v) to about 2% (w/v), and the phospholipid component is selected from DAPC and DSPC and is present in an amount of from about 0.5% (w/v) to about 5% (w/v) (e.g. 0.5% (w/v) to about 2.5% (w/v);
The lipid component is a 1:1 mixture by weight of octadecanol and behenyl oleate, present in a combined amount of from about 5% (w/v) to about 2% (w/v), and the phospholipid component is selected from DAPC and DSPC and is present in an amount of from about 0.5% (w/v) to about 5% (w/v) (e.g. 0.5% (w/v) to about 2.5% (w/v) The lipid component is a 1:1 mixture by weight of a fatty acid ester of a hydroxy fatty acid selected from 18-(oleoyloxy) octadecanoic acid and 18-(oleyloxy)-18-oxo-octadecanoic acid and 1-octadecanol, present in a combined amount of from about 5% (w/v) to about 2% (w/v), and the phospholipid component is selected from DAPC and DSPC and is present in an amount of from about 0.5% (w/v) to about 5% (w/v) (e.g. 0.5% (w/v) to about 2.5% (w/v)).

The compositions of the invention may further comprise additional components or excipients, such as an additional lipid species, pH adjusting or buffering agents, tonicity adjusting agents, stabilisers, thickening agents, preservatives, and the like.

The compositions of the invention are intended to be applied topically to the eye surface (e.g. in the form of an aqueous liquid composition). Accordingly, the compositions may further contain pharmaceutically acceptable excipients suitable for topical administration to the eye, such as those listed in the European Pharmacopeia (in particular the 11th Edition). In particular, the compositions of the invention should be isotonic and have pH of around neutral (for example a pH of from about 5 to about 9, or about 6 to about 8, such as from about 6.5 to about 7.5 (e.g, about pH 7)).

The compositions of the invention may particularly be in the form of liposomal suspensions (wherein the lipid and phospholipid components as defined herein are incorporated into the liposomes). The compositions of the invention may particularly be in the form of a spray or eye drops.

Appropriate dosages and dosage forms of the compositions of the invention may be determined by the skilled person, taking into consideration factors such as type and severity of the condition that is to be treated, as well as the species, age, weight, sex, renal function, hepatic function and response of the particular patient to be treated.

In particular, it is envisaged that the compositions of the invention may be administered in the form of eye drops with one to two drops of the composition administered once or twice per day. Particularly when the patient is experiencing symptoms to be alleviated.

Thus, in particular embodiments, the compositions of the invention are provided in the form of eye drops, optionally as a kit-of-parts comprising the composition and a suitable applicator for topical administration to the eye.

### Liposomes of the Invention

As discussed herein, the compositions comprise liposomes comprising a lipid component and a phospholipid component as defined herein.

Accordingly, in a further aspect of the invention, there is provide a liposome comprising a lipid component and a phospholipid component as defined herein, wherein the lipid and phospholipid components are present in a ratio of from about 10:1 to about 1:10 by mass.

In a further aspect of the invention, there is provided a pharmaceutical composition comprising a liposome comprising a lipid component and a phospholipid component as defined herein, wherein the lipid and phospholipid components are present in a ratio of from about 10:1 to about 1:10 by mass. Such compositions may preferably further comprise water and be liquid at room temperature and pressure. All other preferred and particular aspects of the compositions of the invention described herein also apply to this aspect.

In such liposomes and compositions comprising such liposomes, the lipid and phospholipid components are preferably present in a ratio of from about 2:1 (lipid:phospholipid) to about 1:10 by mass, such as from about 1:1 to about 1:8 by mass, for example in a ratio of from about 1:1 to 1:4 by mass, such as in a ratio of about 1:1-1:3 by mass (e.g. in a ratio of from about 1:1-1:2 by mass). In certain embodiments, the ratio of the lipid:phospholipid components is from about 1:2 to about 1:3 by mass, such as about 1:2 by mass.

### Compounds of the Invention

Certain fatty acid esters of hydroxy fatty acids are new and have improved properties in terms of evaporation resistance compared to similar compounds reported in the prior art. Therefore, in a further aspect of the invention, there is provided a compound selected from the group consisting of:
18-(oleoyloxy) octadecanoic acid,
15-(oleyloxy)-15-oxo-pentadecanoic acid
18-(oleyloxy)-18-oxo-octadecanoic acid
20-(oleyloxy)-20-oxo-eicosanoic acid,
20-(palmitoleoyloxy) eicosanoic acid; and
18-(palmitoleoyloxy) octadecanoic acid;
or a pharmaceutically acceptable salt thereof.

There is further provided a pharmaceutical composition comprising a compound selected from the group consisting of
18-(oleoyloxy) octadecanoic acid,
15-(oleyloxy)-15-oxo-pentadecanoic acid
18-(oleyloxy)-18-oxo-octadecanoic acid
20-(oleyloxy)-20-oxo-eicosanoic acid,
20-(palmitoleoyloxy) eicosanoic acid; and
18-(palmitoleoyloxy) octadecanoic acid
or a pharmaceutically acceptable salt thereof.

More particularly, there is provided a compound selected from the group consisting of:
15-(oleyloxy)-15-oxo-pentadecanoic acid
18-(oleyloxy)-18-oxo-octadecanoic acid
20-(oleyloxy)-20-oxo-eicosanoic acid,
20-(palmitoleoyloxy) eicosanoic acid; and
18-(palmitoleoyloxy) octadecanoic acid;
or a pharmaceutically acceptable salt thereof.

There is further provided a pharmaceutical composition comprising a compound selected from the group consisting of:
15-(oleyloxy)-15-oxo-pentadecanoic acid
18-(oleyloxy)-18-oxo-octadecanoic acid
20-(oleyloxy)-20-oxo-eicosanoic acid,
20-(palmitoleoyloxy) eicosanoic acid; and
18-(palmitoleoyloxy) octadecanoic acid;
or a pharmaceutically acceptable salt thereof.

There is further provided a compound selected from the group consisting of
20-(palmitoleoyloxy) eicosanoic acid;
18-(palmitoleoyloxy) octadecanoic acid; and
18-(oleoyloxy) octadecanoic acid.

There is further provided a pharmaceutical composition comprising a compound selected from the group consisting of:
20-(palmitoleoyloxy) eicosanoic acid;
18-(palmitoleoyloxy) octadecanoic acid; and
18-(oleoyloxy) octadecanoic acid.

There is further provided a compound selected from the group consisting of:
15-(oleyloxy)-15-oxo-pentadecanoic acid
18-(oleyloxy)-18-oxo-octadecanoic acid
20-(oleyloxy)-20-oxo-eicosanoic acid,
or a pharmaceutically acceptable salt thereof.

There is further provided a pharmaceutical composition comprising a compound selected from the group consisting of:
15-(oleyloxy)-15-oxo-pentadecanoic acid
18-(oleyloxy)-18-oxo-octadecanoic acid
20-(oleyloxy)-20-oxo-eicosanoic acid,
or a pharmaceutically acceptable salt thereof.

There is further provided a compound selected from the group consisting of:
18-(oleyloxy)-18-oxo-octadecanoic acid
20-(oleyloxy)-20-oxo-eicosanoic acid,
or a pharmaceutically acceptable salt thereof.

There is further provided a pharmaceutical composition comprising a compound selected from the group consisting of:
18-(oleyloxy)-18-oxo-octadecanoic acid
20-(oleyloxy)-20-oxo-eicosanoic acid,
or a pharmaceutically acceptable salt thereof.

There is further provided a compound selected from the group consisting of
22-(palmitoleoyloxy) behenic acid
22-(palmitoleyloxy)-22-oxo-behenic acid
20-(palmitoleyloxy)-20-oxo-eicosanoic acid; and
18-(palmitoleyloxy)-18-oxo-octadecanoic acid,
or a pharmaceutically acceptable salt thereof.

There is further provided a pharmaceutical composition comprising a compound selected from the group consisting of
22-(palmitoleoyloxy) behenic acid
22-(palmitoleyloxy)-22-oxo-behenic acid
20-(palmitoleyloxy)-20-oxo-eicosanoic acid; and
18-(palmitoleyloxy)-18-oxo-octadecanoic acid,
or a pharmaceutically acceptable salt thereof.

In particular, there is provided a compound selected from the group consisting of
15-(oleyloxy)-15-oxo-pentadecanoic acid
18-(oleyloxy)-18-oxo-octadecanoic acid;
20-(oleyloxy)-20-oxo-eicosanoic acid;
22-(palmitoleyloxy)-22-oxo-behenic acid
20-(palmitoleyloxy)-20-oxo-eicosanoic acid; and
18-(palmitoleyloxy)-18-oxo-octadecanoic acid,
or a pharmaceutically acceptable salt thereof.

There is further provided a pharmaceutical composition comprising a compound selected from the group consisting of:
15-(oleyloxy)-15-oxo-pentadecanoic acid
18-(oleyloxy)-18-oxo-octadecanoic acid;
20-(oleyloxy)-20-oxo-eicosanoic acid;
22-(palmitoleyloxy)-22-oxo-behenic acid
20-(palmitoleyloxy)-20-oxo-eicosanoic acid,
18-(palmitoleyloxy)-18-oxo-octadecanoic acid, and
or a pharmaceutically acceptable salt thereof.

In particular, there is provided the compound 18-(oleoyloxy) octadecanoic acid, or a pharmaceutically acceptable salt thereof, and a pharmaceutical composition comprising the same.

Such compositions (compositions comprising the compounds of the invention) may contain pharmaceutically acceptable excipients, such as those described herein (including particularly phospholipids as described herein). In particular, pharmaceutically acceptable excipients may include vehicles, adjuvants, carriers, diluents, pH adjusting and buffering agents, tonicity adjusting agents, stabilizers, wetting agents and the like. In particular, such excipients may include adjuvants, diluents or carriers.

Such formulations may particularly be formulated for topical use for application to the eye surface, as for the other compositions described herein.

In particular embodiments, the pharmaceutical composition comprising 18-(oleoyloxy) octadecanoic acid, or a pharmaceutically acceptable salt thereof, does not comprise a wax ester selected from the group consisting of behenyl behenoate (BB), arachidyl laurate, behenyl oleate.

In further particular embodiments, the pharmaceutical composition comprising 18-(oleoyloxy) octadecanoic acid, or a pharmaceutically acceptable salt thereof, does not comprise a wax ester (i.e. esters of a fatty acid and a fatty alcohol), or a structural analogue thereof.

In further particular embodiments, the pharmaceutical composition comprising 18-(oleoyloxy) octadecanoic acid, or a pharmaceutically acceptable salt thereof, does not comprise:
(i) a wax ester, or structural analogue thereof, with no maximum chain length;
(ii) a wax ester, or structural analogue thereof, with a chain length of C15-C100, C19-C72, C20-C55, C20-C50, C20-C40, C20-C35, C20-C25, C25-C45, C25-C40, C25-C35 or C25-C30;
(ii) a wax ester, or analogue thereof, with a chain length of C20, C21, C22, C23, C24, C25, C26, C27, C28, C29, C30, C31, C32, C33, C34, C35, C36, C37, C38, C39, C40, C41, C42, C43, C44, C45, C46, C47, C48, C49, C50, C51, C52, C53, C54 or C55; and/or
(iv) a linear or branched wax ester.

In further particular embodiments, the pharmaceutical composition comprising 18-(oleoyloxy) octadecanoic acid, or a pharmaceutically acceptable salt thereof, does not contain a wax ester, or structural analogue thereof, of formula (ii):

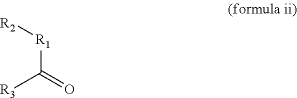

wherein
R₁ is a carbon atom, an oxygen atom or a nitrogen atom;
R₂ is a linear or branched C9-C50 alkyl, alkenyl or alkynyl chain, or a structural analogue thereof;
R₃ is a linear or branched C9-C50 alkyl, alkenyl or alkynyl chain, or a structural analogue thereof.

In particular, the composition comprising 18-(oleoyloxy) octadecanoic acid, or a pharmaceutically acceptable salt thereof, does not comprise a wax ester as described and/or listed in patent application no. PCT/EP2022/061585.

### Medical Uses

The compositions, liposomes, and compounds of the invention are useful as pharmaceuticals. Therefore, in a further embodiment, there is provided the liposomes, compositions and compounds of the invention, as described herein, for use in medicine.

In particular, the compositions, liposomes, and compounds of the invention are useful in the treatment and/or prevention of ocular surface disorders, such as dry eye disease and meibomian gland dysfunction. The liposomes, compositions and compounds of the invention are also of potential utility in the treatment and/or of allergic conjunctivitis.

Accordingly, in a further aspect of the invention, there is provided the compositions, liposomes and compounds of the invention, as defined herein, for use in the treatment and/or prevention of an ocular surface disorder or allergic conjunctivitis.

In an alternative aspect of the invention, there is provided a method for treating and/or preventing an ocular surface disorder or allergic conjunctivitis, comprising administering to a patient in need thereof a therapeutically effective amount of a composition, liposome or compound of the invention.

In a further alternative aspect of the invention, there is provided the use of the compositions, liposomes and compounds of the invention, as defined herein, in the manufacture of a medicament for the treatment and/or prevention of an ocular surface disorder or allergic conjunctivitis.

The skilled person will understand that references to the treatment of a particular condition (or, similarly, to treating that condition) will take their normal meanings in the field of medicine. In particular, the terms may refer to achieving a reduction in the severity and/or frequency of occurrence of one or more clinical symptom associated with the condition, as adjudged by a physician attending a patient having or being susceptible to such symptoms. For example, in the case of dry eye disease, the term may refer to achieving a reduction in any stinging or burning sensation, blurred vision and/or redness, or the reduction in any other clinically relevant symptom.

As used herein, the term prevention (and, similarly, preventing) will include references to the prophylaxis of the disease or disorder (and vice-versa). As such, references to prevention may also be references to prophylaxis, and vice versa. In particular, such terms term may refer to achieving a reduction (for example, at least a 10% reduction, such as at least a 20%, 30% or 40% reduction, e.g. at least a 50% reduction) in the likelihood of the patient (or healthy subject) developing the condition (which may be understood as meaning that the condition of the patient changes such that patient is diagnosed by a physician as having, e.g. requiring treatment for, the relevant disease or disorder).

As used herein, references to a patient (or to patients) will refer to a living subject being treated, including mammalian (e.g. human) patients. In particular, references to a patient will refer to human patients.

For the avoidance of doubt, the skilled person will understand that such treatment or prevention will be performed in a patient (or subject) in need thereof. The need of a patient (or subject) for such treatment or prevention may be assessed by those skilled in the art using routine techniques.

As used herein, the terms disease and disorder (and, similarly, the terms condition, illness, medical problem, and the like) may be used interchangeably.

As used herein, the term effective amount will refer to an amount of a compound, composition or liposome that confers a therapeutic effect on the treated patient. The effect may be observed in a manner that is objective (i.e. measurable by some test or marker) or subjective (i.e, the subject gives an indication of and/or feels an effect). In particular, the effect may be observed (e.g. measured) in a manner that is objective, using appropriate tests as known to those skilled in the art. For example, in the case of dry eye disease, the compositions, compounds or liposome of the invention may achieve an improvement in tear film breakup time compared to before treatment began.

In particular embodiments of the compositions, liposomes and compounds for use, methods and uses described herein, the ocular surface disorder is selected from the group consisting of dry eye disease, meibomian gland dysfunction and blepharitis.

In particular embodiments of the compositions, liposomes and compounds for use, methods and uses described herein, the ocular surface disorder is selected from the group consisting of dry eye disease, meibomian gland dysfunction and blepharitis.

In particular embodiments, the ocular surface disorder is dry eye disease.

In further particular embodiments, the ocular surface disorder is meibomian gland dysfunction.

In further particular embodiments, the ocular surface disorder is blepharitis.

In particular embodiments of the liposomes, compositions and compounds for use, methods and uses described herein, the treatment or prevention comprises topically administering a composition of the invention (or a composition comprising the liposomes of the invention or a compound of the invention) to the eye surface.

In further particular embodiments, the treatment of an ocular surface disorder comprises reducing the rate of evaporation or water from the eye surface.

In a further aspect, there is provided the use of a composition, liposome or compound of the invention in reducing or preventing the evaporation of water from a surface (such as the eye surface).

In a further aspect of the invention there is provided a method reducing and/or preventing evaporation of water from a surface (e.g. an eye surface comprising) applying a composition, liposome or compound of the invention (or a composition comprising a compound of the invention) to the surface.

In a further aspect there is provided a method of alleviating eye discomfort comprising administering a composition, liposome or compound of the invention to a patient in need thereof (in particular by topically administering the composition, liposome or compound to the eye surface of the patient).

In a further aspect, there is provided a composition, liposome or compound of the invention for use in alleviating eye discomfort, optionally wherein the use comprises topically administering the composition, liposome or compound to the eye surface.

In a further aspect, there is provided the use of a composition, liposome or compound of the invention in the manufacture of a medicament for alleviating eye discomfort, optionally wherein the composition, liposome or compound is administered topically to the eye surface to alleviate the discomfort.

### Preparation of Compositions and Compounds of the Invention

The compositions and liposomes of the invention may be prepared in accordance with techniques well known to the person skilled in the art. In particular, such compositions and liposomes may be prepared according to the process described in Example 9.

Accordingly, in a further aspect of the invention, there is provided a process for the preparation of the compositions and/or liposomes of the invention comprising the steps of:
1. Dissolving the lipid and phospholipid components (as defined herein) to form a solution containing the desired ratio of lipid and phospholipid components (as defined herein);
2. Heating the chloroform solution obtained in step 1 to the phase transition temperature for the composition;
3. Evaporating the chloroform to produce a thin lipid layer
4. Hydrating the solution with water; and, optionally
5. Sonicating the solution.

Compounds of the invention, and the compounds used in the compositions and liposomes of the invention as described herein may be prepared in accordance with techniques that are well known to those skilled in the art, such as those described in the examples provided hereinafter. Certain compounds used in the compositions and liposomes of the invention are also commercially available.

According to a further aspect of the invention there is provided a process for the preparation of a fatty acid ester of a hydroxy fatty acid or formula I, as defined herein, wherein X¹ represents

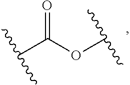 [SMILES: *OC(*)=O]

comprising reacting a compound of formula V

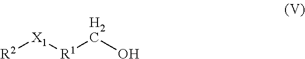

wherein X¹ represents

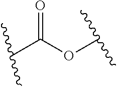 [SMILES: *OC(*)=O]

and R¹ and R² are as defined herein, as appropriate, with a suitable oxidizing agent, such as Jones reagent (chromium trioxide in diluted sulfuric acid) in the presence of a suitable solvent, such as tetrahydrofuran.

In a further aspect of the invention, there is provided a process for the preparation of a fatty acid ester of a hydroxy fatty acid or formula I, as defined herein, wherein X¹ represents

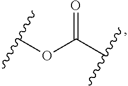 [SMILES: *OC(*)=O]

comprising reacting a compound of formula VI,

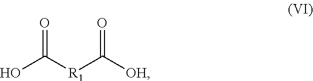

wherein R¹ is as defined herein, as appropriate, with a compound of formula VII

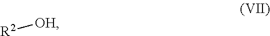

wherein R² is as defined herein, as appropriate, in the presence of a suitable acid or base, such as NaHSO₄) and a suitable solvent, such as water

Compounds of formulae V, VI and VII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

The skilled person will understand that the substituents as defined herein, and substituents thereon, may be modified one or more times, after or during the processes described above for the preparation of compounds of the invention by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention, and the compounds used in the compositions and liposomes of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999), the contents of which are incorporated herein by reference.

When used herein in relation to a specific value (such as an amount, or a ratio), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

### Further Embodiments

Further particular embodiments of the invention are provided in the following numbered Paragraphs.
Paragraph 1. A pharmaceutical composition comprising liposomes, which liposomes comprise:
(a) a lipid component selected from the group consisting of:
(i) a fatty acid ester of a hydroxy fatty acid of formula I or a pharmaceutically acceptable salt thereof,

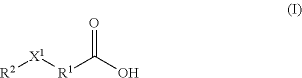

wherein:
R¹ is selected from a C₁₆-C₃₀ alkanediyl or a C₂₁-C₃₀ alkenediyl group containing one double bond;
R² is selected from a C₁₅-C₁₉ alkyl group or a C₁₅-C₁₉ alkenyl group containing one or two double bonds; and
X¹ represents an ester group selected from the group consisting of:

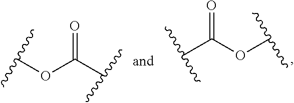

wherein [symbol] represents a point of attachment to the rest of the molecule;
(ii) a combination of a fatty acid ester of a hydroxy fatty acid of formula I, or a pharmaceutically acceptable salt thereof, as defined in (i), and a wax ester of formula II

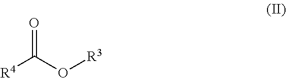

wherein:
R³ represents a C₁₈-C₃₀ alkyl group, and
R⁴ represents a C₁₀-C₂₄ alkyl group or a C₁₀-C₂₄ alkenyl group containing one or two double bonds;
wherein the fatty acid ester of a hydroxy fatty acid of formula I and the wax ester of formula II are present in a molar ratio of from about 2:1 to about 1:9;
(iii) a fatty alcohol of formula III, or a pharmaceutically acceptable salt thereof,

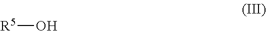

wherein R⁵ represents a C₁₄-C₃₀ linear or branched alkyl group; or formula IV,

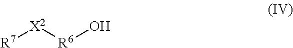

wherein:
R⁶ is selected from a C₁₀-C₃₀ alkanediyl or a C₁₀-C₃₀ alkenediyl group containing one double bond;
R⁷ is selected from a C₁₄-C₁₉ alkyl group or a C₁₄-C₁₉ alkenyl group containing one or two double bonds; and
X² represents an ester group selected from the group consisting of

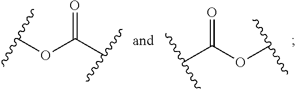 [SMILES: *OC(*)=O ; *OC(*)=O]

(iv) a combination of a fatty acid ester of a hydroxy fatty acid of formula I, or pharmaceutically-acceptable salt thereof with a fatty alcohol of formula III or IV, or a pharmaceutically acceptable salt thereof, wherein the components are present in an mass ratio of from about 3:1 to about 1:3; or
(v) a combination of a wax ester of formula II with a fatty alcohol of formula III or IV, or a pharmaceutically acceptable salt thereof, wherein the components are present in a mass ratio of from about 3:1 to about 1:3; and
(b) a phospholipid component, which is selected from the group consisting of a phosphatidylcholine, a phosphatidylglycerol, or a pharmaceutically acceptable salt thereof, a phosphatidylserine, or a pharmaceutically acceptable salt thereof, a phosphatidic acid, or pharmaceutically acceptable salt thereof, a phosphatidylethanolamine and a mixture thereof;
wherein the lipid component and phospholipid component are present, in the composition, in a ratio of from about 10:1 to about 1:10 by mass.
Paragraph 2. A composition as defined in Paragraph 1, wherein R¹ is a C₁₇-C₂₄ alkanediyl group or a C₂₄-C₂₉ alkenediyl group containing one double bond; optionally wherein R¹ is a C₁₇-C₂₂ alkanediyl group or a C₂₇-C₂₉ alkenediyl group containing one double bond.
Paragraph 3. A composition as defined in Paragraph 1, wherein R¹ is a C₁₇-C₁₉ linear alkanediyl group.
Paragraph 4. A composition as defined in any one of Paragraphs 1 to 3, wherein X¹ represents

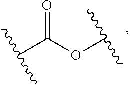 [SMILES: *OC(*)=O]

and/or R² represents a C₁₇ alkenyl group containing one or two double bonds, optionally wherein R² represents

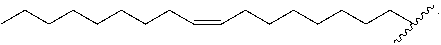 [SMILES: *CCCCCCC/C=C\CCCCCCCC]

Paragraph 5. A composition as defined in any one of Paragraphs 1 to 3, wherein X¹ represents

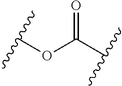 [SMILES: *OC(*)=O]

and/or
R² represents a C₁₈ alkenyl group containing one or two double bonds, preferably wherein R² represents

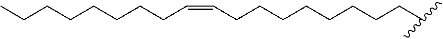 [SMILES: *CCCCCCCC/C=C\CCCCCCCC]

Paragraph 6. A composition as defined in any one of Paragraphs 1 to 5, wherein R³ represents a C₁₈ to C₂₆ alkyl group, preferably wherein R² represents a C₁₈ to C₂₂ alkyl group.
Paragraph 7. A composition as defined in any one of Paragraphs 1 to 5, wherein R⁴ represents a C₁₁ to C₂₂ alkyl or a C₁₁ to C₂₂ alkenyl group containing one or two double bonds.
Paragraph 8. A composition as defined in any one of Paragraphs 1 to 7, wherein R⁵ represents a C₁₆ to C₂₆ alkyl group, preferably wherein R⁵ represents a Cis alkyl group.
Paragraph 9. A composition as defined in any one of Paragraphs 1 to 8, wherein X² represents

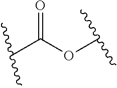 [SMILES: *OC(*)=O]

Paragraph 10. A composition as defined in any one of Paragraphs 1 to 9, wherein R⁶ represents a C₁₅-C₂₂ alkanediyl group, preferably wherein R⁶ represents a Cis to C₂₀ alkanediyl group.
Paragraph 11. A composition as defined in any one of Paragraphs 1 to 10, wherein R⁷ represents a C₁₇ or C₁₈ alkenyl group containing one double bond, preferably wherein X² represents

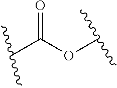 [SMILES: *OC(*)=O]

and R⁷ represents

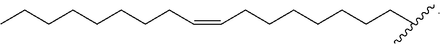 [SMILES: *CCCCCCC/C=C\CCCCCCCC]

Paragraph 12. A composition as defined in any one of Paragraphs 1 to 12, wherein the fatty acid ester of a hydroxy fatty acid of formula I (optionally the form of a pharmaceutically acceptable salt) is selected from the group consisting of:

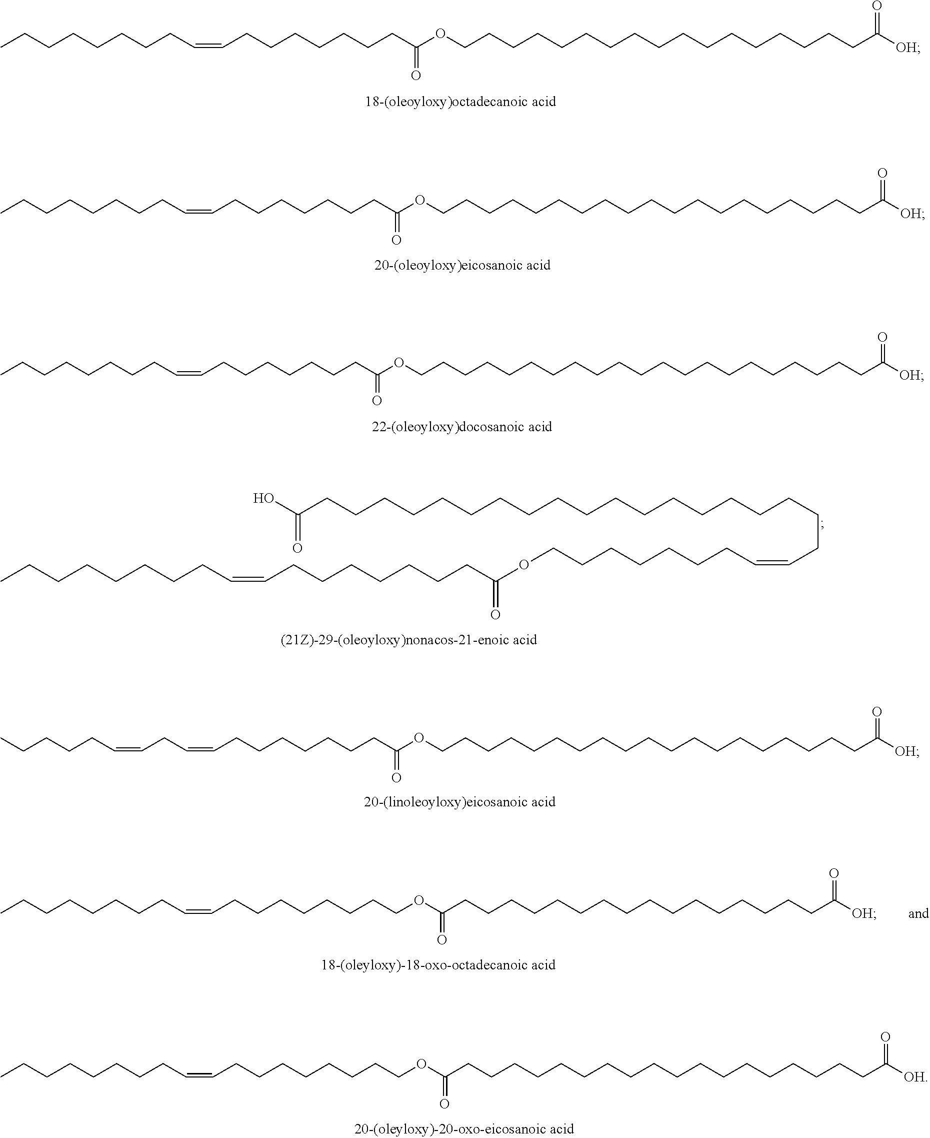

Paragraph 13. A composition as defined in Paragraph 12, wherein the fatty acid ester of a hydroxy fatty acid of formula I (optionally present in the form of a pharmaceutically acceptable salt) is selected from the group consisting of:

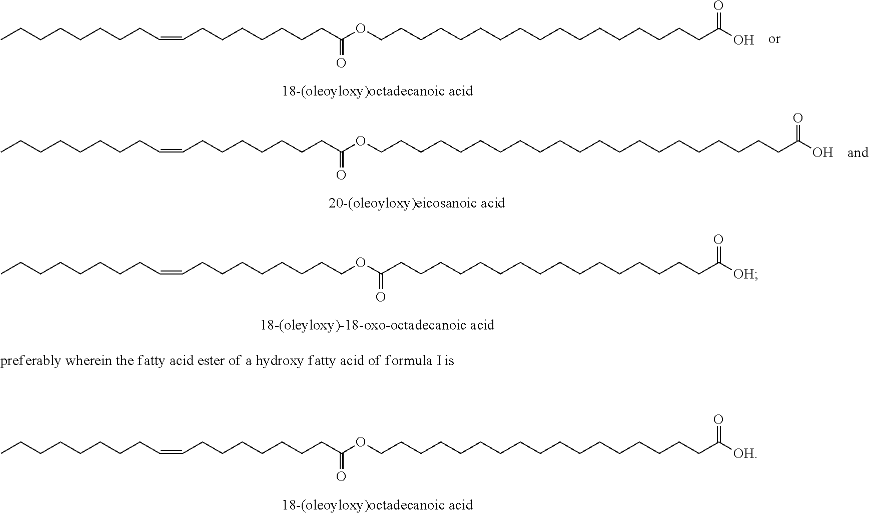 [SMILES: CCCCCCCC/C=C\CCCCCCCC(=O)OCCCCCCCCCCCCCCCCCC(=O)O ; "or" ; CCCCCCCC/C=C\CCCCCCCC(=O)OCCCCCCCCCCCCCCCCCCCCCC(=O)O ; "and" ; CCCCCCCC/C=C\CCCCCCCCOC(=O)CCCCCCCCCCCCCCCCC(=O)O ; CCCCCCCC/C=C\CCCCCCCC(=O)OCCCCCCCCCCCCCCCCCC(=O)O]

Paragraph 14. A composition as defined in any one of Paragraphs 1 to 13, wherein the wax ester of formula II is selected from the group consisting of:

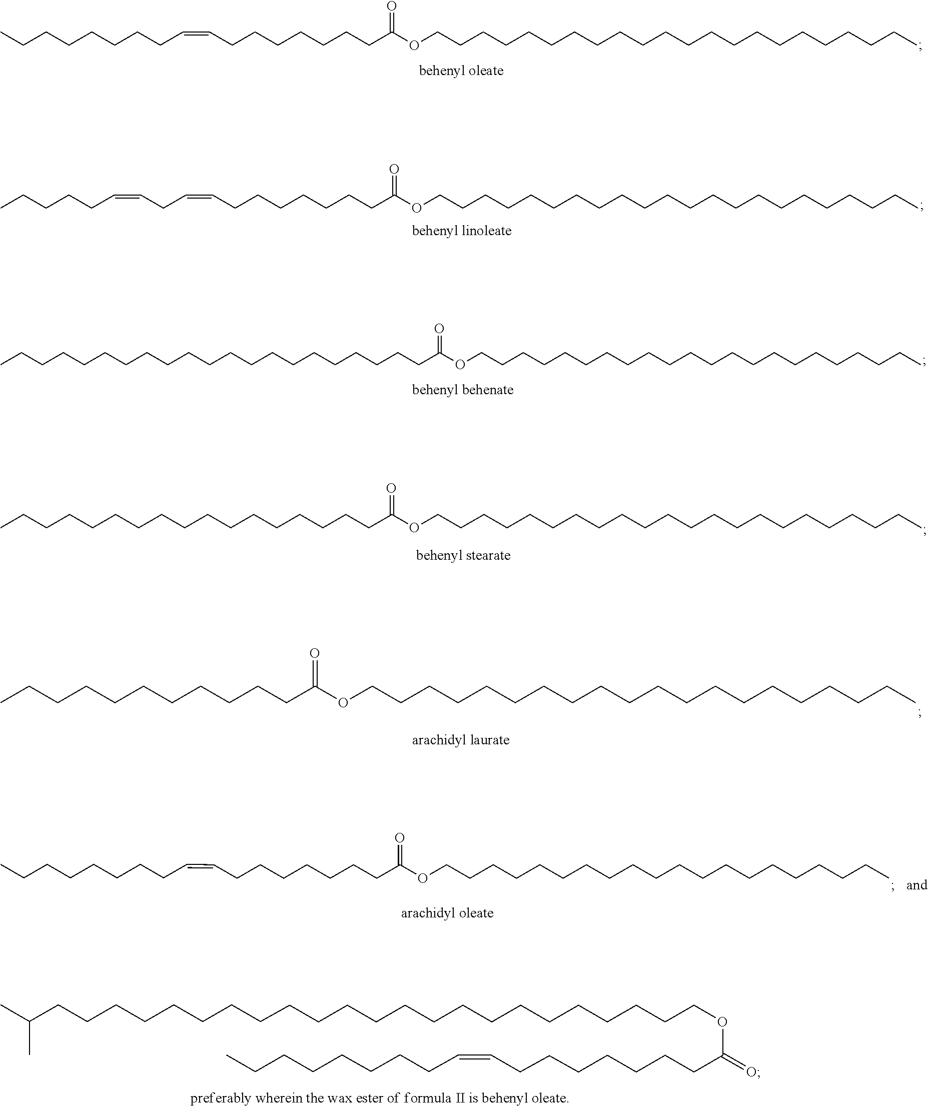

Paragraph15. A composition as defined in any one of the preceding numbered Paragraphs, wherein the fatty alcohol is of formula III and is octadecanol (which is optionally present in the form of a pharmaceutically acceptable salt).
Paragraph 16. A composition as defined in any one of the preceding numbered Paragraphs, wherein the lipid component is a fatty acid ester of a hydroxy fatty acid of formula I, or a pharmaceutically acceptable salt thereof, as defined in any one of the preceding claims.
Paragraph 17. A composition as defined in any one of the preceding numbered Paragraphs, wherein the lipid component is a combination of a fatty acid ester of a hydroxy fatty acid of formula I and a wax ester of formula II, wherein the fatty acid ester of a hydroxy fatty acid of formula I and the wax ester of formula II are present in a molar ratio of from about 1:1 to about 1:9, such as about 1:1; optionally wherein the lipid component is a selected from the group consisting of:
a combination of 20-(oleoyloxy) eicosanoic acid, or a pharmaceutically acceptable salt thereof, and behenyl oleate;
a combination of 20-(oleoyloxy) eicosanoic acid, or a pharmaceutically acceptable salt thereof, and arachidyl laurate;
a combination of 18-(oleoyloxy) octadecanoic acid, or a pharmaceutically acceptable salt thereof, and behenyl oleate;
a combination of 18-(oleoyloxy) octadecanoic acid, or a pharmaceutically acceptable salt thereof, and behenyl linoleate;
a combination of 18-(oleoyloxy) octadecanoic acid, or a pharmaceutically acceptable salt thereof, and behenyl behenate;
a combination of 18-(oleoyloxy) octadecanoic acid, or a pharmaceutically acceptable salt thereof, and behenyl stearate;
a combination of 18-(oleoyloxy) octadecanoic acid, or a pharmaceutically acceptable salt thereof, and arachidyl laurate;
a combination of 18-(oleyloxy)-18-oxo-octadecanoic acid, or a pharmaceutically acceptable salt thereof, and behenyl oleate;
a combination of (21Z)-29-(oleoyloxy) nonacos-21-enoic acid and 24-methylpentacosyl oleate
wherein, for each combination, the fatty acid ester of a hydroxy fatty acid and wax ester are present in a molar ratio of about 1:1.
Paragraph 18. A composition as defined in Paragraph 17, wherein the lipid component is selected from a combination of 18-(oleoyloxy) octadecanoic acid, or a pharmaceutically acceptable salt thereof, and behenyl oleate in a 1:1 molar ratio and a combination of 20-(oleoyloxy) eicosanoic acid, or a pharmaceutically acceptable salt thereof, and behenyl oleate in a 1:1 molar ratio.
Paragraph 19. A composition as defined in any one of Paragraphs 1 to 15, wherein the lipid component is a fatty alcohol of formula III or formula IV, or a pharmaceutically acceptable salt thereof, preferably wherein the lipid component is octadecanol, or a pharmaceutically acceptable salt thereof.
Paragraph 20. A composition as defined in any one of Paragraphs 1 to 15, wherein the lipid component is a combination of a fatty acid ester of a hydroxy fatty acid of formula I, or pharmaceutically-acceptable salt thereof, with a fatty alcohol of formula III or IV, or a pharmaceutically acceptable salt thereof, wherein the components are present in mass ratio of from about 3:1 to about 1:3, such as a mass ratio of from about 2:1 to about 1:2, such as about 1:1
Paragraph 21. A composition as defined in Paragraph 20, wherein the fatty acid ester of a hydroxy fatty acid is 18-(oleoyloxy) octadecanoic acid and the fatty alcohol is 1-octadecanol.
Paragraph 22. A composition as defined in any one of Paragraphs 1 to 15, wherein the lipid component is a combination of a wax ester of formula II with a fatty alcohol of formula III or IV, or a pharmaceutically acceptable salt thereof, wherein the components are present in a mass ratio from about 3:1 to about 1:3, such as a mass ratio of from about 2:1 to about 1:2, such as about 1:1.
Paragraph 23. A composition as defined in Paragraph 22, wherein the wax ester is behenyl oleate and the fatty alcohol is 1-octadecanol, or a pharmaceutically acceptable salt thereof.
Paragraph 24. A composition as defined in any one of the preceding numbered Paragraphs, wherein the phospholipid component is selected from the group consisting of 1,2-dimyristoyl-sn-glycero-3-phosphocholine (DMPC),,2-dipentadecanoyl-sn-glycero-3-phosphocholine, dipalmitoylphosphatidyl choline (DPPC), 1,2-distearoyl-sn-glycero-3-phosphocholine (DSPC), 1,2-diarachidoyl-sn-glycero-3-phosphatidylcholine (DAPC), 1,2-dioleoyl-sn-glycero-3-phosphocholine (DOPC), and mixtures thereof.
Paragraph 25. A composition as defined in Paragraph 24, wherein the phospholipid component is selected from the group consisting of 1,2-distearoyl-sn-glycero-3-phosphocholine (DSPC), 1,2-diarachidoyl-sn-glycero-3-phosphatidylcholine (DAPC), 1,2-dioleoyl-sn-glycero-3-phosphocholine (DOPC), and mixtures thereof; preferably wherein the phospholipid component is selected from the group consisting of 1,2-distearoyl-sn-glycero-3-phosphocholine (DSPC), 1,2-diarachidoyl-sn-glycero-3-phosphatidylcholine (DAPC), and mixtures thereof.
Paragraph 26. A composition as defined in any one of Paragraphs 1 to 25, wherein the lipid component and phospholipid component are present, in the composition, in a ratio of about 2:1 to about 1:10 by mass, preferably in a ratio of from about 1:1 to 1:4 by mass.
Paragraph 27. A composition as defined in any one of Paragraphs 1 to 26, wherein at least about 80% by mass of the lipid and phospholipid components are incorporated into the liposomes, optionally wherein at least about 90% by mass of the lipid and phospholipid components are incorporated into the liposomes and preferably wherein at least about 95% by mass of the lipid and phospholipid components are incorporated into the liposomes.
Paragraph 28. A liposome comprising a lipid component and a phospholipid component as defined in any one of the preceding claims in a ratio of from about 10:1 to about 1:10 by mass, optionally from about 2:1 to about 1:10 by mass, such as from about 1:1 to about 1:8 by mass, for example in a ratio of from about 1:1 to 1:4 by mass, such as in a ratio of about 1:1-1:3 by mass.
Paragraph 29. A pharmaceutical composition comprising a liposome as defined in Paragraph 28.
Paragraph 30. A pharmaceutical composition as defined in any one of Paragraphs 1 to 27 and 29, wherein the combined amount of the lipid and phospholipid components in the composition is from about 0.1% (w/v) to about 20% (w/v) of the composition.
Paragraph 31. A pharmaceutical composition as defined in any one of claims 1 to 27 and 29, wherein lipid component of the liposome or composition, as defined in any one of the preceding claims, the is present, in the composition, in an amount of from about 0.1 (w/v) to about 10% (w/v), optionally from about 0.5% (w/v) to about 8% (w/v) or from about 0.5% (w/v) to about 5% (w/v) or from about 0.5% to about 2% (w/v)).
Paragraph 32. A pharmaceutical composition as claimed in any one of Paragraphs 1 to 27 and 29, wherein the phospholipid compound, as defined in any one of the preceding claims, is present, in the composition, in an amount of from about 0.1% (w/v) to about 10% (w/v), optionally from about 0.5% (w/v) to about 8% (w/v) or from about 0.5% (w/v) to about 5% (w/v) or from about 0.5% to about 2% (w/v)).
Paragraph 33. A compound selected from the group consisting of
15-(oleyloxy)-15-oxo-pentadecanoic acid
18-(oleyloxy)-18-oxo-octadecanoic acid
20-(oleyloxy)-20-oxo-eicosanoic acid,
22-(palmitoleyloxy)-22-oxo-behenic acid
20-(palmitoleyloxy)-20-oxo-eicosanoic acid; and
18-(palmitoleyloxy)-18-oxo-octadecanoic acid
or a pharmaceutically acceptable salt thereof.
Paragraph 34. A pharmaceutical composition comprising a compound as defined in Paragraph 33, or a pharmaceutically acceptable salt thereof.
Paragraph 35. A liposome, composition or compound as defined in any one of the preceding numbered Paragraphs for use in the treatment and/or prevention of an ocular surface disorder or allergic conjunctivitis.
Paragraph 36. A method for treating and/or preventing an ocular surface disorder or allergic conjunctivitis, comprising administering to a patient in need thereof a therapeutically effective amount of a liposome, composition or compound as defined in any one of the preceding numbered Paragraphs to a patient in need thereof.
Paragraph 37. The use of a liposome, composition or compound as defined in any one of the preceding numbered Paragraphs in the manufacture of a medicament for the treatment and/or prevention of an ocular surface disorder or allergic conjunctivitis.
Paragraph 38. The liposome, composition or compound for use, method or use as defined in any one of Paragraphs 35 to 37, wherein the ocular surface disorder is selected from the group consisting of dry eye disease, Meibomian gland dysfunction and blepharitis.
Paragraph 39. The liposome, composition or compound for use, method or use as defined in any one of Paragraphs 35 to 38, wherein the treatment and/or prevention comprises topically administering the liposome, compound or composition to the eye surface.

Without wishing to be bound by theory, it is believed that the compositions, liposomes and compounds described herein offer benefits compared to similar species known in the prior art. In particular, the liposomes and formulations comprising the same provide treatments for ocular surface disorders, such as dry eye disease and Meibomian gland dysfunction with improved evaporation resistance compared to known similar treatments (such as commercially available eye drop solutions). The liposomes of the invention and compositions comprising the same further allow the preparation of stable formulations of lipophilic components, such as fatty acid esters of hydroxy fatty acids, wax esters and fatty alcohols that also allow efficient spreading of these species at the eye surface and formation of an anti-evaporative film. Together these two properties are uniquely suited for targeting the tear film instability defect associated with ocular surface disorders such as dry eye disease and Meibomian gland dysfunction.

### BRIEF DESCRIPTION OF THE FIGURES

FIG. 1A shows surface pressure isotherms of 20:1/18:1-OAHFA and 18:0/18:1-OAHFA as a function of the mean molecular area. The numbers (i-vi) correspond to the BAM-images in FIG. 1B. FIG. 1B BAM-images showcasing the visual appearance of the film at selected mean molecular areas. The 20:1/18:1-OAHFA is in a liquid state throughout the surface pressure range studies whereas the 18:0/18:1-OAHFA undergoes a liquid to solid phase transition and is solid at the ocular surface pressure. The scale bar depicts 500 μm in the BAM images.

FIG. 2 shows the evaporation-resistant properties of different OAHFAs expressed as percentages compared to the degree of evaporation in the absence of a lipid film determined using the procedure described in Example 10. The figure shows the deviating evaporation resistance of a range of OAHFAs containing variable chain lengths and saturation degrees.

FIG. 3A shows surface pressure isotherms of 29:1/18:1-OAHFA and mixtures of 29:1/18:1-OAHFA and Iso-WE (1:1, 1:9) as a function of the mean molecular area. The numbers (I-V) correlate with the BAM-images in FIG. 3C. FIG. 3B shows surface potential isotherms of 29:1/18:1-OAHFA and mixtures of 29:1/18:1-OAHFA and Iso-WE (1:1, 1:9) as a function of the mean molecular area. FIG. 3C BAM-images showcasing the visual appearance of the film at selected mean molecular areas. The surface pressure and surface potential isotherms show how the behavior of the mixtures change depending on the ratio between the OAHFA and WE. A 1:1-mixture displays a similar behavior as the individual OAHFA. The BAM-images showcase the properties of the films at selected surface pressures. BAM-image I displays a mixture in liquid state. BAM-image II displays a heterogeneous phase containing both a liquid state and a solid state. BAM-images III-V display a solid phase with an increase in packing density as function of increased surface pressure. The scale bar depicts 500 μm in the BAM images.

FIG. 4 shows the evaporation-resistant properties of different 1:1 mixtures of OAHFAs and wax esters expressed as percentages compared to the degree of evaporation in the absence of a lipid film determined using the procedure described in Example 10.

FIG. 5 shows the evaporation-resistant properties of different long chain fatty alcohols expressed as percentages compared to the degree of evaporation in the absence of a lipid film determined using the procedure described in Example 10.

FIG. 6 shows the evaporation resistant properties of certain mixtures of the evaporation-resistant active ingredients with phospholipids expressed as percentages compared to the degree of evaporation in the absence of a lipid film determined using the procedure described in Example 10.

FIG. 7A shows DSC thermograms of different liposomal formulations containing DMPC in a mass ratio of 4:1 (DMPC: active evaporation-resistant components). Line 1 shows the thermogram for DMPC alone, line 2 shows the thermogram for DMPC: 18:0/18:1-OAHFA (4:1 (mass ratio)), line 3 shows the thermogram for DMPC: 18:0/18:1-OAHFA:BO (8:1:1 (mass ratio)) and line 4 shows the thermogram for DMPC:octadecanol: 18:0/18:1-OAHFA (8:1:1 (mass ratio)).

FIG. 7B shows DSC thermograms of DSC thermograms of different liposomal formulations containing DSPC in a mass ratio of 2:1 DSPC:active evaporation resistant components. Line 1 shows the thermogram for DSPC alone, line 2 shows the thermogram for DSPC: 18:0/18:1-OAHFA (2:1 (mass ratio)), line 3 shows the thermogram for DSPC: 18:0/18:1-OAHFA:BO (4:1:1 (mass ratio)) and line 4 shows DSPC:octadecanol: 18:0/18:1-OAHFA (4:1:1 (mass ratio)).

FIG. 7C shows DSC thermograms of different liposomal formulations containing DAPC in a mass ratio of 2:1 DAPC:active evaporation resistant components. Line 1 shows the thermogram for DAPC alone, line 2 shows the thermogram for DAPC: 18:0/18:1-OAHFA (2:1 (mass ratio)), line 3 shows the thermogram for DAPC: 18:0/18:1-OAHFA:BO (4:1:1 (mass ratio)) and line 4 shows DAPC:octadecanol: 18:0/18:1-OAHFA (4:1:1 (mass ratio)).

FIG. 8 shows the transformations occurring in the surface pressure isotherms over compression-expansion cycles of selected liposomal formulations (A to L) as a function of the area. The volume added of each formulation is indicated.

FIG. 9A. shows surface pressure isotherm over a single compression-expansion cycle as a function of area for a liposomal formulation comprising 2% DAPC and 1% 18:0/18:1-OAHFA (18-OAHFA). The numbers (I-VI) correlate with the BAM-images in FIG. 9B. FIG. 9B shows BAM-images showcasing the visual appearance of the film at selected areas and surface pressures. At low surface pressures (image I), the film exists in the liquid state. A gradual increase in the surface pressure is accompanied by a liquid to solid phase transition and the film exists in the solid phase at ocular surface pressures (III-V). The scale bar depicts 500 μm in the BAM images.

FIG. 10 shows the evaporation resistance of different liposomal formulations expressed as percentages compared to the degree of evaporation in the absence of a lipid film determined using the procedure described in Example 10.

FIG. 11 shows the viability %±SD of HCE cells after 3-hour formulation exposure (½ formulation dilution with serum-free media) to the liposomal compositions of the invention and the commercial formulations Oxyal® triple action and Systane® complete. Viability was determined either straight after formulation exposure (dark grey bars), or after allowing the cells to recover overnight (light grey bars). Control cells that were not exposed to formulations denoted the reference level of 100% cell viability.

FIG. 12 shows the results of a model study using the benzalkonium chloride (BAC) model of dry eye disease. Viability % (+SD) of the HCE cells 24 hours after BAC exposure and with 24-hour formulation treatment (¼ dilution with serum-free media) is shown. Control cells that were not exposed to BAC nor formulations denoted the reference level of 100% cell viability, while the cells that were only exposed to BAC but not treated with formulation provided a baseline level (negative control), and the cells that were exposed to BAC and treated with full growth medium containing 15% FBS served a positive control for cell recovery.

FIG. 13A shows surface pressure isotherms of the phospholipids 1,2-distearoyl-sn-glycero-3-phospho-rac-(1-glycerol) sodium salt (18:0 PG), 1,2-dipalmitoyl-sn-glycero-3-phosphoethanolamine (16:0 PE) and 1,2-distearoyl-sn-glycero-3-phosphoethanolamine (18:0 PE). The Roman numerals I to IX correspond to the BAM images shown in FIG. 13B (images I, II and III show the film structure of 18:0 PG at different pressures, images IV, V and VI show the film structure of 16:0 PE at different pressures and images VII, VIII and IX show the film structure of 18:0 PE at different pressures). FIG. 13C shows the evaporation resistance (s/cm) for 16:0 PE, 18:0 PE, 18:0 PG and 1,2-distearoyl-sn-glycero-3-phospho-L-serine (sodium salt) (18:0 PS).

FIG. 14 shows the evaporation reduction (expressed as percentages compared to the degree of evaporation in the absence of a lipid film determined using the procedure described in Example 10) achieved by the following 1:1:1 (phospholipid:OAHFA:Wax ester) combinations: DAPC:20-OAHFA:BO, 14:0-PG: 20-OAHFA:BO; 18:0-PG: 20-OAHFA:BO; 16:0-PE:20-OAHFA:BO; 18:0-PE:20-OAHFA:BO. Of these combinations, the DAPC:20-OAHFA:BO combination achieved by far the highest level of evaporation reduction.

FIG. 15A shows surface pressure isotherms for the commercial products BevitaEye, Evotears and TheaLipid. The numbers I, II and III correspond to the BAM images in FIG. 15B. FIG. 15C shows the evaporation reduction (expressed as percentages compared to the degree of evaporation in the absence of a lipid film determined using the procedure described in Example 10) achieved by a liposomal formulation comprising 2% DAPC, 1% 20-OAHFA and 1% behenyl oleate, as described herein (labelled as Invention), compared to a range of commercially available products. The graph shows that the composition of the invention achieved significantly higher evaporation reduction that the commercial products.

### EXAMPLES

The present invention will be further described by reference to the following examples, which are not intended to limit the scope of the invention.

### Synthesis and Structural Characterization of Active Components

All reagents were purchased from commercial sources. Dry solvents were purified by the VAC vacuum solvent purification system prior to use when dry solvents were needed. All reactions containing moisture- or air-sensitive reagents were carried out under an argon atmosphere. All reactions requiring heating were performed using an oil bath. Thin-layer chromatography (TLC) was performed on aluminium sheets pre-coated with silica gel 60 F254 (Merck). Flash chromatography was carried out using silica gel 40. Spots were visualized by UV followed by spraying with 1:4 H₂SO₄/MeOH-solution and heating. HRMS were recorded using a Bruker Micro Q-TOF with ESI (electrospray ionization) operated in positive mode or a Bruker Ultraflex III mass spectrometer operated in positive mode. NMR spectra were recorded with a Bruker Avance III NMR spectrometer operating at 500.13 MHz (¹H) or 499.82 MHz (¹H) and 125.68 MHz (¹³C). All products were characterized by a combination of 1D (¹H and ¹³C) and 2D techniques (DQF-COSY, Ed-HSQC and HMBC) with pulse sequences provided by the instrument manufacturer. The probe temperature was kept at 25° C. unless otherwise stated. The chemical shifts are expressed on the 0 scale (in ppm) using TMS (tetramethylsilane) or residual chloroform as internal standards. Melting point analysis was performed using a Mettler Toledo MP50 (Columbus, OH, USA) melting point system for end products when possible.

### Synthesis of OAHFAs, Long-Chain Fatty Alcohols and Reversed OAHFAS (R-OAHFAS)

A number of long-chain fatty alcohols are commercially available. Moreover, during the synthesis of OAHFAs, long-chain fatty alcohols are produced during the synthesis routes. The synthesis of long-chain fatty alcohols and OAHFAs will be exemplified by the synthesis of the following oleic acid derivatives: 18:0/18:1-OAHFA (and corresponding alcohol), 20:1/18:1-OAHFA (and corresponding alcohol) and 29:1/18:1-OAHFA (and corresponding alcohol). The synthesis of reversed OAHFAs (R-OAHFAs) will be exemplified by the synthesis of R-18:0/18:1-OAHFA ((Z)-18-(oleyloxy)-18-oxo-octadecanoic acid). The characterization data for other long-chain fatty alcohols, OAHFAs and R-OAHFAs prepared through similar techniques will be provided at the end of each relevant section.

### Example 1. Synthesis of 18-(Oleoyloxy) Octadecanoic Acid (Corresponding Alcohol) and Other OAHFAs (Alcohols) Accessed Through a Similar Synthesis Route and in Similar Yields

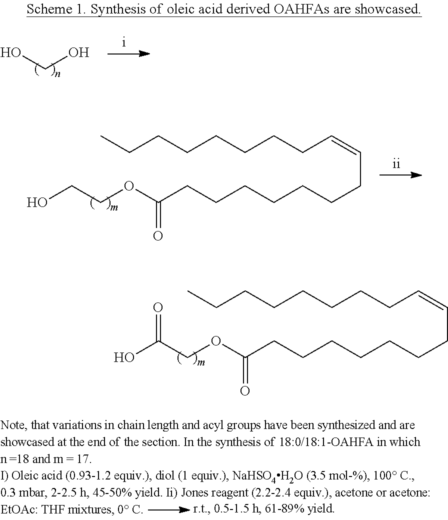

18-(oleoyloxy) octadecan-1-ol (18:0/18:1-OAHFAI). Oleic acid (1.29 g, 4.6 mmol, 0.93 equiv.), NaHSO₄·H₂O (0.024 g, 3.5 mol-%) and 1,18-Octadecanediol (1.40 g, 4.9 mmol, 1 equiv.) were added to a round bottom flask (100 ml). The stirred mixture was heated to 100° C. on an oil bath under vacuum. After 2 hours, the reaction was brought to r.t., diluted with CHCl₃ (50 ml) and washed with saturated NaHCO₃ (2×50 ml). The aqueous phase was re-extracted with CHCl₃ (2×50 ml) and the organic layers were combined and washed with brine (80 ml), dried over Na₂SO₄, filtered and concentrated. The crude product was purified by column chromatography (Hexane:EtOAc 19:1-4:1), concentrated and dried on the vacuum line to give the title compound as a white solid (1.21 g, 45% yield). Mp 51.9-53.0° C. ¹H NMR (499.82 MHz, CDCl₃, 25° C.): δ 5.39-5.30 (m, 2H), 4.05 (t, 2H), 3.66-3.60 (m, 2H), 2.28 (t, 2H), 2.04-1.97 (m, 4H), 1.66-1.52 (m, 6H), 1.38-1.21 (m, 49H) and 0.88 (t, 3H) ppm. ¹³C NMR (125.68 MHz, CDCl₃, 25° C.): δ 174.1, 130.1, 129.9, 64.6, 63.3, 34.6, 33.0, 32.1, 29.9-29.3, 28.8, 27.4, 27.3, 26.1, 25.9, 25.2, 22.8, 14.3 ppm. HRMS m/z calculated for C₃₆H₇₀O₃Na [M+Na]⁺ 573.5225, found 573.5141.

18-(oleoyloxy) octadecanoic acid (18:0/18:1-OAHFA). A solution of 18-hydroxyoctadecyl oleate (1.0818 g, 2.0 mmol, 1 equiv.) in THF:acetone:EtOAc (40 ml; 2:2:1 ratio) under argon atmosphere was cooled to 0° C. and Jones reagent (2.3 ml, 4.5 mmol, 2.3 equiv.) was added dropwise. The reaction mixture was stirred for 1.75 hours, quenched with 2-propanol (10 ml) and filtered through a pad of celite. The celite was then washed with Et₂O (250 ml) and the collected filtrate washed with Brine (100 ml), dried over Na₂SO₄, filtered and concentrated. The crude product was purified using column chromatography (Hexane:EtOAc:AcOH 19:1:0.01-7:3:0.01) and dried on the vacuum line to give the title compound as a white solid (0.9189 g, 83% yield). Mp 55.8-56.7° C.¹H NMR (499.82 MHz, CDCl₃, 25° C.): δ 5.39-5.29 (m, 2H), 4.05 (t, 2H), 2.34 (t, 2H), 2.29 (t, 2H), 2.06-1.97 (m, 4H), 1.67-1.56 (m, 6H), 1.37-1.21 (m, 46H) and 0.88 (t, 3H) ppm. ¹³C NMR (125.68 MHz, CDCl₃, 25° C.): δ 179.5, 174.2, 130.1, 130.0, 64.6, 34.6, 34.1, 32.1, 29.9-29.2, 28.8, 27.4, 27.3, 26.1, 25.2, 24.8, 22.8 and 14.3 ppm. HRMS m/z calculated for C₃₆H₆₈O₄Na [M+Na]⁺ 587.5015, found 587.5030.

The following compounds (OAHFAs and OAHFAIs) were prepared by analogous synthetic sequences.

### OAHFAIS

12-(oleoyloxy) dodecan-1-ol (12:0/18:1-OAHFAI). Mp rt. ¹H NMR (500.13 MHz, CDCl₃, 25° C.): δ 5.38-5.31 (m, 2H), 4.05 (t, 2H), 3.66-3.62 (m, 2H), 2.29 (t, 2H), 2.03-1.99 (m, 4H), 1.64-1.54 (m, 6H), 1.33-1.27 (m, 37H) and 0.88 (t, 3H) ppm. ¹³C NMR (125.68 MHz, CDCl₃, 25° C.): δ 174.2, 130.2, 129.9, 64.6, 63.3, 34.6, 33.0, 32.1-29.2, 28.8, 27.4, 27.3, 26.1, 25.9, 25.2, 22.8 and 14.3 ppm. HRMS m/z calculated for C₃₀H₅₈O₃Na [M+Na]⁺ 489.4278, found 489.4237.

12-(palmitoleoyloxy) dodecan-1-ol (12:0/16:1-OAHFAI). Mp 33.4° C. ¹H NMR (499.82 MHz, CDCl, 25° C.): δ 5.35 (dtt, 1H), 5.34 (dtt, 1H), 4.05 (t, 2H), 3.64 (t, 2H), 2.29 (t, 2H), 2.01 (ddt, 2H), 2.01 (ddt, 2H), 1.62 (tt, 2H), 1.61 (tt, 2H), 1.56 (tt, 2H), 1.38-1.20 (m, 32H) and 0.88 (t, 3H) ppm.¹³C NMR (125.68 MHz, CDCl₃, 25° C.): δ 174.1, 130.1, 129.9, 64.5, 63.2, 34.5, 33.0, 31.9, 29.9-28.8, 27.4-27.3, 26.1, 25.9, 25.2, 22.8 and 14.2 ppm. HRMS m/z calculated for C₂₈H₅₄O₃Na [M+Na]⁺ 461.3965, found 461.4001.

12-(palmitoyloxy) dodecan-1-ol (12:0/16:0-OAHFAI). Mp 63.3° C. ¹H NMR (499.82 MHz, CDCl₃, 25° C.): δ 4.05 (t, 2H), 3.64 (dt, 2H), 2.29 (t, 2H), 1.62 (tt, 2H), 1.60 (tt, 2H), 1.57 (tt, 2H), 1.38-1.20 (m, 41H) and 0.88 (t, 3H) ppm. ¹³C NMR (125.68 MHz, CDCl₃, 25° C.): δ 174.2, 64.5, 63.2, 34.6, 33.0, 32.1, 29.8-29.3, 28.8, 26.1, 26.0, 25.2, 22.8 and 14.3 ppm. HRMS m/z calculated for C₂₈H₅₆O₃Na [M+Na]⁺ 463.4122, found 463.4109. 12-(stearoyloxy) dodecan-1-ol (12:0/18:0-OAHFAI). Mp 66.6° C. ¹H NMR (499.82 MHz, CDCl₃, 25° C.): δ 4.05 (t, 2H), 3.64 (dt, 2H), 2.29 (t, 2H), 1.62 (tt, 2H), 1.60 (tt, 2H), 1.56 (tt, 2H), 1.38-1.22 (m, 44H), 1.21 (t, 1H) and 0.88 (t, 3H) ppm. ¹³C NMR (125.68 MHz, CDCl₃, 25° C.): δ 174.2, 64.5, 63.2, 34.6, 33.0, 32.1, 29.8-29.3, 28.8, 26.1, 26.0, 25.2, 22.8 and 14.3 ppm. HRMS m/z calculated for C₃₀H₆₀O₃Na [M+Na]⁺ 491.4406, found 491.4406.

12-(linoleoyloxy) dodecan-1-ol (12:0/18:2-OAHFAI). Mp 26.0° C. ¹H NMR (499.82 MHz, CDCl₃, 25° C.): δ 5.38 (dtt, 1H), 5.37 (dtt, 1H), 5.33 (dtt, 1H), 5.33 (dtt, 1H), 4.05 (t, 2H), 3.64 (dt, 2H), 2.77 (dddd, 2H), 2.28 (t, 2H), 2.05 (ddt, 2H), 2.05 (ddt, 2H), 1.62 (tt, 2H), 1.61 (tt, 2H), 1.56 (tt, 2H), 1.39-1.22 (m, 31H) and 0.88 (t, 3H) ppm. ¹³C NMR (125.68 MHz, CDCl₃, 25° C.): δ 174.1, 130.3, 130.2, 128.2, 128.0, 64.5, 63.2, 34.5, 32.9, 31.7, 29.7-29.3, 28.8, 27.3, 26.1, 25.9, 25.8, 25.1, 22.7 and 14.2 ppm. HRMS m/z calculated for C₃₀H₅₆O₃Na [M+Na]⁺ 487.4122, found 487.4111.

15-(oleoyloxy) pentadecan-1-ol (15:0/18:1-OAHFAI). Mp 42° C. ¹H NMR (500.13 MHz, CDCl₃, 25° C.): δ 5.38-5.31 (m, 2H), 4.05 (t, 2H), 3.65-3.64 (m, 2H), 2.29 (t, 2H), 2.03-1.99 (m, 4H), 1.64-1.54 (m, 6H), 1.30-1.27 (m 43H) and 0.88 (t, 3H) ppm. ¹³C NMR (125.68 MHz, CDCl₃, 25° C.): δ 174.2, 130.2, 129.9, 64.6, 63.3, 34.6, 33.0, 32.1, 29.9-29.2, 28.8, 27.4, 27.3, 26.1, 25.9, 25.2, 22.8 and 14.3 ppm. HRMS m/z calculated for C₃₃H₆₄O₃Na [M+Na]⁺ 531.4753, found 531.4712.

20-(stearoyloxy)eicosan-1-ol (20:0/18:0-OAHFAI). ¹H NMR (500.13 MHz, CDCl₃, 25° C.): δ 4.05 (t, 2H), 3.64 (dt, 2H), 2.29 (t, 2H), 1.62 (tt, 2H), 1.60 (tt, 2H), 1.56 (tt, 2H), 1.38-1.22 (m, 60H), 1.20 (t, 1H) and 0.88 (t, 3H) ppm.

¹³C NMR (125.68 MHz, CDCl₃, 25° C.): δ 174.2, 64.5, 63.1, 34.4, 32.8, 31.9, 29.7-29.2, 28.7, 26.0, 25.8, 25.1, 22.7 and 14.1 ppm. HRMS m/z calculated for C₃₈H₇₆O₃ [M+H]⁺ 581.5867, found 581.5674.

20-(oleoyloxy)eicosan-1-ol (20:0/18:1-OAHFAI). Mp 59° C. ¹H NMR (500.13 MHz, CDCl₃, 25° C.): δ 5.38-5.30 (m, 2H), 4.05 (t, 2H), 3.66-3.62 (m, 2H), 2.29 (t, 2H), 2.03-1.99 (m, 4H), 1.63-1.54 (m, 6H), 1.33-1.25 (m, 53H) and 0.88 (t, 3H) ppm. ¹³C-NMR (125.68 MHz, CDCl₃, 25° C.): δ 174.2, 130.2, 129.9, 64.6, 63.3, 34.6, 33.0, 32.1, 29.9-29.2, 28.8, 27.4, 27.3, 26.1, 25.9, 25.2, 22.2, 22.8 and 14.3 ppm. HRMS m/z calculated for C₃₈H₇₄O₃ [M+H]⁺ 579.5716, found 579.5713.

20-(linoleoyloxy)eicosan-1-ol (20:0/18:2-OAHFAI). Mp 74.0° C. ¹H NMR (499.82 MHz, CDCl₃, 25° C.): δ 5.38 (dtt, 1H), 5.37 (dtt, 1H), 5.33 (dtt, 1H), 5.33 (dtt, 1H), 4.05 (t, 2H), 3.64 (dt, 2H), 2.77 (dddd, 2H), 2.28 (t, 2H), 2.05 (ddt, 2H), 2.05 (ddt, 2H), 1.62 (tt, 2H), 1.61 (tt, 2H), 1.56 (tt, 2H), 1.39-1.22 (m, 46H), 1.22 (t, 1H) and 0.88 (t, 3H) ppm. ¹³C NMR (125.68 MHz, CDCl₃, 25° C.): δ 174.1, 130.4, 130.2, 128.2, 128.1, 64.6, 63.2, 34.6, 33.0, 31.7, 29.8-29.3, 28.8, 27.3, 26.1, 25.9, 25.2, 24.9, 22.7 and 14.2 ppm. HRMS m/z calculated for C₃₈H₇₂O₃Na [M+Na]⁺ 599.5374, found 599.5357.

20-(palmitoleoyloxy)eicosan-1-ol (20:0/16:1-OAHFAI). Mp 57.0° C. ¹H NMR (499.82 MHz, CDCl₃, 25° C.): δ 5.35 (dtt, 1H), 5.34 (dtt, 1H), 4.05 (t, 2H), 3.64 (t, 2H), 2.29 (t, 2H), 2.01 (ddt, 2H), 2.01 (ddt, 2H), 1.62 (tt, 2H), 1.60 (tt, 2H), 1.57 (tt, 2H), 1.38-1.20 (m, 48H) and 0.88 (t, 3H) ppm. ¹³C NMR (125.68 MHz, CDCl₃, 25° C.): δ 174.1, 130.1, 129.9, 64.5, 63.2, 34.5, 33.0, 31.9, 29.9-29.2, 28.8, 27.4-27.3, 26.1, 25.9, 25.2, 22.8 and 14.2 ppm. HRMS m/z calculated for C₃₆H₇₀O₃Na [M+Na]⁺ 573.5217, found 573.5171.

20-(palmitoyloxy)eicosan-1-ol (20:0/16:0-OAHFAI). Mp 52.9° C. ¹H NMR (499.82 MHz, CDCl₃, 25° C.): δ 4.05 (t, 2H), 3.64 (t, 2H), 2.29 (t, 2H), 1.61 (tt, 2H), 1.61 (tt, 2H), 1.57 (tt, 2H), 1.38-1.20 (m, 56H) and 0.88 (t, 3H) ppm.¹³C NMR (125.68 MHz, CDCl₃, 25° C.): δ 174.2, 64.6, 63.3, 34.6, 33.0, 32.1, 29.8-29.3, 28.8, 26.1, 25.9, 25.2, 22.8 and 14.3 ppm. HRMS m/z calculated for C₃₆H₇₂O₃Na [M+Na]⁺ 575.5374, found 575.5346.

22-(oleoyloxy)docosan-1-ol (22:0/18:1-OAHFAI). ¹H NMR (499.82 MHz, CDCl₃, 25° C.): δ 5.41-5.29 (m, 2H), 4.05 (t, 2H), 3.64 (t, 2H), 2.29 (t, 2H), 2.04-1.97 (m, 4H), 1.65-1.53 (m, 6H), 1.33-1.25 (m, 56H) and 0.88 (t, 3H) ppm. ¹³C-NMR (125.68 MHz, CDCl₃, 25° C.): δ 174.1, 130.1, 129.9, 64.6, 63.2, 34.6, 33.0, 32.1, 29.9-29.2, 28.8, 27.4, 27.3, 26.1, 25.9, 25.2, 22.2, 22.8 and 14.3 ppm. HRMS m/z calculated for C₄₀H₇₈O₃Na [M+Na]⁺ 629.5843, found 629.6350.

### OAHFAS

12-(oleoyloxy) dodecanoic acid (12:0/18:1-OAHFA). Mp 35° C. ¹H NMR (500.13 MHz, CDCl₃, 25° C.) δ 5.38-5.30 (m, 2H), 4.05 (t, 2H), 2.35 (t, 2H), 2.29 (t, 2H), 2.03-1.99 (m, 4H), 1.66-1.58 (m, 6H), 1.33-1.27 (m, 34H) and 0.88 (t, 3H) ppm. ¹³C-NMR (125.68 MHz, CDCl₃, 25° C.): δ 177.0, 174.2, 130.2, 129.9, 64.6, 34.6, 33.6, 32.1, 29.9-29.2, 28.8, 27.4, 27.3, 26.1, 25.2, 24.8, 22.8, and 14.3 ppm.HRMS m/z calculated for [M+Na]⁺ C₃₀H₅₆O₄Na 503.4067, found 503.4091.

12-(palmitoleoyloxy)dodecanoic acid (12:0/16:1-OAHFA). Mp 36.8° C. ¹H NMR (499.82 MHz, CDCl₃, 25° C.): δ 5.39-5.29 (m, 2H), 4.05 (t, 2H), 2.35 (t, 2H), 2.29 (t, 2H), 2.06-1.95 (m, 4H), 1.69-1.54 (m, 6H), 1.39-1.19 (m, 30H) and 0.88 (t, 3H) ppm. ¹³C NMR (125.68 MHz, CDCl₃, 25° C.): δ 179.2, 174.2, 130.1, 129.9, 64.6, 34.6, 34.0, 31.9, 29.7-29.0, 28.8, 27.4, 27.3, 26.1, 25.2, 24.8, 22.8 and 14.2 ppm. HRMS m/z calculated for C₂₈H₅₂O₄Na [M+Na]⁺ 475.3758, found 475.3681.

12-(palmitoyloxy) dodecanoic acid (12:0/16:0-OAHFA). Mp 66.7° C. ¹H NMR (499.82 MHz, CDCl₃, 25° C.): δ 4.05 (t, 2H), 2.35 (t, 2H), 2.29 (t, 2H), 1.68-1.55 (m, 6H), 1.41-1.16 (m, 38H) and 0.88 (t, 3H) ppm. ¹³C NMR (125.68 MHz, CDCl₃, 25° C.): δ 179.2, 174.2, 64.6, 34.6, 34.0, 32.1, 29.8-29.2, 28.8, 26.1, 25.2, 24.8, 22.8 and 14.3 ppm. HRMS m/z calculated for C₂₈H₅₄O₄Na [M+Na]⁺ 477.3914, found 477.3868.

12-(stearoyloxy) dodecanoic acid (12:0/18:0-OAHFA). Mp 71.2° C. ¹H NMR (499.82 MHz, CDCl₃, 25° C.): δ 4.05 (t, 2H), 2.35 (t, 2H), 2.29 (t, 2H), 1.68-1.53 (m, 6H), 1.39-1.17 (m, 42H) and 0.88 (t, 3H) ppm. ¹³C NMR (125.68 MHz, CDCl₃, 25° C.): δ 178.9, 174.2, 64.5, 34.6, 34.0, 32.1, 29.8-29.2, 28.8, 26.1, 25.2, 24.8, 22.8 and 14.3 ppm. HRMS m/z calculated for C₃₀H₅₈O₄Na [M+Na]⁺ 505.4227, found 505.4251.

12-(linoleoyloxy) dodecanoic acid (12:0/18:2-OAHFA). ¹H NMR (499.82 MHz, CDCl₃, 25° C.): δ 5.42-5.28 (m, 4H), 4.05 (t, 2H), 2.77 (m, 2H), 2.35 (t, 2H), 2.29 (t, 2H), 2.08-1.99 (m, 4H), 1.69-1.53 (m, 6H), 1.39-1.16 (m, 28H) and 0.88 (t, 3H) ppm. ¹³C NMR (125.68 MHz, CDCl₃, 25° C.): δ 178.9, 174.2, 130.4, 130.2, 128.2, 128.1, 64.6, 34.6, 33.9, 31.7, 29.8-29.2, 28.8, 27.4, 27.3, 26.1, 26.1, 25.8, 25.2, 24.8, 22.7 and 14.2 ppm. HRMS m/z calculated for C₃₀H₅₄O₄Na [M+Na]⁺ 501.3914, found 501.3931.

15-(oleoyloxy) pentadecanoic acid (15:0/18:1-OAHFA). Mp 50° C. ¹H NMR (500.13 MHz, CDCl₃, 25° C.): δ 5.38-5.30 (m, 2H), 4.06 (t, 2H), 2.35 (t, 2H), 2.29 (t, 2H), 2.03-1.99 (m, 4H), 1.67-1.59 (m, 6H), 1.30-1.26 (m, 40H) and 0.88 (t, 3H) ppm. ¹³C-NMR (125.68 MHz, CDCl₃, 25° C.): δ 176.9, 174.2, 130.2, 129.9, 64.6, 34.5, 33.6, 32.1, 29.9-29.3, 26.1, 25.2, 24.9, 22.8, and 14.3 ppm. HRMS m/z calculated for [M+Na]⁺ C₃₃H₆₂O₄Na 545.4546, found 545.4529.

20-(palmitoleoyloxy) eicosanoic acid (20:0/16:1-OAHFA). Mp 61.0° C. ¹H NMR (499.82 MHz, CDCl₃, 25° C.): δ 5.39-5.30 (m, 2H), 4.05 (t, 2H), 2.35 (t, 2H), 2.29 (t, 2H), 2.05-1.96 (m, 4H), 1.68-1.54 (m, 6H), 1.38-1.20 (m, 46H) and 0.88 (t, 3H) ppm. ¹³C NMR (125.68 MHz, CDCl₃, 25° C.): δ 178.6, 174.2, 130.1, 129.9, 64.6, 34.6, 33.9, 31.9, 29.9-29.1, 28.8, 27.4, 27.3, 26.1, 25.2, 24.9, 22.8 and 14.2 ppm. HRMS m/z calculated for C₃₆H₆₈O₄Na [M+Na]⁺ 587.5010, found 587.5051.

20-(palmitoyloxy) eicosanoic acid (20:0/16:0-OAHFA). Mp 76.8° C. ¹H NMR (499.82 MHz, CDCl₃, 25° C.): δ 4.05 (t, 2H), 2.35 (t, 2H), 2.29 (t, 2H), 1.69-1.51 (m, 6H), 1.49-1.14 (m, 54H) and 0.88 (t, 3H) ppm. ¹³C NMR (125.68 MHz, CDCl₃, 25° C.): δ 177.5, 174.2, 64.6, 34.6, 33.7, 32.1, 29.8-29.2, 28.8, 26.1, 25.2, 24.9, 22.8 and 14.3 ppm. HRMS m/z calculated for C₃₆H₇₀O₄Na [M+Na]⁺ 589.5166, found 589.5145.

20-(linoleoyloxy) eicosanoic acid (20:0/18:2-OAHFA). Mp 53.6° C. ¹H NMR (499.82 MHz, CDCl₃, 25° C.): δ 5.42-5.28 (m, 4H), 4.05 (t, 2H), 2.77 (m, 2H), 2.35 (t, 2H), 2.29 (t, 2H), 2.08-1.99 (m, 4H), 1.67-1.55 (m, 6H), 1.40-1.19 (m, 44H) and 0.89 (t, 3H) ppm. ¹³C NMR (125.68 MHz, CDCl₃, 25° C.): δ 178.6, 174.2, 130.4, 130.2, 128.2, 128.1, 64.6, 34.6, 33.9, 31.7, 29.8-29.2, 28.8, 27.4, 26.1, 25.8, 25.2, 24.9, 22.7 and 14.2 ppm. HRMS calculated for C₃₈H₇₀O₄Na [M+Na]⁺ 613.5166, found 613.5164.

20-(stearoyloxy) eicosanoic acid (20:0/18:0-OAHFA). ¹H NMR (499.82 MHz, CDCl₃, 25° C.): δ 4.05 (t, 2H), 2.35 (t, 2H), 2.29 (t, 2H), 1.68-1.55 (m, 6H), 1.39-1.17 (m, 58H) and 0.88 (t, 3H) ppm. ¹³C NMR (125.68 MHz, CDCl₃, 25° C.): δ 178.9, 174.1, 64.4, 34.4, 33.9, 31.9, 29.8-29.1, 28.7, 26.0, 25.0, 24.7, 22.7 and 14.1 ppm. HRMS calculated for C₃₈H₇₄O₄Na [M+Na]⁺ 617.5479, found 617.5455.

20-(oleoyloxy) eicosanoic acid (20:0/18:1-OAHFA). Mp 62° C. ¹H NMR (500.13 MHz, CDCl₃, 25° C.): δ 5.38-5.30 (m, 2H), 4.06 (t, 2H), 2.35 (t, 2H), 2.29 (t, 2H), 2.03-1.99 (m, 4H), 1.67-1.55 (m, 6H), 1.30-1.25 (m, 50H) and 0.88 (t, 3H) ppm. ¹³C NMR (125.68 MHz; CDCl₃, 25° C.): δ 177.1, 174.2, 130.2, 129.9, 64.6, 34.6, 33.7-27.3, 26.1, 25.2, 24.9, 22.8 and 14.3 ppm. HRMS m/z calculated for [M+K]⁺ C₃₈H₇₂O₄K 631.5068, found 631.5005.

22-(oleoyloxy) docosanoic acid (22:0/18:1-OAHFA). ¹H NMR (500.13 MHz, CDCl₃, 25° C.): δ 5.38-5.30 (m, 2H), 4.05 (t, 2H), 2.35 (t, 2H), 2.29 (t, 2H), 2.03-1.97 (m, 4H), 1.67-1.55 (m, 6H), 1.30-1.25 (m, 54H) and 0.88 (t, 3H) ppm. ¹³C NMR (125.68 MHz; CDCl₃, 25° C.): δ 179.4, 174.2, 130.1, 129.9, 64.6, 34.6, 34.0-29.2, 28.8, 27.4, 27.3, 26.1, 25.2, 24.9, 22.8 and 14.3 ppm. HRMS m/z calculated for C₄₀H₇₆O₄Na [M+Na]⁺ 643.5636, found 643.6390.

### Example 2. Synthesis of (12Z)-20-(oleoyloxy)eicos-12-enoic acid (20:1/18:1-OAHFA)

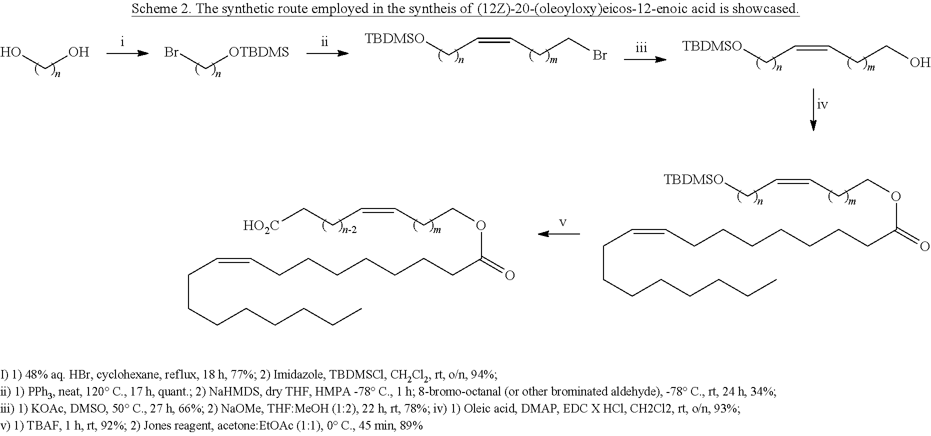

12-bromo-1-dodecanol. To a solution containing 1,12-dodecanediol (2 g, 1 equiv.) in cyclohexane (26 ml) was added HBr (26 ml, 24 eq., 48% sol. In H₂O) and the biphasic system was refluxed for 18 h. The reaction mixture was then cooled to rt and the organic layer was separated. The aqueous phase was extracted with CH₂Cl₂ (5×25 ml). The combined organic phase was washed with sat. aq. NaHCO₃ solution (5×25 ml), brine (50 ml), dried over Na₂SO₄, filtered and concentrated. The crude product was purified by column chromatography (Hexane:EtOAc 7:3) and dried on the vacuum line to give the title compound as a white solid (2 g, 77% yield). ¹H NMR (500.13 MHz, CDCl₃): δ 3.64 (t, 2H), 3.41 (t, 2H), 1.85 (tt, 2H), 1.56 (tt, 2H), 1.42 (tt, 2H) and 1.38-1.22 (m, 14H) ppm.¹³C NMR (125.68 MHz, CDCl₃): δ 63.2, 34.2, 33.0, 29.7-28.9, 28.3 and 25.9 ppm. HRMS m/z calculated for C₁₂H₂₅BrONa [M+Na]⁺ 287.0989, found 287.1001.

12-bromo-1-tert-butyldimethylsilyloxydodecane. To a solution containing 12-bromo-1-dodecanol (0.500 g, 1.2 equiv.) in dry CH₂Cl₂ (3 ml) was added imidazole (0.215 g, 2 equiv.). The reaction mixture was stirred under an argon atmosphere and after complete dissolution, TBDSMCI (0.238 g, 1 eq.) was added. The reaction mixture was stirred at rt for 18 h and then poured onto a cold sat. aq. NaHCO₃ solution (20 ml) and extracted with CH₂Cl₂ (3×20 ml). The combined organic phase was washed with H₂O (50 ml), dried over Na₂SO₄, filtered and concentrated. The crude oil was purified by column chromatography (Hexane:EtOAc 97:3) and dried on the vacuum line to give the title compound as a colorless thick oil (0.564 g, 94% yield). ¹H NMR (500.13 MHz, CDCl₃): δ 3.60 (t, 2H), 3.40 (t, 2H), 1.85 (tt, 2H), 1.50 (tt, 2H), 1.42 (tt, 2H), 1.35-1.22 (m, 14H), 0.89 (s, 9H) and 0.04 (s, 6H) ppm. ¹³C NMR (125.68 MHz, CDCl₃): δ 63.5, 34.1, 33.0, 29.8-28.9, 28.3, 26.1, 26.0, 18.5 and −5.1 ppm. HRMS m/z calculated for C₁₈H₃₉BrOSiNa [M+Na]⁺ 401.1854, found 401.1852.

1-tert-butyldimethylsilyloxydodecane, triphenylphosphonium bromide. A mixture containing 12-bromo-1-tert-butyldimethylsilyloxydodecane (2.0 g, 1 equiv.) and PPh₃ (1.39 g, 1 equiv.) was stirred under an argon atmosphere at 120° C. o/n and cooled to rt. The formation of the triphenylphosphonium bromide salt was confirmed by ³¹P NMR-analysis and the thick resinous product was used in the subsequent Wittig reaction as such. ³¹P NMR (202.4 MHz, CDCl₃): δ 24.4 ppm.

8-bromo-1-octanal. 8-bromo-1-octanol (1.01 g, 1 eq.) was dissolved in CH₂Cl₂ (80 ml) and PCC (1.563 g, 1.5 eq.) was added. The reaction mixture was stirred at rt for 3 h and then Et₂O (80 ml) was added, followed by filtration through celite to remove the remnants of PCC. The flask was washed with Et₂O (2×80 ml) and the combined filtrates were filtered through celite before concentration. H₂O (50 ml) and Et₂O (50 ml) were added to this residue. The light green organic phase was separated and the aqueous phase was extracted with CH₂Cl₂ (2×50 ml). The combined organic phase was washed with H₂O (100 ml), dried over Na₂SO₄, filtered and concentrated to give the title compound as an oil (84% yield). The crude product was used in the subsequent Wittig reaction as such. ¹H NMR (500.13 MHz, CDCl₃): δ 9.74 (t, 1H), 3.38 (t, 2H), 2.41 (dt, 2H), 1.83 (tt, 2H), 1.61 (tt, 2H), 1.42 (tt, 2H) and 1.35-1.29 (m, 4H) ppm.

(12Z)-20-Bromo-1-tert-butyldimethylsilyloxyeicos-12-ene. A solution containing 1-tert-butyldimethylsilyloxydodecane, triphenylphosphonium bromide (3.164 g, 2 equiv.) in dry THF (30 ml) and HMPA (8.9 ml) under an argon atmosphere was cooled to −78° C. After 10 min., NaHMDS (8.2 ml, 0.6 M in toluene, 2 equiv.) was slowly added and the resulting mixture was stirred for 1 h. A solution of freshly prepared 8-bromo-1-octanal (0.536 g, 1.05 eq.) dissolved in dry THF (8 ml) was slowly added at −78° C. and the reaction mixture was allowed to warm to rt over 24 h before it was quenched with aq. phosphate buffer (freshly prepared, pH=7.2; 80 ml). Extraction with Et₂O (3×80 ml) was then performed and the combined organic phase was dried over Na₂SO₄, filtered and concentrated. The crude product was purified by column chromatography (Hexane:Et₃N 100:0.1-Hexane:EtOAc:Et₃N 399:1:0.1@98.7:1.3:0.1) and further dried on the vacuum line to give the title compound as a thick yellowish oil (0.427 g, 34% yield). ¹H NMR (499.82 MHz, CDCl₃): δ 5.35 (dtt, 1H), 5.34 (dtt, 1H), 3.60 (t, 2H), 3.40 (t, 2H), 2.02 (ddt, 2H), 2.01 (ddt, 2H), 1.85 (tt, 2H), 1.50 (tt, 2H), 1.43 (tt, 2H), 1.38-1.22 (m, 22H), 0.89 (s, 9H) and 0.05 (s, 6H) ppm.¹³C NMR (125.68 MHz, CDCl₃): δ 130.3, 129.9, 63.5, 34.2, 33.1, 33.0, 29.9-28.8, 28.3, 27.4-27.3, 26.2, 26.0, 18.6 and −5.1 ppm. HRMS m/z calculated for C₂₆H₅₃BrOSiNa [M+Na]⁺ 511.2949, found 511.2995.

(12Z)-20-acetoxy-1-tert-butyldimethylsilyloxyeicos-12-ene. To a solution containing 2 (0.265 g, 1 equiv.) in DMSO (12 ml) was added KOAc (0.266 g, 5 equiv.) and the suspension was stirred at rt o/n. After 24 h, additional KOAc (0.159 g, 3 equiv.) was added and the temperature was raised to 50° C. After 27 h, the reaction mixture was brought to rt and H₂O (25 ml) was added. Extraction with Et₂O (3×25 ml) was performed and the combined organic phase was washed with brine (30 ml), dried over Na₂SO₄, filtered and concentrated. The crude product was purified by column chromatography (Hexane:EtOAc:Et₃N 98:2:0.1-95.5:0.1) and dried on the vacuum line to give the title compound as a yellowish oil (0.169 g, 66% yield). ¹H NMR (500.13 MHz, CDCl₃): δ 5.35 (dtt, 1H), 5.34 (dtt, 1H), 4.05 (t, 2H), 3.59 (t, 2H), 2.04 (s, 3H), 2.01 (ddt, 2H), 2.00 (ddt, 2H), 1.62 (tt, 2H), 1.50 (tt, 2H), 1.39-1.22 (m, 24H), 0.89 (s, 9H) and 0.04 (s, 6H) ppm. ¹³C NMR (125.68 MHz, CDCl₃): δ 171.4, 130.2, 129.9, 64.8, 63.5, 33.1, 29.9-29.3, 28.8, 27.4-27.3, 26.2, 26.0, 21.2, 18.6 and −5.1 ppm. HRMS m/z calculated for C₂₈H₅₆O₃SiNa [M+Na]⁺ 491.3899, found 491.3879.

(12Z)-20-hydroxy-1-tert-butyldimethylsilyloxyeicos-12-ene. To a solution containing (12Z)-20-acetoxy-1-tert-butyldimethylsilyloxyeicos-12-ene (0.022 g, 1 equiv.) in MeOH (1 ml) and THF (0.5 ml) under argon atmosphere was added NaOMe (0.003 g, 1 eq.) and the resulting mixture was stirred at rt. After 22 h, the reaction was quenched by the addition of aq. HCl (10% sol., v/v; 3 drops) and H₂O (15 ml). The resulting mixture was extracted with Et₂O (3×15 ml) and the combined organic phase was dried over Na₂SO₄, filtered and concentrated. Drying under vacuum gave the title compound as a white solid (0.015 g, 78% yield). ¹H NMR (500.13 MHz, CDCl₃): δ 5.34 (dtt, 1H), 5.34 (dtt, 1H), 3.64 (t, 2H), 3.59 (t, 2H), 2.01 (ddt, 2H), 2.00 (ddt, 2H), 1.57 (tt, 2H), 1.50 (tt, 2H), 1.40-1.22 (m, 24H), 0.89 (s, 9H) and 0.04 (s, 6H) ppm. ¹³C NMR (125.68 MHz, CDCl₃): δ 130.2, 129.9, 63.5, 63.3, 33.0, 32.9, 29.9-29.4, 27.4-27.3, 26.1, 26.0-25.9, 18.6 and −5.1 ppm. HRMS (EI) m/z calculated for C₂₆H₅₄O₂SiNa [M+Na]⁺ 449.3793, found 449.3832.

(12Z)-20-oleoyloxy-1-tert-butyldimethylsilyloxyeicos-12-ene. To a solution of 3 (0.040 g, 1 equiv.) in dry CH₂Cl₂ (2 ml) under argon atmosphere was added DMAP (0.012 g, 1 equiv.) and EDC×HCl (0.046 g, 2.5 eq.) and the resulting mixture was cooled to 0° C. on an ice-bath. Oleic acid (0.032 g dissolved in 0.5 ml dry CH₂Cl₂, 1.2 equiv.) was added and the reaction mixture was stirred at 0° C. for 10 min, and then at rt o/n. The reaction mixture was quenched after 20 h with H₂O (2 ml) and diluted with CH₂Cl₂ (10 ml). The organic phase was separated and the aqueous phase extracted with CH₂Cl₂ (2×10 ml). The combined organic phase was washed with H₂O (2×15 ml), dried over Na₂SO₄, filtered and concentrated. The crude product was purified by column chromatography (Hexane:EtOAc:Et₃N 95:5:0.1) and dried under vacuum to give the title compound as a white solid (0.061 g, 93% yield). ¹H NMR (500.13 MHz, CDCl₃): δ 5.35 (dtt, 1H), 5.35 (dtt, 1H), 5.34 (dtt, 1H), 5.34 (dtt, 1H), 4.05 (t, 2H), 3.60 (t, 2H), 2.29 (t, 2H), 2.06-1.96 (m, 8H), 1.62 (tt, 2H), 1.61 (tt, 2H), 1.50 (tt, 2H,), 1.37-1.23 (m, 44H), 0.89 (s, 9H), 0.88 (t, 3H) and 0.05 (s, 6H) ppm. ¹³C NMR (125.68 MHz, CDCl₃): δ 174.1, 130.2-129.9, 64.5, 63.5, 34.5, 33.0, 32.1, 29.9-29.3, 28.8, 27.4-27.3, 26.1, 26.0, 25.2, 22.8, 18.6, 14.3 and −5.1 ppm. HRMS m/z calculated for C₄₆H₈₃O₃SiNa [M+Na]⁺ 713.6246, found 713.6214.

(12Z)-20-oleoyloxyeicos-12-enol (20:1/18:1-OAHFAI). A solution containing (12Z)-20-oleoyloxy-1-tert-butyldimethylsilyloxyeicos-12-ene (0.031 g, 1 equiv.) in dry THF (0.5 ml) under argon atmosphere was cooled to 0° C. on an ice-bath and TBAF (0.140 ml, 1M in THF; 3 equiv.) was added. After 5 min., the ice-bath was removed and the reaction mixture stirred at rt for 1 h and then quenched with H₂O (2 ml) and extracted with EtOAc (3 ml). The organic phase was washed with H₂O (2 5 ml), the aqueous phase re-extracted with EtOAc (2×5 ml). The combined organic phase was dried over Na₂SO₄, filtered and concentrated. The crude product was purified by column chromatography (Hexane:EtOAc:Et₃N 7:3:0.1) and dried under vacuum to give the title compound a white solid (0.023 g, 92% yield). ¹H NMR (500.13 MHz, CDCl₃): δ 5.35 (dtt, 1H), 5.35 (dtt, 1H), 5.34 (dtt, 1H), 5.34 (dtt, 1H), 4.05 (t, 2H), 3.64 (t, 2H), 2.29 (t, 2H), 2.06-1.96 (m, 8H), 1.62 (tt, 2H), 1.61 (tt, 2H), 1.56 (tt, 2H), 1.37-1.23 (m, 44H) and 0.88 (t, 3H) ppm. ¹³C NMR (125.68 MHz, CDCl₃): δ 174.2, 130.2-129.9, 64.5, 63.2, 34.5, 33.0, 32.1, 29.9-29.3, 28.8, 27.4-27.3, 26.0-25.9, 25.2, 22.8 and 14.3. HRMS m/z calculated for C₃₈H₇₂O₃Na [M+Na]⁺ 599.5381, found 599.5342.

(12Z)-20-oleoyloxyeicos-12-enoic acid (20:1/18:1-OAHFA). A solution containing 4 (0.026 g, 1 equiv.) in acetone (2 ml) and EtOAc (2 ml) was cooled to 0° C. on an ice-bath and Jones reagent (0.050 ml, 2.2 equiv.) was added. The resulting mixture was stirred at 0° C. for 45 min. H₂O (5 ml) was added and the reaction mixture was then extracted with Et₂O (3×15 ml). The combined organic phase was washed with brine (15 ml), dried over Na₂SO₄, filtered and concentrated. The crude product was purified by column chromatography (Hexane:EtOAc:AcOH 7:3:0.1) as eluent and dried on the vacuum line to give the title compound as a white solid (0.024 g, 89% yield). Mp: 29.5-30.7° C. ¹H NMR (500.13 MHz, CDCl₃): δ 5.35 (dtt, 1H), 5.35 (dtt, 1H), 5.34 (dtt, 1H,), 5.34 (dtt, 1H), 4.05 (t, 2H), 2.34 (t, 2H), 2.29 (t, 2H), 2.06-1.96 (m, 8H), 1.63 (tt, 2H), 1.61 (tt, 2H), 1.61 (tt, 2H), 1.37-1.23 (m, 42H) and 0.88 (t, 3H) ppm. ¹³C NMR (125.68 MHz, CDCl₃): δ 179.6, 174.2, 130.1-129.9, 64.6, 34.5, 34.4, 32.0, 29.9-29.3, 28.8, 27.4-27.3, 26.1, 25.2, 24.9, 22.8 and 14.3. HRMS m/z calculated for C₃₈H₇₀O₄Na [M+Na]⁺ 613.5174, found 613.5175.

### Example 3. (21Z)-29-(oleoyloxy) nonacos-21-enoic acid (29:1/18:1-OAHFA)

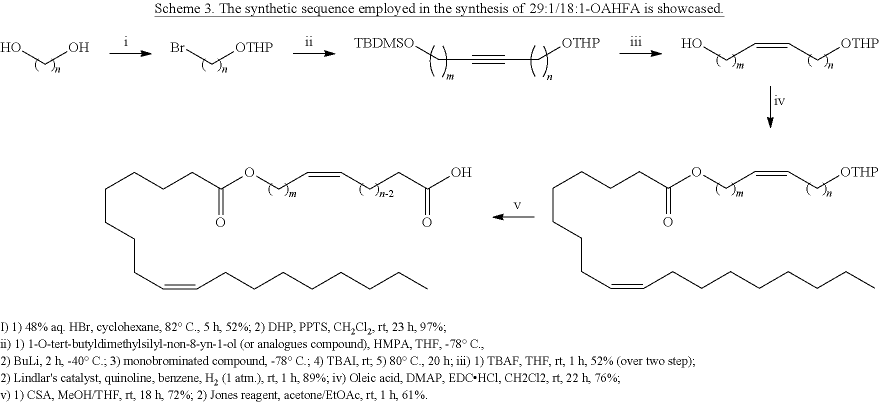

1,20-eicosanediol. A solution containing eicosanedioic acid (0.5101 g, 1 equiv.) in 150 ml of THF was cooled to 0° C. on an ice bath. LAH (0.3419 g, 6.05 equiv.) was added portion wise and the mixture was brought to rt. The reaction mixture was heated to 85° C. and stirred for 18 h. The reaction was cooled down to 0° C. on an ice bath and satd. Aqueous solution containing Rochelle's salt (40 ml) was added. The resulting mixture was stirred for 1 h and filtered through a celite pad. The filtrate was extracted with DCM (8×30 ml). The combined organic layers were dried over Na₂SO₄, filtered and concentrated to give the title compound as a white solid (0.405 g, 87% yield). ¹H NMR (500.13 MHz, CDCl₃, 25° C.): δ 3.64 (t, 4H), 1.56 (tt, 4H) and 1.38-1.15 (m, 32H) ppm. ¹³C NMR (125.68 MHz, CDCl₃, 25° C.): δ 63.2, 32.8, 29.7-29.6, 29.4 and 25.8 ppm. HRMS m/z calculated for C₂₀H₄₂O₂Na [M+Na]⁺ 337.3083, found 337.3152.

20-bromoeicosan-1-ol. A mixture containing 1,20-eicosanediol (0.66 g, 1 equiv.), cyclohexane (24 ml) and HBr (9 ml, 48% in water) was heated at 82° C. for 5 h. The reaction was quenched with H₂O (20 ml) and the layers were separated. The aqueous layer was extracted with CH₂Cl₂ (4×20 ml). The combined organic layers were washed with a satd. Aqueous solution of NaHCO₃ (30 ml) and H₂O (30 ml). The organic phase was separated, dried over Na₂SO₄, filtered and concentrated. The crude product was purified by flash chromatography (Hexane:EtOAc 2:1) and dried on the vacuum line to give the title compound as a white solid (0.42 g, 52% yield). ¹H NMR (499.82 MHz, CDCl₃, 25° C.): δ 3.64 (dt, 2H), 3.40 (t, 2H), 1.85 (tt, 2H), 1.56 (tt, 2H), 1.42 (tt, 2H), and 1.38-1.10 (m, 31H) ppm.¹³C NMR (125.68 MHz, CDCl₃, 23° C.): δ 63.2, 34.2, 33.0, 29.8-28.9, 28.3, and 25.9 ppm. HRMS m/z calculated for C₂₀H₄₁ObrNa [M+Na]⁺ 399.2233, found 399.2185.

20-bromo-1-(2′-tetrahydropyranyloxy) eicosane. To a solution containing 20-bromoeicosan-1-ol (0.23 g, 1 equiv.) in CH₂Cl₂ (20 ml) was added PPTS (0.03 g, 0.13 equiv.) and DHP (0.1 ml, 2.15 equiv.). The resulting mixture was stirred for 23 h at rt. The crude product was after concentration purified by flash chromatography (Hexane:EtOAc 9:1) to give the title compound as a white solid (0.27 g, 97% yield). ¹H NMR (499.82 MHz, CDCl₃, 23° C.): 4.57 (dd, 1H), 3.87 (ddd, 1H), 3.73 (dt, 1H), 3.50 (m, 1H), 3.40 (t, 2H), 3.38 (dt, 1H), 1.85 (tt, 2H), (ddddd, 1H), 1.71 (dddd, 1H), 1.63-1.48 (m, 6H), 1.42 (tt, 2H), and 1.39-1.19 (m, 30H) ppm.

¹³C NMR (125.68 MHz, CDCl₃, 23° C.): δ 99.0, 67.8, 62.5, 34.2, 33.0, 30.9, 29.9-28.9, 28.3, 26.4, 25.7, and 19.9 ppm. HRMS m/z calculated for C₂₅H₄₉ObrNa [M+Na]⁺ 483.2814, found 483.2736.

1-O-tert-butyldimethylsilyl-non-8-yne. A solution containing non-8-yn-ol (0.5 g, 1 equiv.) and imidazole (0.567 g, 2.25 equiv.) in CH₂Cl₂ (20 ml) was cooled to 0° C. on an ice bath before adding TBDMSCI (1.0 g, 1.8 equiv.). The mixture was brought to rt and stirred for 18 h. The reaction was quenched by pouring the reaction mixture into 20 ml of an ice-cold satd. Aqueous solution of NH₄Cl. The aqueous layer was separated and extracted with CH₂Cl₂ (4×30 ml). The crude product was purified by flash chromatography (EtOAc:hexane 9:1) and dried on the vacuum line to give the title compound as a colorless liquid (0.70 g, 78% yield). ¹H NMR (500.13 MHz, CDCl₃, 25° C.): δ 3.58 (t, 2H), 2.18 (dt, 2H), 1.93 (t, 1H), 1.53 (tt, 2H), 1.51 (tt, 2H), 1.40 (m, 2H), 1.35-1.28 (m, 4H), 0.89 (s, 9H) and 0.05 (s, 6H) ppm.¹³C NMR (125.68 MHz, CDCl₃, 25° C.): 84.9, 68.2, 63.4, 33.0, 29.1, 28.9, 28.6, 26.1, 25.8, 18.5 and −5.3 ppm. HRMS m/z calculated for C₁₅H₃₀Osi [M+Na]⁺ 277.1866, found 277.1924.

29-hydroxy-1-(2′-tetrahydropyranyloxy) nonacos-21-yne. The solution of 1-O-tert butyldimethylsilyl-non-8-yn-1-ol (0.236 g, 2.53 equiv.) in THF (3 ml) and HMPA (1 ml) was cooled to −78° C. using an EtOAc/N₂ bath. To this solution, BuLi (0.25 ml, 2.39 equiv., 2.5 M in THF) was added dropwise and the temperature was allowed to rise to −40° C. and maintained for 2 h. The reaction mixture was then cooled back down to −78° C. and 20-bromo-1-(tetrahydro-2H-pyran-2-yloxy)-eicosanol (1 equiv.) in THF (3 ml) was added dropwise. The resulting mixture was brought to rt, TBAI (0.013 g, 1 mol-%) was added and the temperature was raised to 80° C. The reaction was quenched after 20 h by pouring it onto a satd. Aqueous solution of NH₄Cl (20 ml). The aqueous layer was separated and extracted with EtOAc (6×20 ml). The combined organic phase was dried over Na₂SO₄, filtered and concentrated. The crude product was dissolved in THF (10 ml) and the solution was cooled to 0° C. on an ice bath. TBAF (2.5 ml, 6.83 equiv., 1 M in THF) was added and the reaction mixture was brought to rt and stirred for 1 h. The reaction was quenched with H₂O (20 ml) and the aqueous layer was extracted with CH₂Cl₂ (5×20 ml). The combined organic phase was dried over Na₂SO₄, filtered and concentrated. The crude product was purified by flash chromatography (Hexane:EtOAc 9:1-4:1) and the fractions containing product were collected, concentrated and dried on the vacuum line to give the title compound as a white solid (0.099 g, 52% yield). ¹H NMR (499.82 MHz, CDCl₃, 25° C.): δ 4.57 (dd, 1H), 3.87 (ddd, 1H), 3.73 (dt, 1H), 3.64 (dt, 2H), 3.50 (dddd, 1H), 3.38 (dt, 1H), 2.14 (tt, 2H), 2.13 (tt, 2H), 1.83 (m, 1H), 1.71 (dddd, 1H), 1.62-1.43 (m, 12H) and 1.43-1.20 (m, 39H) ppm. ¹³C NMR (125.68 MHz, CDCl₃, 25° C.): δ 99.0, 80.5-80.2, 67.9, 63.3, 62.5, 32.9, 30.9, 29.9-28.9, 26.4, 25.8, 25.7, 19.9 and 18.9 ppm. HRMS m/z calculated for C₃₄H₆₄O₃Na [M+Na]⁺ 543.4753, found 543.4648.

(21Z)-29-hydroxy-1-(2′-tetrahydropyranyloxy) nonacos-21-ene. 29-Hydroxy-1-(2′-tetrahydropyranyloxy) nonacos-21-yne (0.049 g, 1.0 equiv.) was dissolved in dry benzene (15 ml), and, Lindlar's catalyst (0.025 g) and quinoline (0.11 g, 9.0 equiv.) were added. The resulting mixture was placed inside a reactor and the air was replaced by a H₂-atmosphere (1 atm). The reaction mixture was stirred for 1 h at rt. The hydrogen gas was removed and the reaction mixture was filtered through a pad of celite and concentrated. The crude product was purified by flash chromatography (Hexane:EtOAc 4:1) and dried on the vacuum line to give the title compound as a white solid (0.044 g, 89% yield). ¹H NMR (499.82 MHz, CDCl₃, 25° C.): δ 5.33 (dtt, 1H), 5.32 (dtt, 1H), 4.56 (dd, 1H), 3.86 (ddd, 1H), 3.71 (dt, 1H), 3.61 (t, 2H), 3.48 (dddd, 1H), 3.37 (dt, 1H), 2.00 (ddt, 2H), 1.99 (ddt, 2H), 1.82 (ddddd, 1H), 1.70 (dddd, 1H), 1.62-1.45 (m, 8H) and 1.39-1.14 (m, 44H) ppm. ¹³C NMR (125.68 MHz, CDCl₃, 25° C.): δ 130.1, 129.9, 98.9, 67.8, 63.1, 62.4, 32.9, 30.9, 29.9-29.4, 27.3, 26.4, 25.9, 25.6 and 19.8 ppm. HRMS m/z calculated for C₃₄H₆₆O₃Na [M+Na]⁺ 545.4910, found 545.4994.

(21Z)-29-oleoyloxy-1-(2′-tetrahydropyranyloxy) nonacos-21-ene. (21Z)-29-Hydroxy-1-(2′-tetrahydropyranyloxy) nonacos-21-ene (0.044 g, 1 equiv.) was dissolved in CH₂Cl₂ (2 ml) and DMAP (0.012 g, 1.16 equiv.) and EDC·HCl (0.037 g, 2.32 equiv.) was added to the reaction mixture with subsequent cooling on an ice bath. Oleic acid (0.055 g, 2.34 equiv.) in CH₂Cl₂ (2 ml) was added dropwise to the reaction mixture which was thereafter brought to rt and stirred for 22 h. The reaction was quenched by the addition of H₂O (20 ml) and the aqueous layer was isolated and extracted with DCM (7×10 ml). The combined organic phase was dried over Na₂SO₄, filtered and concentrated. The crude product was purified by flash chromatography (Hexane:EtOAc 98:2-1:1) and dried on the vacuum line to give the title compound as a white solid (0.050 g, 76% yield). ¹H NMR (499.82 MHz, CDCl₃, 25° C.): δ 5.40-5.29 (m, 4H, H-21, H-22, H-9″, H-10″), 4.57 (dd, 1H), 4.05 (t, 2H), 3.87 (ddd, 1H), 3.73 (dt, 1H), 3.50 (m, 1H), 3.38 (dt, 1H), 2.29 (t, 2H), 2.07-1.97 (m, 8H), 1.83 (m, 1H), 1.71 (dddd, 1H), 1.65-1.47 (m, 8H), 1.45-1.20 (m, 64H) and 0.88 (t, 3H) ppm. ¹³C NMR (125.68 MHz, CDCl₃, 25° C.): δ 174.1, 130.2-129.9, 99.0, 67.9, 64.5, 62.5, 34.5, 32.0, 31.0, 29.9-29.3, 28.8, 27.4-27.3, 26.4-26.0, 25.7, 25.2, 22.8, 19.9 and 14.3 ppm. HRMS m/z calculated for C₅₂H₉₈O₄Na [M+Na]⁺ 809.7139, found 809.7246.

(21Z)-29-(oleoyloxy) nonacos-21-enol (29:1/18:1-OAHFAI). (8Z)-29-(Tetrahydro-2H-pyran-2-yloxy) nonacos-8-en-1-yl oleate (0.050 g, 1 equiv.) was dissolved in MeOH:THF 3:1 (4 ml) and the resulting mixture was cooled to on an ice bath and CSA (0.003 g, 0.23 equiv.) was added. The reaction mixture was brought to rt and stirred for 18 h. The reaction was quenched by the addition of H₂O (20 ml) and the aqueous layer was extracted with CH₂Cl₂ (4×20 ml). The combined organic phase was dried over Na₂SO₄, filtered and concentrated. The crude product was purified by flash chromatography (Hexane:EtOAc 9:1) and dried on the vacuum line to give the title compound as a white solid (0.032 g, 72% yield). Mp 48.7-49.7° C. ¹H NMR (499.82 MHz, CDCl₃, 25° C.): 5.40-5.29 (m, 4H), 4.05 (t, 2H), 3.64 (dt, 2H), 2.29 (t, 2H), 2.07-1.97 (m, 8H), 1.62 (tt, 2H), 1.62 (tt, 2H), 1.60 (tt, 2H), 1.40-1.21 (m, 62H), 1.19 (t, 1H) and 0.88 (t, 3H) ppm. ¹³C NMR (125.68 MHz, CDCl₃, 25° C.): δ 174.1, 130.2-129.9, 64.5, 63.3, 33.0, 33.7, 32.1, 29.9-29.2, 28.8, 27.4-27.3, 26.1, 25.9, 25.2, 22.8 and 14.3 ppm. HRMS m/z calculated for C₄₈H₉₀O₃Na [M+H]⁺ 703.6968, found 703.7028.

(21Z)-29-(oleoyloxy) nonacos-21-enoic acid (29:1/18:1-OAHFA). To a solution containing (21Z)-29-(oleoyloxy) nonacos-21-en-1-ol in Acetone:EtOAc 1:1 (4 ml) was added Jones reagent (0.05 ml, 2 M) and the resulting mixture was stirred for 1 h. The reaction was quenched with isopropanol (1 ml) and filtered through celite. After careful washing with EtOAc, the combined organic layers were washed with brine (2×30 ml), separated, dried over Na₂SO₄, filtered and concentrated. The crude product was purified by flash chromatography (Hexane:EtOAc 9:1) and dried on the vacuum line to give the title compound as a white solid (0.02 g, 61% yield). Mp 53.0-54.0° C. ¹H NMR (499.82 MHz, CDCl₃, 25° C.): δ 5.40-5.29 (m, 4H), 4.05 (t, 2H), 2.35 (t, 2H), 2.29 (t, 2H), 2.07-1.97 (m, 8H), 1.64 (tt, 2H), 1.63 (tt, 2H), 1.62 (tt, 2H), 1.40-1.21 (m, 60H) and 0.88 (t, 3H) ppm. ¹³C NMR (125.68 MHz, CDCl₃, 25° C.): δ 175.6, 174.2, 130.2-129.9, 64.6, 34.6, 33.7, 32.1, 29.9-29.2, 28.8, 27.4-27.3, 26.1, 25.2, 24.9, 22.8 and 14.3 ppm. HRMS m/z calculated for C₄₇H₈₈O₄Na [M+Na]⁺ 739.6581 found 739.6803.

### Example 4. Synthesis of (Z)-18-(oleyloxy)-18-oxo-octadecanoic acid (R-18:0/18:1-OAHFA) and Similar Compounds

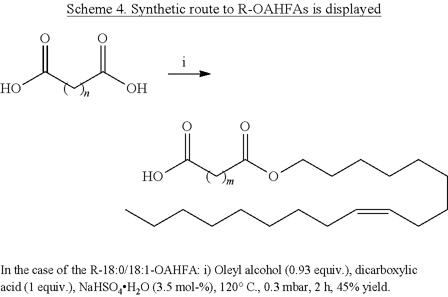

(Z)-18-(oleyloxy)-18-oxo-octadecanoic acid (R-18:0/18:1-OAHFA). 1,18-octadecanedioic acid (1.00 g, 3.2 mmol, 1 equiv.), oleyl alcohol (0.798 g, 3.0 mmol, 0.93 equiv.) and NaHSO₄·H₂O (0.0159 g, 3.5 mol-%) were added to a round bottom flask (100 ml). The stirred mixture was heated to 120° C. on an oil bath under vacuum. After 2 hours, the reaction was brought to r.t., diluted with CHCl₃ (40 ml) and washed with a sat. solution of NaHCO₃ (2×40 ml). The aqueous phase was re-extracted with CHCl₃ (2×40 ml) and the organic layers were combined and washed with Brine (80 ml), dried over Na₂SO₄, filtered and concentrated. The crude product was purified by column chromatography (Hexane:EtOAc:AcOH 4:1:0.01-7:3:0.01), concentrated and dried on the vacuum line to give the title compound as a white solid (0.759 g, 45% yield). Mp: 62.3-63.1° C. ¹H NMR (499.82 MHz, CDCl₃, 25° C.): δ 5.39-5.29 (m, 2H), 4.05 (t, 2H), 2.34 (t, 2H), 2.29 (t, 2H), 2.06-1.97 (m, 4H), 1.67-1.56 (m, 6H), 1.37-1.21 (m, 46H) and 0.88 (t, 3H) ppm. ¹³C NMR (125.68 MHz, CDCl₃, 25° C.): δ 179.5, 174.2, 130.1, 129.9, 64.6, 34.6, 34.1, 32.1, 29.9-29.2, 28.8, 27.4, 27.3, 26.1, 25.2, 24.8, 22.8 and 14.3 ppm. HRMS (EI) m/z calculated for C₃₆H₆₈O₄Na [M+Na]⁺ 587.5015, found 587.4814.

The following compounds were also prepared following analogous synthetic sequences. (Z)-15-(oleyloxy)-15-oxo-pentadecanoic acid (R-15:0/18:1-OAHFA). Mp: 45.5-46.6° C. ¹H NMR (499.82 MHz, CDCl₃, 25° C.): δ 5.39-5.30 (m, 2H), 4.05 (t, 2H), 2.34 (t, 2H), 2.29 (t, 2H), 2.04-1.98 (m, 4H), 1.67-1.57 (m, 6H), 1.37-1.20 (m, 40H) and 0.88 (t, 3H) ppm. ¹³C NMR (125.68 MHz; CDCl₃): δ 179.6, 174.2, 130.1, 129.9, 64.6, 34.6, 34.1, 32.1, 29.9-29.2, 28.8, 27.4, 27.3, 26.1, 25.2, 24.8, 22.8 and 14.3 ppm. HRMS: m/z calculated for C₃₃H₆₂O₄Na [M+Na]⁺ 545.3648, found 545.4530.

(Z)-20-(oleyloxy)-20-oxo-eicosanoic acid (R-20:0/18:1-OAHFA). ¹H NMR (499.82 MHz, CDCl₃, 25° C.): δ 5.39-5.30 (m, 2H), 4.06 (t, 2H), 2.35 (t, 2H), 2.29 (t, 2H), 2.04-1.98 (m, 4H), 1.67-1.57 (m, 6H), 1.37-1.20 (m, 50H) and 0.88 (t, 3H) ppm.

### Further OAHFAS

In addition, the following compounds are prepared by analogous methods to those described above.

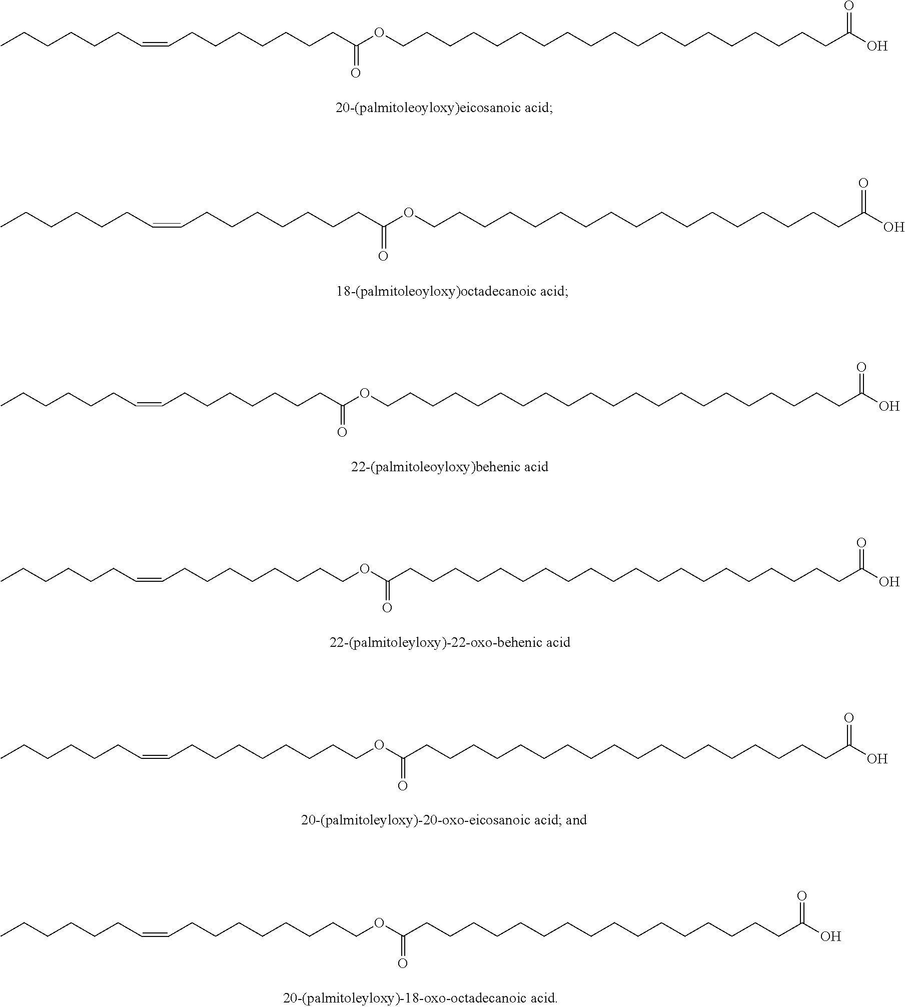 [SMILES: CCCCCC/C=C\CCCCCCCC(=O)OCCCCCCCCCCCCCCCCCCCC(=O)O ; CCCCCC/C=C\CCCCCCCC(=O)OCCCCCCCCCCCCCCCCCC(=O)O ; CCCCCC/C=C\CCCCCCCC(=O)OCCCCCCCCCCCCCCCCCCCCCC(=O)O ; CCCCCC/C=C\CCCCCCCCOC(=O)CCCCCCCCCCCCCCCCCCCCC(=O)O ; CCCCCC/C=C\CCCCCCCCOC(=O)CCCCCCCCCCCCCCCCCCC(=O)O ; CCCCCC/C=C\CCCCCCCCOC(=O)CCCCCCCCCCCCCCCCC(=O)O]

### Synthesis of Wax Esters

A number of commercially available wax esters, i.e. arachidyl laurate, arachidyl oleate, behenyl stearate, behenyl linoleate, behenyl oleate, behenyl behenate, were employed as part of the WE:OAHFA mixtures screened. In addition, non-commercial WEs were synthesized. Here the synthetic routes will be exemplified by the synthesis of hexacosanyl oleate and 24-methylpentacosanyl oleate.

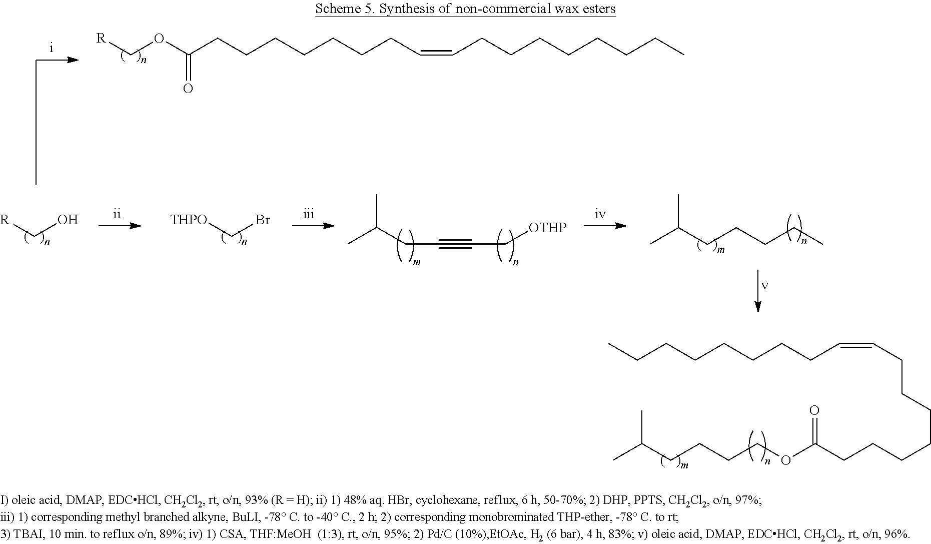

### Example 5. Synthesis of Hexacosyl Oleate

Hexacosyl oleate. To a solution of commercially available 1-hexacosanol (0.03 g, 1 equiv.) in dry CH₂Cl₂ (2.5 ml) and pyridine (1 ml) under argon atmosphere was added DMAP (0.01 g, 1 equiv.) and EDC×HCl (0.0380 g, 2.5 equiv.). Oleic acid (0.027 g in 0.5 ml dry CH₂Cl₂; 1.2 equiv.) was added dropwise. The resulting mixture was stirred o/n at rt. The reaction mixture was quenched after 18.5 h with H₂O (5 ml) and diluted with CH₂Cl₂ (10 ml). The organic phase was separated and the aqueous layer extracted with CH₂Cl₂ (3×10 ml). The combined organic layers were washed with H₂O (2×20 ml), dried over Na₂SO₄, filtered and concentrated. The crude product was purified by column chromatography (Hexane:EtOAc 95:5) and dried on the vacuum line to give the title compound as a white waxy solid (0.047 g, 93% yield). Mp 47.0-49.0° C. ¹H NMR (500.13 MHz, CDCl₃, 25° C.): δ 5.35 (dtt, 1H), 5.33 (dtt, 1H), 4.05 (t, 2H), 2.29 (t, 2H), 2.01 (ddt, 2H), 2.00 (ddt, 2H), 1.62 (tt, 2H), 1.60 (tt, 2H), 1.38-1.19 (m, 66H), 0.88 (t, 3H) and 0.88 (t, 3H) ppm. ¹³C NMR (125.68 MHz, CDCl₃): δ ¹³C NMR (125.68 MHz, CDCl₃, 25° C.): δ 174.1, 130.1, 129.9, 64.5, 39.2, 34.6, 32.1-32.0, 29.9-29.7, 29.5-29.4, 29.3-29.2, 28.8, 27.4-27.3, 26.1, 25.2, 22.8 and 14.3 ppm. HRMS m/z calculated for C₄₄H₈₆O₂Na [M+Na]⁺ 669.6528, found 669.6693.

### Example 6. Synthesis of 24-methylpentacosyl oleate (Iso-WE)

1-(2-tetrahydropyranyloxy)-24-methylpentacos-20-yne. A solution containing 4-methyl-1-pentyne (0.65 ml, 5 equiv.) in dry THF (4 ml) and HMPA (1.5 ml) under an argon atmosphere was cooled to −78° C. n-BuLi (2.2 ml, 2.5 M in hexane, 5 equiv.) was slowly added and the resulting mixture was stirred at −40° C. for 2 h. The temperature was then again lowered to −78° C. and a solution of 20-bromo-1-(2-tetrahydropyranyloxy) eicosane (0.500 g, 1 equiv.) dissolved in dry THF (5 ml) was slowly added and the reaction mixture was allowed to warm to rt. TBAI (0.040 g, 0.1 eq.) was added and the reaction mixture was stirred 10 min. at rt. And then refluxed at 80° C. o/n. The reaction mixture was then quenched with a satd. Solution of NH₄Cl (20 ml) and extracted with Et₂O (5×20 ml). The combined organic phase was washed with H₂O (2×20 ml), dried over Na₂SO₄, filtered and concentrated under reduced pressure. The crude product was purified by column chromatography (Hexane:EtOAc 98:2-90:10) and dried on the vacuum line to give the title compound as a yellowish solid (0.445 g, 89% yield). ¹H NMR (500.13 MHz, CDCl₃): δ 4.57 (t, 1H), 3.92-3.83 (m, 1H), 3.77-3.68 (m, 1H), 3.55-3.46 (m, 1H), 3.42-3.33 (m, 1H), 2.19-2.11 (m, 2H), 2.07-2.01 (m, 2H), 1.88-1.67 (m, 3H), 1.64-1.43 (m, 8H), 1.42-1.33 (m, 4H), 1.32-1.17 (m, 28H), 0.96 (d, 3H) and 0.96 (d, 3H) ppm. ¹³C NMR (125.68 MHz, CDCl₃): δ 98.9, 81.3, 79.2, 67.9, 62.5, 30.9, 29.9-29.0, 28.5, 28.2, 26.4, 25.7, 22.1, 19.9 and 18.9 ppm. HRMS m/z calculated for C₃₁H₅₈O₂Na [M+Na]⁺ 485.4337, found 485.4325.

To a solution 1-hydroxy-24-methylpentacos-20-yne. of 1-(2-tetrahydropyranyloxy)-24-methylpentacos-20-yne (0.153 g, 1 equiv.) in dry THF/MeOH (6 ml, 1:3 ratio) under argon atmosphere was added CSA (0.008 g, 0.1 eq.) and the resulting reaction mixture was stirred at rt. o/n and then concentrated under reduced pressure. The crude product was purified by column chromatography (Hexane:EtOAc 100:0-4:1) and dried on the vacuum line to give the title compound as a white solid (0.118 g, 95% yield). ¹H NMR (500.13 MHz, CDCl₃): δ 3.64 (t, 2H), 2.17-2.12 (m, 2H), 2.06-2.00 (m, 2H), 1.81-1.70 (m, 1H), 1.57 (tt, 2H), 1.48 (tt, 2H), 1.41-1.20 (m, 32H), 0.96 (d, 3H) and 0.96 (d, 3H) ppm. ¹³C NMR (125.68 MHz, CDCl₃): δ 81.3, 79.2, 63.3, 32.9, 29.8-29.0, 28.5, 28.2, 25.9, 22.1 and 18.9 ppm. HRMS m/z calculated for C₂₆H₅₀Ona [M+Na]⁺ 401.3762, found 401.3760.

1-hydroxy-24-methylpentacosane. To a solution of 1-hydroxy-24-methylpentacos-20-yne (0.110 g, 1.0 equiv.) in dry EtOAc (15 ml) Pd/C (10% Pd, 0.220 g, 2 mass equiv.) was added. The reaction mixture was stirred in an autoclave under H₂-pressure (6 bar) for 4 h and filtered through celite. The celite was washed with EtOAc (20 ml) and the filtrate concentrated under reduced pressure. The crude product was purified (Hexane:EtOAc 4:1) and dried on the vacuum line to give the title compound as a white solid (0.093 g, 84% yield). ¹H NMR (500.13 MHz, CDCl₃): δ 3.64 (t, 2H), 1.56 (tt, 2H), 1.53-1.47 (m, 1H), 1.39-1.18 (m, 40H), 1.15 (q, 2H), 0.86 (d, 3H) and 0.86 (d, 3H) ppm. ¹³C NMR (125.68 MHz, CDCl₃): δ 63.3, 39.2, 32.9, 30.1, 29.9-29.6, 28.1, 27.6, 25.9 and 22.8 ppm. HRMS m/z calculated for C₂₆H₅₄Ona [M+Na]⁺ 405.4075, found 405.4028.

24-methylpentacosyl oleate (Iso-WE).

To a solution of 1-hydroxy-24-methylpentacosane (0.035 g, 1 equiv.) in dry CH₂Cl₂ (2.5 ml) and dry pyridine (1 ml) under argon atmosphere was added DMAP (0.012 g, 1 equiv.) and EDC×HCl (0.044 g, 2.5 equiv.). Oleic acid (0.031 g in 0.5 ml dry CH₂Cl₂; 1.2 equiv.) was added dropwise. The resulting mixture at rt. o/n. The reaction mixture was quenched after 18 h with H₂O (10 ml) and diluted with CH₂Cl₂ (10 ml). The organic phase was separated and the aqueous layer extracted with CH₂Cl₂ (4×10 ml). The combined organic layers were washed with H₂O (2×15 ml), dried over Na₂SO₄, filtered and concentrated. The crude product was purified by column chromatography (Hexane:EtOAc 95:5) and dried on the vacuum line to give the title compound as a white waxy solid (0.057 g, 96% yield). Mp 36.0-37.5° C. ¹H NMR (500.13 MHz, CDCl₃): δ 5.36 (dtt, 1H), 5.34 (dtt, 1H), 4.05 (t, 2H), 2.29 (t, 2H), 2.01 (ddt, 2H), 2.00 (ddt, 2H), 1.62 (tt, 2H), 1.60 (tt, 2H), 1.56-1.47 (m, 1H), 1.38-1.19 (m, 60H), 1.15 (q, 2H), 0.88 (t, 3H, overlapping), 0.86 (d, 3H, overlapping) and 0.86 (d, 3H, overlapping) ppm. ¹³C NMR (125.68 MHz, CDCl₃): δ 174.1, 130.1, 129.9, 64.6, 39.2, 34.6, 32.1, 30.1, 29.9-29.3, 28.8, 28.1, 27.6, 27.4-27.3, 26.1, 25.2, 22.8 and 14.3 ppm. HRMS m/z calculated for C₄₄H₈₇O₂ [M+H]⁺ 647.6708, found 647.6725.

### Preparation and Characterization of Formulations

In order to reach optimally functioning formulations, i.e. formulations which are capable of delivering the active components in functional form (retained spreading capabilities and evaporation resistant properties) to the ocular surface, a number of different approaches were initially screened. These included oil-in-water emulsions, oil solutions and liposomal formulations. In this section, examples are given of each formulation type and focus is then placed on the methods and protocols used to characterize the structural integrity and biophysical/biological profiles of the liposomal formulations which were found to be the most promising ones. In addition to displaying good functional properties, the formulations must display adequate pharmaceutical properties (e.g., pH, osmolality) in order to allow topical administration to the ocular surface.

### Example 7. Preparation of Oil-In-Water Emulsions

Active components (1%), 0.8% w/v Miglyol 812, 2% w/v Tween 20, 0.5% w/v Kolliphor® EL, 0.7% w/v Span 80, 2.2% w/v glycerin, and 98.2% w/v ultrapure water were mixing at 76° C. and cooled to rt to produce these formulations.

### Example 8. Preparation of Oil Solutions

Active components were mixed with castor oil in concentrations ranging from 1-20% to produce clear oil solutions.

### Example 9. Preparation of the Liposomal Formulations

Liposomal formulations were prepared utilizing one or several of the following six different phosphatidylcholines (PCs); 1,2-ditetradecanoyl-sn-glycero-3-phosphocholine (DMPC), 1,2-dipentadecanoyl-sn-glycero-3-phosphocholine (15:0 PC), 1,2-dipalmitoyl-sn-glycero-3-phosphocholine (DPPC), 1,2-distearoyl-sn-glycero-3-phosphocholine (DSPC), 1,2-diarachidoyl-sn-glycero-3-phosphocholine (DAPC), and 1,2-dioleoyl-sn-glycero-3-phosphocholine (DOPC) (all purchased from Avanti Polar Lipids Inc., Alabaster, AL, USA). The liposomal formulations were prepared by the thin film hydration method.

Briefly, PCs and evaporation-resistant active compounds were dissolved in chloroform and mixed in various ratios (see summary in Table 1). The compositions were heated in a water bath above their phase transition temperatures (60-70° C.) and chloroform was evaporated under reduced pressure using a rotavapor (Rotavapor R-11; Büchi Labortechnik AG, Flawil, Switzerland). The resulting thin lipid layer was hydrated with Milli-Q® ultrapure water (Millipore, Bedford, MA, USA), 0.9% sodium chloride or TBS (50 mM TRIS-buffered saline; pH 7.4, ThermoFisher Scientific) in the water bath. The sample was sonicated in an ultrasonic bath for 10-20 min. (at 60-70° C., 35 kHz, SONOREX SUPER RK 102 H, BANDELIN electronic Gmbh & Co. KG, Berlin, Germany), and thereafter with a probe sonicator for 2-5 min. (20-25%/200 μm amplitude, SONICS Vibracell VCX750 Ultrasonic Processor with ½″ probe and 2 mm tapered microtip, Sonics & Materials, Inc., Newtown, CT, USA) to reach a more desirable particle size for the liposomes.

TABLE 1
Phosphatidylcholines (PCs) and active components
used in liposomal formulations.

| | | PC:active | |
|---|---|---|---|
| | | compound- | Total lipid |
| | | ratios (Mass | concentration |
| PCs/PC mixtures | Active components | ratio) | (% W/V) |
| 14:0 PC (DMPC) | BO:20-OAHFA | 10:1, 7.5:1, 6:1, | 3%-20% |
| | | 5:1, 4:1, 3:1 | |
| | BO:18-OAHFA | 4:1 | 5% |
| | 18-OAHFA | 8:1, 4:1, 2:1 | 3%-5% |
| | 1-Octadecanol:18-OAHFA | 4:1 | 5% |
| 15:0 PC | BO:20-OAHFA | 10:1, 5:1, 2.5:1 | 3.3-4.2% |
| 16:0 PC (DPPC) | BO:20-OAHFA | 10:1, 2:3 | 3.3-5% |
| 18:0 PC (DSPC) | BO:20-OAHFA | 3:1, 2:1, 1:1 | 1.5-16% |
| | BO:18-OAHFA | 3:1, 2:1, 1:1 | 1.5-16% |
| | 18-OAHFA | 4:1, 3:1, 2:1, 1:1 | 1.5-16% |
| | 1-Octadecanol:18-OAHFA | 3:1, 2:1 | 1.5-3% |
| | BO:R-18-OAHFA | 2:1 | 1.5-3% |
| | 1-Octadecanol:R-18- | 2:1 | 1.5-3% |
| | OAHFA | | |
| | R-18-OAHFA | 4:1, 2:1 | 1.5-3% |
| 20:0 PC (DAPC) | BO:20-OAHFA | 6:1, 5:1, 4:1, | 1.5-8% |
| | | 3.5:1, 3:1, 2:1, | |
| | | 1:1 | |
| | BO:18-OAHFA | 6:1, 5:1, 4:1, | 1.5-8% |
| | | 3.5:1 3:1, 2:1, 1:1 | |
| | 18-OAHFA | 6:1, 5:1, 4:1, | 0.75-8% |
| | | 3.5:1 3:1, 2:1, | |
| | | 1:1, 1:2 | |
| | 1-Octadecanol:18-OAHFA | 2:1 | 1.5%, 3% |
| | BO:R-18-OAHFA | 2:1 | 1.5%, 3% |
| | 1-Octadecanol:R-18- | 2:1 | 1.5%, 3% |
| | OAHFA | | |
| | R-18-OAHFA | 4:1, 2:1 | 1.5%, 3% |
| | 1-Octadecanol | 2:1 | 1.5% 3% |
| | 1-Octadecanol:BO | 2:1 | 1.5%, 3% |
| 20:0 PC:18:1 (Δ9- | 18-OAHFA | 1:1 | 1% |
| Cis) PC | | | |
| (DAPC:COPC) | | | |
| 18:1 (Δ9-Cis) PC | 18-OAHFA | 1:1 | 1% |
| (DOPC) | | | |

In general, the liposomes successfully incorporated the evaporation-resistant active ingredients, but for mass ratios below 1:2 (active ingredients to phospholipids), incorporation was sometimes incomplete (as indicated by additional peaks appearing in the DSC thermograms of the liposomes—discussed further below).

The viscosity of the compositions also varied depending on the specific ingredients (and particularly the phospholipid). For example,
when DMPC was used as the phospholipid, there were issues with foaming and viscosity at total lipid contents (i.e, the total lipid and phospholipid components) of from 21% w/v, but contents up to 15-17% w/v were acceptable;
when DSPC was used as the phospholipid, there were issues with foaming and viscosity at total lipid contents of 14% w/v, but contents up to 8% w/v were acceptable.
When DAPC was used as the phospholipid, there were issues with foaming and viscosity at total lipid contents of 5% w/v, but contents up to 3% w/v were acceptable.

### Example 10—Biophysical Characterization Studies of Active Components and Formulations—General Procedures

Biophysical characterization studies were used to profile the properties of the formulations. In more detail, they were used to verify that active components were delivered to the aqueous interface in functional form and that the film formed displayed adaptive features over compression-expansion cycles. A fully functional formulation was one that displayed good spreading and respreading capabilities over compression-expansion cycles and formed an evaporation resistant film on the aqueous surface. Such a formulation is uniquely suited to treating the tear film instability defect associated with many ocular surface conditions. In addition, the biophysical characterization studies showcase the physical mechanism of action.

The lipid components and tailored mixtures dissolved in chloroform, or alternatively, the formulations undergoing characterization studies were spread onto the air-buffer interface of a Langmuir trough, either a Mini trough (KSV, Helsinki, Finland) or a Large trough (KSV NIMA, Biolin Scientific, Espoo, Finland), which was filled with PBS buffer. The samples were allowed to equilibrate for 3 min before starting the measurements. The surface pressure was measured using a Wilhelmy plate, the surface potential was measured using a KSV NIMA Surface Potential Sensor (Espoo, Finland) when relevant and Brewster angle microscopy (BAM) images were captured using a KSV NIMA microBAM (Espoo, Finland) instrument. The films were compressed at a constant rate of 5 or 10 mm/min and the subphase temperature was maintained at 35±1° C. The measurements were performed in an acrylic box under an ozone-free atmosphere to prevent undesired oxidation of the lipids. An ozone-free atmosphere was generated by passing dry air through ODS-3P ozone destruct unit (Ozone solutions, Hull, Iowa) and into the enclosure at a rate of 76 l/min.

The adaptive features of the formulations were uncovered through studying the spreading capabilities of the lipid films over compression-expansion cycles using the Langmuir setup. In more detail, the volume employed of each formulation was the volume required to observe a response in the surface pressure isotherm over one compression-expansion cycle. Altogether, the films were subjected to twenty compression-expansion cycles employing a 50 mm/min. compression/expansion rate and the changes in the surface pressure isotherms were monitored as described above. Evaporation resistance was determined by a modified Langmuir and Schaefer method (Langmuir et al, 1943, J Franklin Inst, 235, 119-162). The measurements were conducted by compressing the film to a specific mean molecular area, ranging between 2-40 Å²/molecule (for individual compounds), or a specified area (for formulations) and placing a tailored desiccant cartridge, with a water-permeable membrane, approximately 2 mm above the aqueous surface. The commercial silica gel containing desiccant cartridges (SP Industries, Warminster, PA) were modified by replacing the membrane with a Millipore Immobilon-P PVDF membrane (450 nm pore size, Bedford, MA). The desiccant cartridge was fixed in position for 5 minutes and the mass of the absorbed water was determined by gravimetric techniques. In order to take into account the water absorbed from the air inside the enclosure, a second background measurement was conducted in parallel inside the enclosed acrylic box. From the measurements, the evaporation resistance was calculated with the equation (1).

$$r = {A}_{desicant} \cdot t \cdot ( w - {w}_{0} ) \cdot ( \frac{1}{m ( l )} - \frac{1}{{m}_{PBS} ( 0 )} ) - \frac{Δ {m}_{tot} \cdot {t}_{i}}{D \cdot ρ \cdot {t}_{tot} \cdot {A}_{Trough}} \qquad ( 1 )$$

The terms used in the equation are as follows: A_{desicant}=area of the desiccant cartridge;
t=measuring time; (w-w₀)=water concentration difference between the aqueous surface and the surface of the desiccant cartridge; m(I)=water amount absorbed in the presence of the lipid film; m_{PBS}(0)=water amount absorbed in the absence of the lipid film; Δmₜₒₜ=mass of evaporated water during the measurement; tᵢ=time point D=diffusion coefficient; ρ=water density; A_{Trough}=area of the Langmuir Trough.

The evaporation reduction was determined as the total reduction of water absorbed by a desiccant cartridge when a lipid film was covering the subphase versus the situation when the lipid film was absent. Equation (2) was utilized in the calculations.

$$reduction ( \% ) = \frac{{m}_{PBS} ( 0 ) - m ( l )}{{m}_{PBS} ( 0 )} \cdot 100 \% \qquad ( 2 )$$

### Formulation Development, Characterization and Assessment

In order to effectively slow/reduce evaporation from the ocular surface, the TFLL fulfils two criteria: 1) the lipids need to spread rapidly and cover the entire aqueous tear film surface as the eye is opened, and, 2) the film formed by the lipids needs to have a condensed structure that prevents or retards the passage of water molecules through it. Accordingly, the same two requirements need are desirable for lipid compositions developed for reducing or preventing the evaporation of water if the approach is intended to provide an answer to the crucial tear film instability defect.

The development of optimized active evaporation-resistant lipids and functional compositions comprising these lipids, is described in the following examples.

### Example 11—Selection of Active Components

Three functional active component solutions for use in the compositions described herein have been identified:OAHFAS, OAHFA-WE combinations and long-chain fatty alcohols.

### OAHFAS

### Phase Behaviour

It has previously been reported that the evaporation resistance of OAHFA species is influenced by their chain lengths, degree of saturation and melting points (Bland et al. Langmuir 2019, 35, 3545-3552; Viitaja et al. J. Org. Chem. 2021, 86, 4965-4976). The evaporation resistance of these species is believed to be due to the properties of the lipid films formed at the aqueous interface.

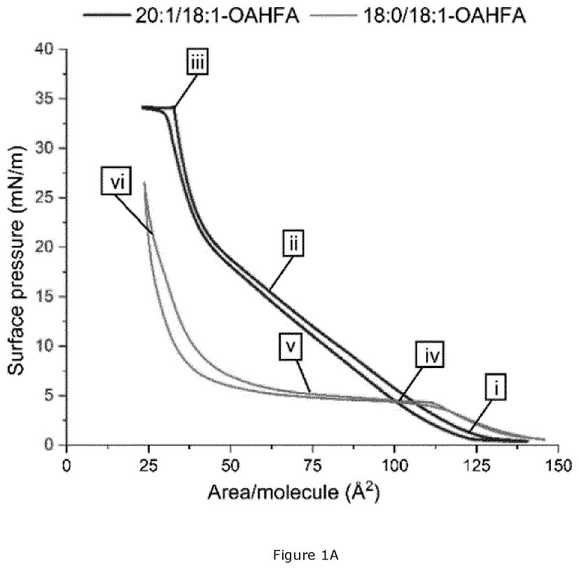

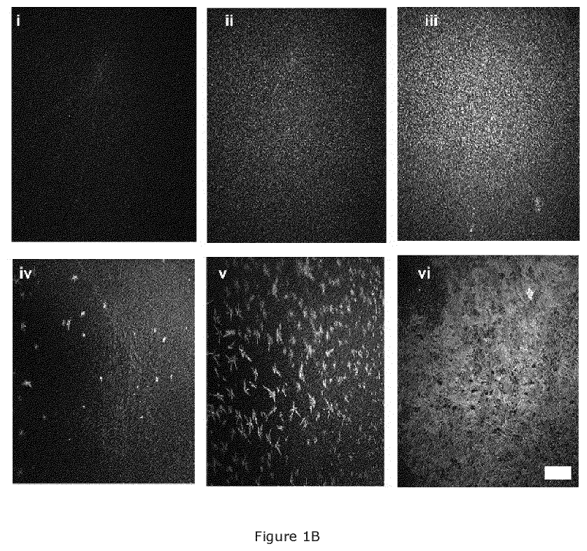

Details on our experimental setup for studying film properties are described in Example 10. In order for a lipid film to display evaporation resistant properties, it needs to form a tightly packed uniform homogenous film without significant structural defects, such as a solid monolayer. It has previously been reported that such a monolayer can form upon phase transition from a liquid to a solid phase (Bland et al. Langmuir 2019, 35, 3545-3552), and that OAHFA species which do not undergo such a phase transition but exist in the liquid phase even at physiological surface pressures do not display evaporation resistant properties (Viitaja et al. J. Org. Chem. 2021, 86, 4965-4976). In FIG. 1, these factors are highlighted through a comparison of the phase behavior of 20:1/18:1-OAHFA and 18:0/18:1-OAHFA as a function of surface pressure and mean molecular area/molecule. When comparing the properties of these two OAHFA species, there are some similarities and differences that need to be noted. First, both OAHFAS are surface active lipids—this is indicated by the surface pressure lift-off occurring at a large mean molecular area/molecule. This behavior has commonly been interpreted as an efficient spreading ability indicating that these species would be capable of spreading tear fluid at the ocular surface-simultaneously fulfilling criteria 1) listed at the beginning of the formulation development, characterization and assessment section page 55.

However, the surface pressure isotherms as well as the accompanied BAM images show distinct trends between these two species. The 20:1/18:1-OAHFA does not undergo a phase transition and exists in the liquid phase throughout the surface pressure range studied. The 18:0/18:1-OAHFA on the other hand, does undergo a liquid to solid phase transition under the studied surface pressure range which is clearly seen in the BAM images. Of these two species, only the films formed by the 18:0/18:1-OAHFA displays evaporation resistance and fulfills criteria 2) listed in the beginning of the results section. Thus, our screening platform enables the rapid identification of promising active components solutions from the OAHFA-category. In fact, the other OAHFA species screened were found to follow similar trends (formation of a densely packed solid monolayer led to an improved evaporation resistance).

### Evaporation Resistance

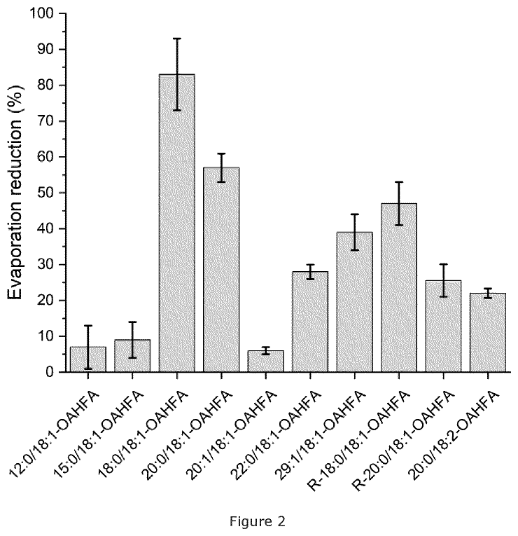

The ability of selected OAHFA films to reduce the evaporation from the aqueous layer was determined. The experimental setup employed in these studies is given in Example 10 and the results are displayed in FIG. 2.

As seen in FIG. 2, the highest evaporation resistance was displayed by the 18:0/18:1-OAHFA film. Films generated from OAHFA-species with similar chain lengths and degrees of saturation (20:0/18:1-OAHFA, 22:0/18:1-OAHFA) also displayed a reasonable degree of evaporation resistance, as did the 29:1/18:1-OAHFA which resembles the naturally occurring OAHFAs, and the R-18:0/18:1-OAHFA and R-20:0/18:1-OAHFA, which contain reversed ester linkages. However, shorter chain lengths (15:0/18:1-OAHFA, 12:0/18:1-OAHFA) displayed significantly weaker evaporation-resistant properties, as did the 20:1/18:1-OAHFA. Of the species tested, the 18:0/18:1-OAHFA species has the most promising biophysical properties for development of functional formulations.

### Wax Ester and OAHFA Combinations

The inventors have recently found that carefully composed mixtures of WEs and OAHFAs may significantly enhance the evaporation resistance of the films formed over either species alone (Viitaja et al. Nano Lett. 2021, 18, 7676-7683). These findings were uncovered while screening the properties of 20:0/18:1-OAHFA, BO and mixtures thereof in ratios varying between 3:1-1:9.

In the course of these studies, it was found that great care needs to be placed on the selection of suitable WEs and OAHFAs in order to provide a combination with improved evaporation resistance compared to the individual species alone. In addition, the WE:OAHFA-ratio greatly influences the biophysical properties on the mixtures and equal/increased amounts of the WE are required to increase the evaporation resistance efficacy. In order to select appropriate WE:OAHFA mixtures for the composition development, a number of mixtures were assessed according to the same principles as detailed above for the case of OAHFAs. In other words, the spreading capabilities of the mixtures, their phase behavior and their ability to reduce water evaporation were determined.

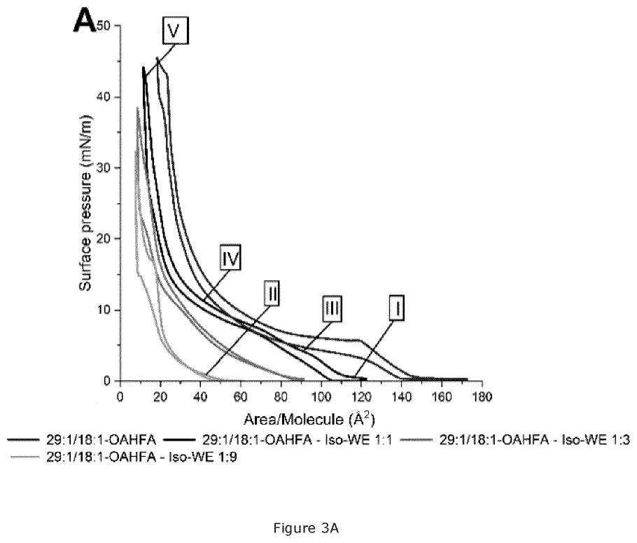

The spreading capabilities and phase behavior of the OAHFA:WE mixtures, will here be exemplified by going through the combination featuring natural-like compounds i.e. 29:1/:18:1-OAHFA and an iso-branched WE. The surface pressure and potential isotherms for OAHFA:WE-mixtures with molar ratios in the range of 1:1-1:9 (OAHFA:WE) are displayed along with selected BAM-images in FIG. 3A-C.

From the surface pressure and potential isotherms it is clear that the behavior of the mixture moves closer to the behavior of the pure 29:1/18:1-OAHFA when the amount of this species is increased. The 1:1 mixture retains good surface active properties as indicated by the large surface pressure lift-off area. This mixture fulfills criteria 1) listed in the beginning of the formulation development section. When the ratio of the wax components is increased, the surface-active properties of the mixture is decreased—a logical outcome considering the properties of wax esters on their own. Therefore, mixtures with increased WE:OAHFA-ratios are not as promising when viewed from the requirements of criteria 1). This is also reflected in the film structure as viewed from the BAM-images. In the 1:1-mixture, a liquid monolayer is initially formed which undergoes a liquid-to-solid phase transition upon an increase in the surface pressure. At physiological surface pressures, a solid condensed monolayer is observed thus adhering to the principles outlined for reaching criteria 2). The OAHFA:WE 1:9-mixture on the other hand does not form a liquid monolayer-instead, formation of solid domains with holes between their boundaries can be observed, this is not ideal for adhering to the requirements for criteria 2). When the results from the OAHFA:WE 1:3-mixture are analyzed, the picture that emerges is that the properties of films formed by different WE:OAHFA-ratios can be estimated based on this series.

Altogether, the 1:1 OAHFA:WE-ratio was found to be the most promising when accounting for selection criteria 1) and 2) and the evaporation resistant properties of several WE:OAHFA-combinations were screened.

### Evaporation Resistance

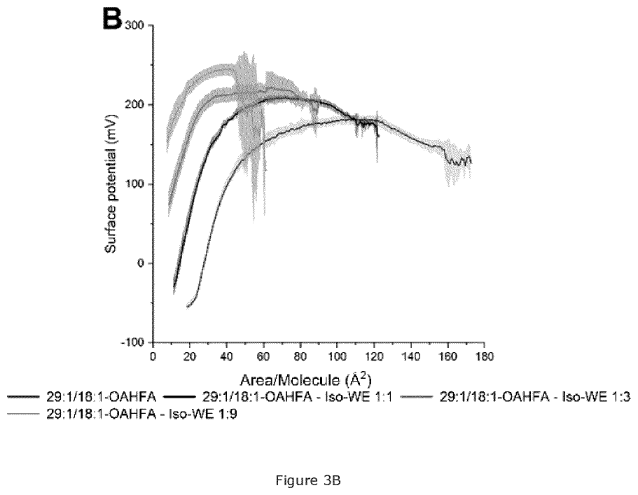

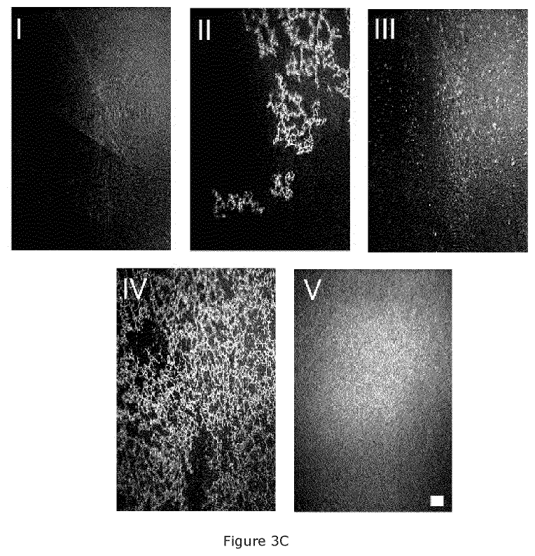

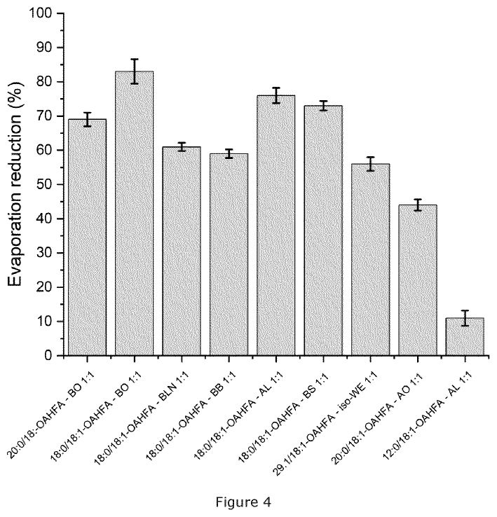

The reduction in evaporation achieved by different OAHFA:WE mixtures was measured as described in Example 10. The results are shown in FIG. 4.

While multiple promising mixed OAHFA:WE films were found, not all of them displayed improved efficacy over individual OAHFAs. The nature inspired mixture featuring iso-WE and 29:1/18:1-OAHFA as well as the previously published mixture featuring BO and 20:0/18:1-OAHFA were found to display improved efficacies over the individual components alone (compare results in FIGS. 2 and 4).

These results highlight that enhanced evaporation resistant properties are achievable through integrated WE:OAHFA monolayers. In particular, mixtures featuring the 18:0/18:1-OAHFA component were found to be superior to mixtures with other OAHFAs. In addition, only mixtures with 18:0/18:1-OAHFA and BO were able to match the properties of pure 18:0/18:1-OAHFA. When the WEs were chosen from the group of BLN, BB, AL and BS, the ability to retard the evaporation of water was found to be reduced, but was still significant. Altogether, our assessment shows that specific mixed OAHFA:WE films can be tailored to produce enhanced anti-evaporative features and that the most promising option from this category is the 18:0/18:1-OAHFA:BO 1:1-mixture.

### Long-Chain Fatty Alcohols

The evaporation resistant properties of long-chain fatty alcohols have previously been reported (Bland et al. Langmuir 2019, 35, 3545-3552). Other types of long-chain fatty alcohols have also in the past been used to retard the evaporation from water reservoirs (Gugliotti et al., J. Braz. Chem. Soc. 2005, 16, 1186-1190). In general, the properties of long-chain fatty alcohol analogues of OAHFAS (OAHFAIs) are less promising than those of the corresponding OAHFAs. Nevertheless, these species, and fatty alcohols in general, offer certain advantages. In particular, due to the polar head group and the lipid tail of fatty alcohols their inclusion in liposomes were thought to offer further tailoring possibilities in case challenges with the hydrophobic nature of the wax esters would arise. In addition, the production costs of commercially available long-chain fatty alcohols may present an economically viable option for composition development.

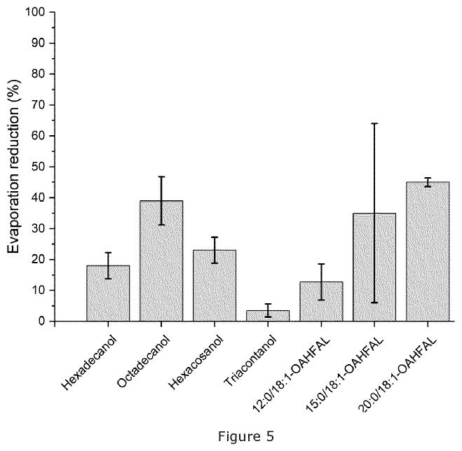

Accordingly, the evaporation resistance of a range of long chain fatty alcohols was evaluated following the procedure described in Example 10. The results of this screen are shown in FIG. 5. In terms of evaporation resistance, the most promising species was 1-octadecanol and this compound was selected for further evaluation.

### Example 12—Development of Functional Formulations

The evaporation-resistant lipids are too lipophilic to be used in simple aqueous formulations, such as many commercial eye drop formulations. Therefore, the ingredients must be formulated in a way so as to solubilize these ingredients and form a stable composition. With this in mind, there are three main options for formulation development: water-in-oil emulsions, oil solutions and liposomal formulations.

The properties of the active ingredients may also be disrupted when they are present in combination with other ingredients in formulations. Accordingly, further studies were performed to identify viable formulations for the delivery of evaporation resistant ingredients to the eye surface, i.e, for generation of an evaporation resistant film. Such, formulations need to fulfill criteria 1) and 2), discussed above i.e. they need to be able to form a uniform homogenous and tightly packed lipid film on the aqueous surface which displays evaporation resistant properties if they are to be successful in targeting the tear film instability defect associated with DED.

With this in mind, we initiated the development of formulations by assessing the properties of active components in oil-in-water emulsions (Example 7), oil solutions (Example 8) and liposomal formulations (Example 9). In our initial studies, the oil-in-water emulsions, such as the composition described in Example 7 did not form a cohesive monolayer on the aqueous surface, the properties of the oil solution were not affected by the inclusion of minor amounts of the active components, and the liposomal formulations featuring short phospholipids did not initially lead to the generation of an evaporation resistant film.

These trials highlighted the difficulties in reaching functional compositions that fulfil criteria 1) and 2)—in fact, to the best of our knowledge, there is no data in the literature on the development of functional formulation which fulfil these two criteria based on the active components presented herein. While our early trials were unsuccessful, we tailored a development program for optimizing the performance of liposomal formulations which we considered to be the most promising of these alternatives.

### Selecting Appropriate Components for Liposomal Formulations

Phospholipids were selected as the additional component in the liposomal formulations. In such a case, both the active evaporation-resistant components and the phospholipids included in the formulation are similar to the natural substrates of the tear film which is a clear advantage.

However, it is important to note that the phospholipids themselves do not form monolayers which are effective in preventing the evaporation of water (Rantamäki et al. Investig. Ophthalmol. Vis. Sci., 2012, 53, 6442-6447). Not only do they not form evaporation resistant monolayers on their own, they have likewise been shown to reduce the evaporation resistant properties when mixed with compounds displaying such features (Rantamäki et al. Investig. Ophthalmol. Vis. Sci., 2012, 53, 6442-6447). Therefore, the development of functional formulations featuring phospholipids presents considerable difficulties.

In order to understand the boundaries for the development of liposomal formulations, we started by screening the spreading behavior and evaporation resistant properties of mixtures of active components and phospholipids in chloroform. While these studies were performed in order to select a suitable phospholipid component, they simultaneously showcase the physical mechanism of action of the compositions-once the components have been delivered to the ocular surface. The screening of phospholipids was carried out using the 18:0/18:1-OAHFA species as a representative example of the active compounds.

Three separate 1:1 18:0/18:1-OAHFA:phospholipid mixtures employing the following phospholipid components: DMPC, DSPC and DAPC were evaluated. In all three cases, the evaporation resistant properties of the 18:0/18:1-OAHFA were found to be diminished when used in combination with the phospholipids. However, a considerable degree of evaporation resistance was retained with certain phospholipids. In particular, the mixtures containing longer phospholipid species were found to be better at retaining the evaporation resistant properties than shorter species and a clear trend can be seen in our series. Of the species tested, DMPC reduced the evaporation-resistant properties of the 18:0/18:1-OAHFA, but the mixtures with DAPC and DSPC still had considerable evaporation-resistant properties.

Without wishing to be bound by theory, it is believed that the shorter phospholipid species does not allow formation of the required monolayer structure, and instead these mixtures exist in the liquid state thus failing to comply with selection criteria 2) outlined above. However, DMPC was also taken forward for further development because it is known to have thermosensitive properties that may alter its behavior in an in vivo setting and it was found to allow higher loadings of the lipid components into the formulations.

The evaporation-resistant properties of the combination of the 12:0/18:1-OAHFA with DAPC were also assessed and found to be inferior to the combinations with the 18:0/18:1-OAHFA, thus underlining the diverse properties displayed by different OAHFA species.

Next, we focused on understanding the effect of active:phospholipid-ratios on the evaporation resistant function of the mixed films formed. These studies are exemplified using DAPC as the phospholipid and either 18:0/18:1-OAHFA or 20:0/18:1-OAHFA and BO as the active components. In the case of the 18:0/18:1-OAHFA, changing the active:phospholipid ratio in the range of 1:1 to 1:3 did not have a dramatic effect on the evaporation resistant properties of the mixture whereas a small decrease in the evaporation resistant properties were observed in a similar series featuring the other OAHFA:WE-combination.

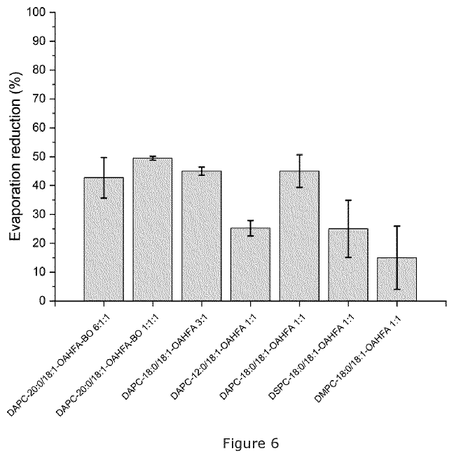

The results of these studies are shown in FIG. 6.

### Preparation of Liposomal Formulations

Liposomal formulations can be prepared by a number of different techniques. Here, we employed the thin film hydration method as disclosed in Example 9. While this technique is generally considered a simple and straight-forward protocol for the preparation of liposomes, a number of challenges were encountered at distinct steps due to the properties displayed by the active components and the phospholipids. These factors highlight that the development of functional formulations is not a trivial task-even when a tailored and comprehensive pre-screening platform has been employed during the selection process.

We first addressed the total lipid concentrations allowed in distinct liposomal formulations. With DMPC, the formulation protocol worked seemingly well and formulations with a total lipid concentration up to 15-17% could be produced without encountering problems with viscosity. With DSPC and DAPC on the other hand, the hydration step was found to be time-consuming, the sonication step was complicated by foaming and the end formulations were found to be too viscous at these concentrations. Nevertheless, these issues could be resolved by lowering the total lipid concentration in the formulations.

Altogether, this screen indicated that the total lipid concentrations allowed are approximately 15-17% for DMPC, 8% for DSPC and 3% for DAPC. The trend uncovered is that an increase in the chain length of the phospholipid is accompanied by a decrease in the total lipid concentration sustainable in such a formulation. Nevertheless, considering the intended physical mechanism of action of the active components—the total lipid concentration in the formulations does not need to be considerable to achieve the desired effects. On the contrary, a large number of liposomal formulations were prepared (see example 9 and Table 1).

### Characterization of Liposomal Formulations

The liposomal formulations prepared were characterized by a number of experimental techniques. DSC was utilized to verify that the active components were infused with the phospholipids in the liposomes and important properties such as the zeta potential and particle size were determined by conventional nanoparticle tracking analysis. In addition, the pH and osmolality of the formulations were assessed.

### DSC Analysis

Differential scanning calorimetry (DSC 2500 with an RCS90 cooling unit; TA instruments, Newcastle, DE, USA) was utilized to study the thermal properties of the active compounds. Nitrogen (N₂) was utilized as the purge gas at rate of 50 mL/min during the measurements. 0.5-2 g of the active components were placed into aluminum pans with manually made pin holes (DSC Consumables incorporated, Austin, Minnesota, USA). The samples were subsequently sealed and analyzed with DSC utilizing the TRIOS® software (TA instruments, Newcastle, DE, USA). All results were compared to an empty reference pan containing a pin hole. Each measurement was performed in triplicate. Briefly, the samples were cooled to 0° C., then heated from 0° C. to 75° C. at a rate of 10° C./min. Then, the samples were kept isothermally at 75° C. for 10 min, after which they were equilibrated to −50° C. at an uncontrolled rate. Ultimately, the samples were heated again to 75° C. at a rate of 10° C./min.

The phospholipid bilayer phase transition behavior was studied to assess whether successful integration of the active components into the phospholipid bilayer had been achieved or not. The studies were performed in a similar fashion as described for active components above. In this case, 20 μL of a formulation was pipetted into the aluminum pans (DSC Consumables incorporated, Austin, Minnesota, USA), which were subsequently sealed and analyzed with DSC employing the TRIOS® software (TA instruments, Newcastle, DE, USA). The samples were cooled and equilibrated to 5° C., then heated from 5° C. to 70-85° C. at a rate of 10° C./min and the results were compared to the reference pan containing the liquid used to hydrate the lipid film. All prepared liposomal formulations which viscosity were low enough to be pipetted, were analyzed by DSC and compared to the thermograms obtained from liposomes containing only the PC-component and the thermograms obtained for the active components. When the phase transition temperatures observed were found to follow a similar pattern as for the phospholipids and the phase transition peaks of the active components were absent—the conclusions reached were that the active components had been successfully integrated within the phospholipid bilayer.

Prior to the assessment of liposomes by DSC, the phase transitions of the most promising active components (20-OAHFA, 18-OAHFA, R-18-OAHFA, BO, octadecanol) were determined. All of the active compounds induced clear exothermic peaks upon crystallization and endothermic peaks upon melting. For 20-OAHFA and R-18-OAHFA, two separate peaks were observed close to each other upon melting and these were designated Tₘ1 and Tₘ2. The melting temperatures for the active components are given in Table XY. It is important to note that the Tₘ1-values for the most promising active components i.e. 18-OAHFA, R-18-OAHFA and octadecanol are almost identical. In a similar fashion, the endothermic gel-to-liquid phase transition peaks for the pure phospholipids were determined (see table 2).

TABLE 2
Melting temperatures of active compounds and gel-to-liquid
phase transition temperatures of phospholipids.

| | Onset Tₘ1 ± | Peak Tₘ1 ± | Onset Tₘ2 ± | Peak Tₘ2 ± |
|---|---|---|---|---|
| Component | SD | SD | SD | SD |
| 20-OAHFA | 41.65 ± 1.16 | 46.54 ± 0.18 | 56.89 ± 1.42 | 59.52 ± 0.38 |
| 18-OAHFA | 54.92 ± 0.87 | 56.50 ± 0.64 | | |
| R-18-OAHFA | 55.00 ± 0.87 | 57.53 ± 0.95 | 63.20 ± 0.28 | 63.88 ± 0.14 |
| BO | 34.48 ± 0.05 | 36.74 ± 0.92 | | |
| 1-Octadecanol | 56.49 ± 0.49 | 57.61 ± 0.22 | | |
| DMPC | 17.37 ± 0.69 | 22.74 ± 1.57 | | |
| DSPC | 50.13 ± 0.29 | 54.67 ± 0.18 | | |
| DAPC | 63.04 ± 0.21 | 64.87 ± 0.05 | | |

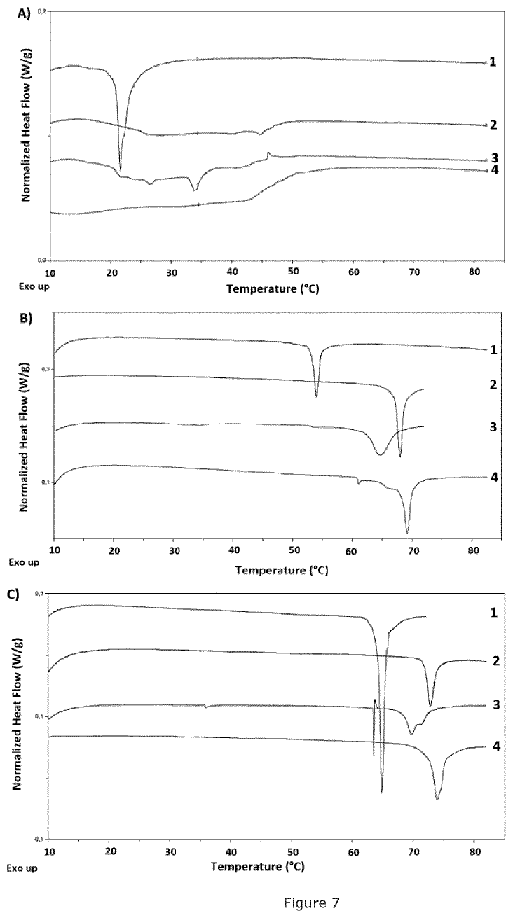

During the characterization of liposomal formulations, a failure to incorporate all of the active components could be distinguished from the DSC-thermograms as the characteristic peaks of the individual components would still be present. Moreover, with the majority of formulations analyzed, only one endothermic peak appeared in the thermograms and these were located close to the phase transition temperature of the employed phospholipid. While so, the shape and width of the peak was found to vary depending on the phospholipid, active component and the ratio between these (see FIG. 7A and B).

With DMPC-based formulations containing active components, a substantial peak broadening was observed in the thermograms. In fact, increased peak broadening was found to correlate with an increase in active component:phospholipid-ratios. Nevertheless, the Tₘ onset values for the gel-to-liquid phase transitions remained similar in the series studied and individual peaks for the active components could not be detected in active component:phospholipid ratios up to1:4 suggesting that the active species are fully incorporated into the liposomal structure. Therefore, the broadening of the peaks was accepted as describing the complex thermal behavior of DMPC-based liposomal formulations.

The DSPC and DAPC-based formulations displayed a distinct behavior from the DMPC-based ones. While we were originally concerned about the verification possibilities of unincorporated active components in the DSPC-formulations due to Tₘ-values in the same range for both individual species, these concerns proved unwarranted as the phase transition temperature of liposomes containing active components were sufficiently shifted to allow detection of the Tₘ-melting peaks for individual components. Altogether, peak broadening was not observed in the DSC-thermograms to a similar extent (although a correlation indicating a dependence on active components could be observed) as in the case for DMPC thus allowing a straight-forward way of interpreting the results. In addition, changes in active components and or active components:phospholipid-ratios resulted in similar alteration trends in the phase behavior of these formulations. These trends will be highlighted by going through a few selected examples.

With both DSPC- and DAPC-based liposomes, phospholipid:active component mass ratios of 2:1 (molar ratios of approx. 1.4:1) were tolerated when the active component was selected from the OAHFA-category (18:0/18:1-OAHFA). When the active component was selected from the OAHFA:WE-category (18:0/18:1-OAHFA or R-18:0/18:1-OAHFA and BO), the tolerated mass ratios were not as high as small amount of unincorporated BO could be observed in the thermograms. For the sake of comparison, also liposomes featuring a long-chain fatty alcohol (octadecanol) and mixtures of a OAHFA:long-chain fatty alcohol (18-OAHFA:octadecanol) or a long-chain fatty alcohol:WE (octadecanol:BO) was analyzed by DSC. Based on these studies, a few trends could be uncovered. First, inclusion of WE-species results in widening of the endothermic peak in the thermograms. Second, a trend pointing towards a correlation between loading capacity and active component selection was uncovered. In more detail, a decrease in loading capacity was found when a WE-species was included as part of the active component. The DSC-analysis of different formulation proved to be an effective tool for evaluating the infusion of the active components in the phospholipid bilayer and mapping the boundaries within which the successful creation of liposomal formulations can be undertaken.

### Nanoparticle Tracking Analysis (NTA) and Assessing the pH and Osmolality of Formulations

In addition to performing a thorough analysis of the liposomes by DSC, other important properties such as osmolality, pH, particle size and zeta potential were likewise studied. In these studies, clear trends could not be observed for the liposomal formulation based on the utilized phosphatidylcholine (DMPC, DSPC, DAPC).

### Nanoparticle Tracking Analysis

The size and zeta potential of the liposome particles were measured with ZetaView® Nanoparticle Tracking Analyzer PMX-120-Z-520-F (Particle Metrix GmbH). For each measurement, 11 cell positions were scanned and 30 frames captured under camera sensitivity of 80 and shutter of 50. In case abnormalities were detected in any of the 11 cell positions during the measurement, the individual cell position was removed from the final analysis. The suitable sensitivity for the measurements was selected by utilizing the software function “Number of Particles vs. Sensitivity”. Formulation dilutions were made in a ratio were the scattering intensity and number of detected particles in one frame were at appropriate level (scattering intensity <8 and the number of detected particles 50-400). Accordingly, the samples were diluted 1:250000-1:1000000 with Milli-Q® ultrapure water (Millipore, Bedford, MA, USA) and injected into the measuring chamber. All measurements were performed at rt. After the video capture, the videos were analyzed by the in-built ZetaView Software (version 8.05.14). The post-acquisition parameters (i.e., the digital filters applied to images) for the analysis were set as follows: Maximum area 1000, minimum area 5, and minimum brightness 20.

### Assessing pH and Osmolality

Visual inspection was used to assess the overall potential of all prepared formulations, and formulations displaying poor properties were not further screened. The most eligible formulations were further characterized in terms of pH, surface tension, and osmolality. The pH of the formulations was measured with ORION SA 520 pH meter (Orion Research Incorporated, Boston, USA) and auto-calibrated with pH 4 and pH 7 buffer solutions, while the osmolality of the formulations was measured with Osmostat® OM-6020 Auto-Osmometer (Daiichi Kagaku, Kyoto, Japan), which was calibrated with Milli-Q water and 300/1000 mOsm/kg standard solutions.

### Results

When the lipid film was hydrated in water, the pH of the liposomal formulations was found to be in the 3.5-5.7 range and the osmolality in the 4-28 mOsm/kg range. These values are not optimal for topical administration to the ocular surface or eye. In order to reach a more biocompatible formulation, the lipid film was hydrated in a TBS-buffer (20-50 mM, pH 7.4). This resulted in formulations with neutral pH-values and osmolality in the 339-363 mOsm/kg range. Therefore, the pH and osmolality of the formulations are adjustable to meet the requirements related to topical administration of these to the ocular surface/eye.

The zeta potential of the liposomes varied between negatively charged and neutral. However, a clear trend between the selection of active components, phospholipids and the resulting zeta potential values was not observed. Nevertheless, the average size of the liposomes were found to vary slightly within the 120-200 nm range with more than 90% of all particles bearing a size below 300 nm.

Altogether, the liposomal formulations prepared were subjected to wide range of characterization studies in order to assess their structural integrity and basic properties. Liposomes infused with a number of distinct active components were successfully prepared and characterized. Moreover, the liposomes could be tailored to comply with the requirement of topical administration to the ocular surface/eye. With access to a number of well-characterized liposomal formulations, we continued by assessing the functional properties of these.

### Example 13—Biophysical Profiling of Liposomal Formulations

Biophysical profiling of the liposomal formulations is crucial to assessing whether their abilities are sufficient to adhere to requirements 1) and 2) outlined at the beginning of the formulation development, characterization and assessment section. The liposomal formulations need to be able to deliver the active components in functional form to the ocular surface. In other words, the liposomes need to be able to induce the formation of a uniform homogenous densely packed lipid film with evaporation resistant properties at the aqueous interface.

In addition, the condensed lipid film needs to display adaptive features i.e. it needs to be capable of respreading at the aqueous interface over eye blink cycles. These three factors were the focus of the biophysical profiling of liposomal formulations. The techniques used in the assessment are identical to the ones used during the selection of active components phase and the details are given in Example 10.

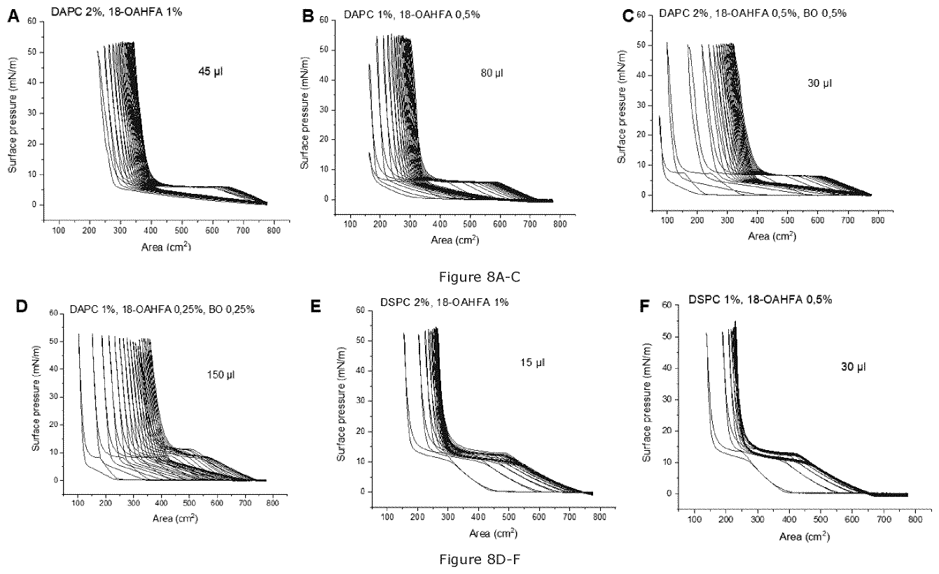

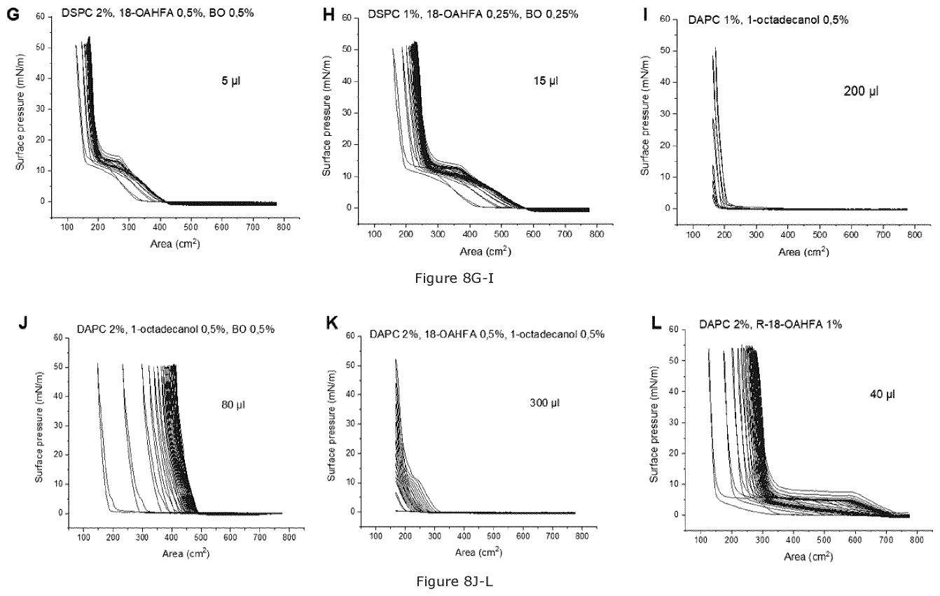

The behavior of the liposomal formulations was assessed over compression-expansion cycles. This experimental setup models the changes in surface pressure occurring during an eye blink cycle and can be utilized to gain insights on the adaptive features of the film formed. In FIG. 8 (A to L), the changes in surface pressure isotherms over compression-expansion cycles for a number of formulations are shown, along with the pipetted volume employed of each formulation.

These graphs should be interpreted in the following way: The volumes required for each of the formulations is connected to the surface activity displayed by said formulation. In other words, a small volume indicates that the lipids are efficiently delivered through the formulation to the aqueous subphase while a large volume indicates challenges in lipid delivery. During the compression-expansion cycles, a notable shift in the surface pressure isotherms (from small areas to larger areas) can be observed. A shift to large areas is advantageous as it indicates that the film formed can cover a larger area. Altogether, the results provide insights on the delivery of lipid components as a function of compression-expansion cycles and time, and, indicates which formulations are most efficient in covering a larger surface area. It should be noted that the surface pressure isotherms follow a similar profile as that of the active components which can be taken as evidence of their involvement in the film formed. From FIG. 8, it is clear that distinct formulations display distinct character. For example, formulations D, I and K require substantially larger volumes (150-300 μl vs. 5-80 μl for the rest) in order to shift the surface pressure isotherms to larger areas and for formulations I and K even a larger volume does not lead to a desired shift in the isotherm. Considering that one drop of water has a volume of ˜50 μl and the area of the Langmuir-Blodgett through is considerably larger than the ocular surface area, all formulations except for D, I and K should still be applicable in the intended application. Most importantly, the adaptability displayed by the formed lipid films is significant-relatively small hysteresis can be observed throughout the compression-expansion cycles and after a set number of cycles the resulting lipid film behaves in an identical fashion. In other words, once the delivery of lipids has been completed, the film formed is capable of efficiently respreading over compression-expansion cycles. An important feature for a promising eye treatment based on our intended mechanism of action.

In addition to profiling the behavior of the lipid films over compression-expansion cycles, we analyzed the structure of the films by Brewster angle microscopy in order to ascertain that they formed a condensed film with potential evaporation resistant properties upon dissociation of the liposomes. As detailed above, the phase transitions observed in the surface pressure isotherms were similar to the ones observed for the individual active components which provided evidence of their successful inclusion in the film formed and gave a strong indication that their functional capabilities would remain unaltered.

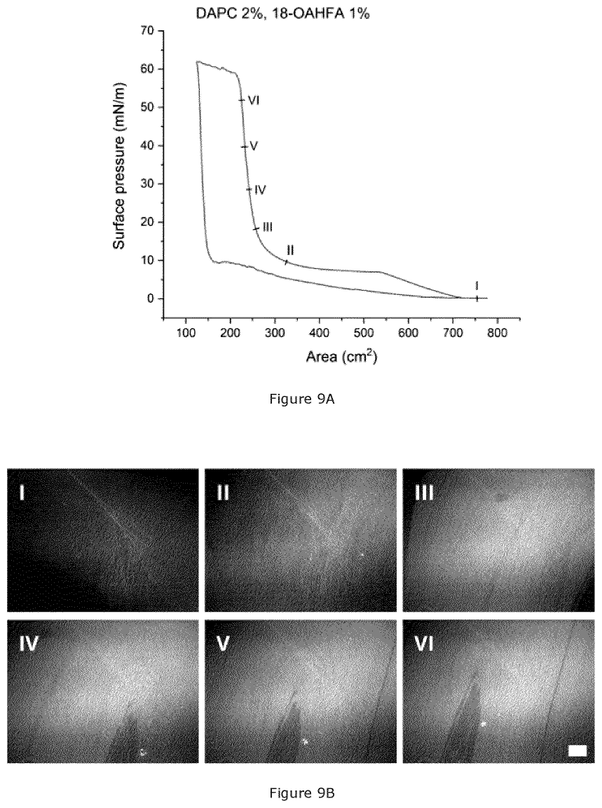

While many of the tested formulations functioned in the intended way, the properties will here be exemplified by highlighting the behavior of the lipid film formed from the formulation containing 2% DAPC and 1% 18:0/18:1-OAHFA. During the compression-expansion cycle, the film structure was imaged and it was clear that the liquid-to-solid phase transition observed was reminiscent to the one displayed by the active components alone i.e. a solid homogenous monolayer was formed (see FIG. 9). This demonstrated that the formulations form a condensed lipid film which in turn is a pre-requisite for achieving evaporation resistant properties.

### Evaporation Resistance of Liposomal Formulations

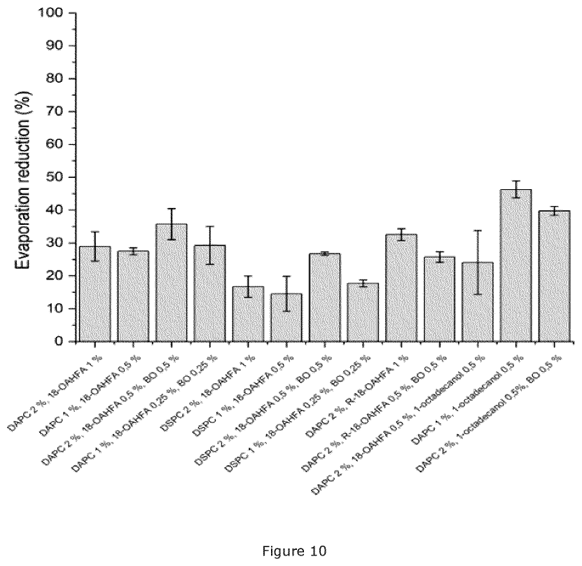

The evaporation resistant properties of a large number of liposomal formulations was then assessed according to the protocol outlined in Example 10 (representative examples shown in FIG. 10). The formulations developed through our biophysical screening platform displayed evaporation resistant properties which indicates that the criteria developed for the selection stage are valid. For the most promising formulations, a 25-40% reduction in water evaporation was observed. A few trends can be observed based on the data. First, the evaporation reduction values reached for the formulations are lower than the corresponding values for the active component solutions as such. Second, the DAPC-based formulations displayed higher evaporation resistance than the DSPC-based ones across the categories of active components studied.

Altogether, the results demonstrate the substantial amount of screening and profiling required to develop successful evaporation resistant formulations which fulfill criteria 1) and 2). We have been able to develop such formulations from three distinct active component categories by careful tailoring of liposomal structures. While the developed formulations display promising biophysical properties, we proceeded to determine if any of the formulations would have clear advantages from a biological perspective as well through assessing their performance in in vitro cytotoxicity and efficacy studies.

### Example 14—In Vitro Cytotoxicity and Efficacy Assessment of Liposomal Formulations

In order to determine the toxicity profiles of the formulations, the cytotoxicity of the formulations was evaluated according to the following procedure. For reference, two commercial formulations (Oxyal® triple action and Systane® complete) for the treatment of dry eye disease were also included in the studies.

Human corneal epithelial (HCE) cells were cultured at 37° C. and 5% CO₂ atmosphere in the growth medium. The growth medium consisted of DMEM/F12 1:1, 15% of FBS (Fetal bovine serum), 1% of penicillin/streptomycin, 0.3 mg/ml of L-glutamine, 10 ng/ml of EGF (epidermal growth factor, 5 μg/mL of human recombinant insulin, 0.5% of sterile filtrated DMSO (Sigma-Aldrich, St. Louis, MO, USA), 0.1 μg/ml of cholera toxin and 15 mM of HEPES (BioWhittaker Lonza, Walkersville, MD, USA). The cells were fed with growth medium every two to three days, and subcultured 1:5-1:30 or seeded for the experiment when reaching 75-80% confluence. Upon the confluence, the cells were washed with PBS and detached with 0.05% trypsin-EDTA (all materials were derived from Gibco, unless described otherwise). The toxicity of the formulation was assessed utilizing the MTT assay, a colorimetric method that addresses the mitochondrial metabolic activity of the cells by detecting absorbance that is in respect to cell viability.

In more detail, the cells were seeded in 96-well plates at a density of 20 000 cells/well and after incubating them overnight at 37° C., the cells were exposed to the formulation diluted in serum-free medium at various ratios (½- 1/64 dilutions, 150 μL/well). After incubating the cells at 37° C. for 3 hours, the medium containing formulations was aspirated, and cells were washed thoroughly with PBS. Thereafter, the cells were either treated immediately with 100 μL of 0.5 mg/ml MTT in serum free media (3-(4,5-dimethylthiazolyl-2)-2,5-diphenyl tetrazolium bromide (Sigma-Aldrich, St. Louis, MO, USA), 10% of 5 mg/ml in PBS mixed with 90% of serum-free medium) or after allowing them to recover overnight at 37° C. with only 150 μL of serum-free medium. 2 hours after the addition of MTT-media solution, 100 μL of dodecyl sulfate sodium salt-N,N-Dimethylformamide (SDS-DMF) lysis buffer (pH 4.7, 200 mg/ml SDS from Sigma-Aldrich, St. Louis, MO, USA in DMF:H2O 1:1, Gibco, Thermo Fisher Scientific, Waltham, MA, USA) was added to the wells with further incubation overnight. The absorbance was measured at 570 nm by a Victor 2 multilabel plate reader (PerkinElmer, Wallac, St. Paul, MN, USA). The percentage of cell viability was calculated as addressed in equation 3. Wells that consisted only of MTT-solution and SDS-DMF lysis buffer served as blanks and were subtracted from all samples. Wells with control cells that were not exposed to formulations denoted the reference level of 100% cell viability, on which further comparisons and the % of cell viability were based on (equation 3).

$$Cell viability ( \% ) = ( {A}_{Sample} - {A}_{Blank} ) / ( {A}_{Control} - {A}_{Blank} ) * 100 \qquad ( 3 )$$

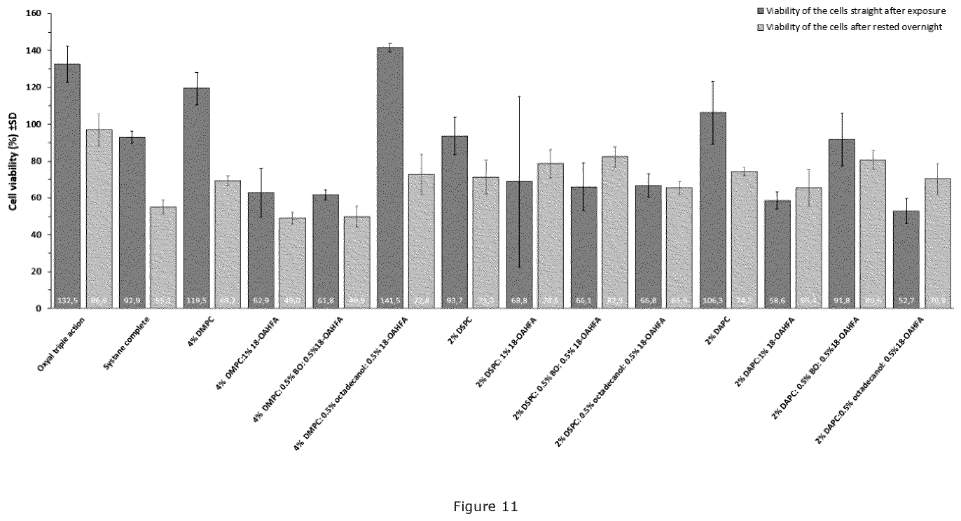

In separate experiments, after a three-hour exposure to the formulation, we measured the viability of the ocular surface cells immediately, in the first experiment, and, in the second experiment, after an overnight incubation in serum free media. The results are summarized in FIG. 11. While some decrease in cell viability was initially detected after the three-hour exposure time compared to the commercial medications, when viability was determined from the cells that rested overnight after the exposure, the cell viability values were found to be within the range displayed by the commercial products.

### Example 15—In Vitro Biological Efficacy Study

The biological efficacy of the formulations was assessed with HCE cells by the widely employed BAC model, in which the cells were initially exposed to benzalkonium chloride (BAC), which is known to be toxic and induce corneal epithelial cell damage (Saarinen-Savolainen P, Jäarvinen T, Araki-Sasaki K, Watanabe H, Urtti A. Evaluation of cytotoxicity of various ophthalmic drugs, eye drop excipients and cyclodextrins in an immortalized human corneal epithelial cell line. Pharm Res 1998; 15 (8): 1275e80, Cha S H, Lee J S, Oum B S, Kim C D. Corneal epithelial cellular dysfunction from benzalkonium chloride (BAC) in vitro. Clin Exp Ophthalmol. 2004 April;32 (2): 180-4. Doi: 10.1111/j.1442-9071.2004.00782.x., Hakkarainen J J, Reinisalo M, Ragauskas S, Seppänen A, Kaja S, Kalesnykas G. Acute cytotoxic effects of marketed ophthalmic formulations on human corneal epithelial cells. Int J Pharm. 2016 Sep. 10; 511 (1): 73-78. Doi: 10.1016/j.ijpharm.2016.06.135.). After approximate BAC-induced cell death, the cells were treated with formulations to observe if the cell recovery was improved when treated with formulations. Two commercial dry eye medications (Oxyal® triple action and Systane® complete) were used as references in these studies.

The cell viability was determined by the MTT assay and the experimental setup was similar to the one described in Example 14. After incubating the cells overnight at 37° C. in a 96-well plate, the cells were first exposed to 0.001% BAC for 45 min, and then treated with formulations diluted in serum-free media. The experiment was conducted by using several formulation dilutions (¼- 1/32) and two different treatment times (six and 24 hours). After incubating the cells at 37° C. for 24 hours (counting from BAC exposure), the MTT assay was conducted. Again, the wells consisting of only reagents served blanks, and control cells that were not exposed to BAC nor formulations denoted the reference level of 100% cell viability. The cells that were only exposed to BAC but not treated with formulation provided a baseline level to assess whether formulation treatment improved cell recovery. In addition, the cells that were exposed to BAC and cultured in full growth medium containing 15% FBS served as a positive control, since FBS contains many growth promoting factors which enhance cell recovery.

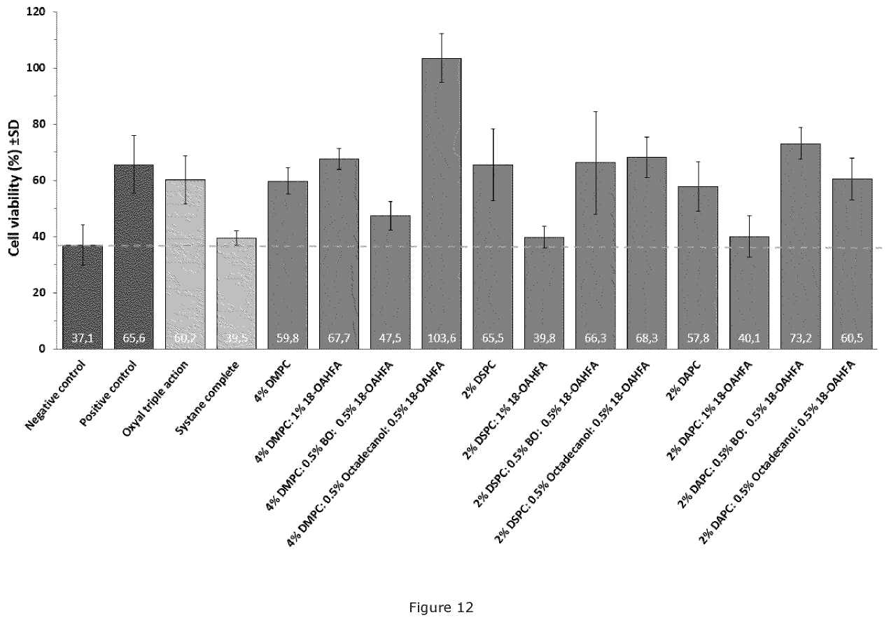

In this experimental setup BAC-exposed cells cultured in serum-free medium without formulation treatment were used as a negative control and BAC-exposed cells cultured in full growth media containing 15% of FBS were used as a positive control. The results are summarized in FIG. 12. Altogether, the majority of our formulations improved the recovery of HCE cells after initial BAC-exposure. The results prove that our formulations enhance the recovery of HCE cells. In fact, they were found to be equally effective or superior to the commercial dry eye medications on the market in this assay.

### Example 16—Further Screening of Phospholipids and Lipid-Phospholipid Combinations

To investigate the effects of the choice of phospholipid on the evaporation resistant and other properties of the compositions, a range of phospholipids were assessed for their surface activity and evaporation resistance according to the protocols described above. Then, selected phospholipids from this screen were tested as 1:1:1 molar mixtures with 20:0/18:1-OAHFA (20-OAHFA) and BO to determine their effects on the properties of the evaporation resistant compositions.

### Screening of Phospholipids

The properties of the following phospholipids were assessed.
1-palmitoyl-2-hydroxy-sn-glycero-3-phosphocholine (16:0 Lyso PC)
1,2-dilauroyl-sn-glycero-3-phosphoethanolamine (12:0 PE)
1,2-dimyristoyl-sn-glycero-3-phosphoethanolamine (14:0 PE)
1,2-dipalmitoyl-sn-glycero-3-phosphoethanolamine (16:0 PE)
1,2-distearoyl-sn-glycero-3-phosphoethanolamine (18:0 PE)
1,2-ditetradecanoyl-sn-glycero-3-phospho-(1′racglycerol) (sodium salt) (14:0 PG)
1,2-distearoyl-sn-glycero-3-phospho-rac-(1-glycerol) sodium salt (18:0 PG)
1,2-distearoyl-sn-glycero-3-phospho-L-serine (sodium salt) (18:0 PS)
1,2-dilauroyl-sn-glycero-3-phosphate (sodium salt) (12:0 PA)
1,2-dipalmitoyl-sn-glycero-3-phosphate (sodium salt) (16:0 PA)
1,2-distearoyl-sn-glycero-3-phosphate (sodium salt) (18:0 PA)

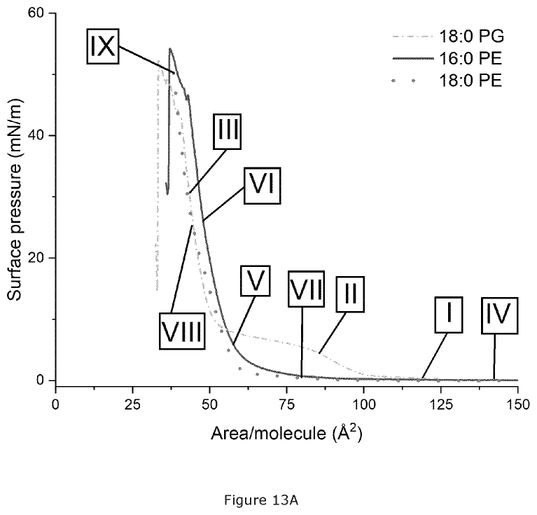

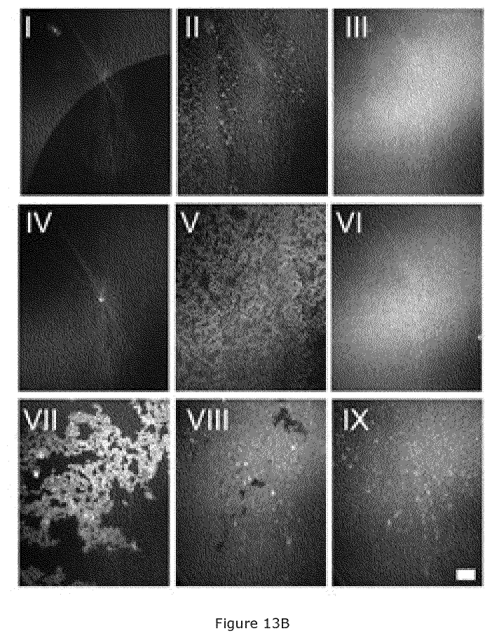

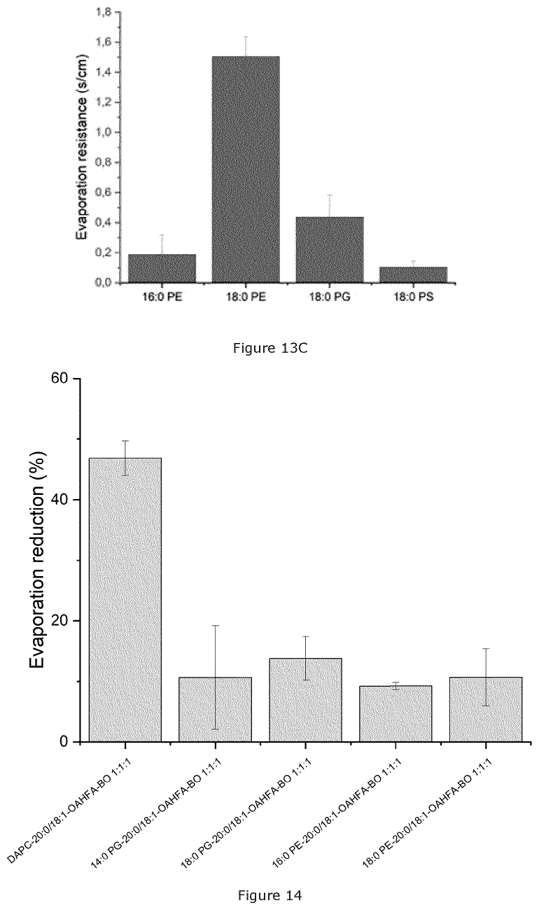

The majority of the tested lipids were in a liquid phase at conditions mimicking those at the ocular surface and are thus not expected to have useful evaporation resistant properties. Only four potential phospholipids with suitable properties were identified based on the BAM-imaging. These were 16:0 PE; 18:0 PE, 18:0 PG and 18:0 PS. Out of these four, films formed by 18:0 PG and 18:0 PE displayed the highest evaporation resistance (FIG. 13C). While 18:0 PE displayed the highest evaporation resistance, the phase transition occurring at a low surface pressure in 18:0 PG (as can be seen from FIG. 13B, images I, II and III, which show that 18:0 PG forms a homogenous film at low pressure (image I) and undergoes a phase transition to a more solid film at higher pressures (images II and III) might be favorable from the formation of a homogenous film perspective. Thus these two phospholipids could have the potential to have similar properties to DSPC and DAPC when used with a lipid component in the evaporation-resistant formulations.

FIG. 13A shows surface pressure isotherms of 18:0 PG, 16:0 PE and 18:0 PE. The Roman numerals I to IX correspond to the BAM images shown in FIG. 13B.

### Lipid-Phospholipid Combinations

The evaporation reduction of the following lipid-phospholipid combinations was assessed.
DAPC: 20-OAHFA:BO (1:1:1)
14:0-PG: 20-OAHFA:BO (1:1:1)
18:0-PG: 20-OAHFA:BO (1:1:1)
16:0-PE: 20-OAHFA:BO (1:1:1)
18:0-PE: 20-OAHFA:BO (1:1:1)

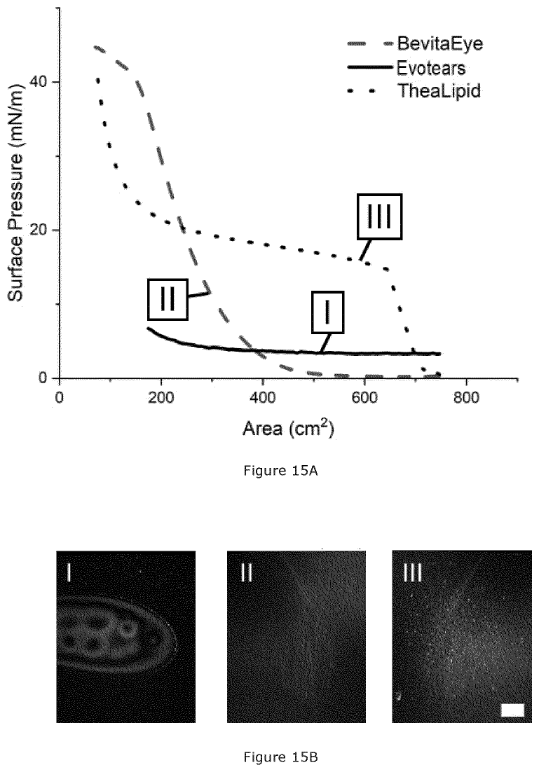

The DAPC: 20-OAHFA:BO combination, surprisingly, achieved by far the highest degree of evaporation reduction, of over 40%. The combinations with 18:0-PG and 18:0-PE displayed some evaporation reduction, but, despite these phospholipids having promising properties for inclusion in evaporation resistant formulations, the combinations containing these compounds achieved significantly lower evaporation reduction than the combination with DAPC. The combinations with 14:0-PG and 16:0 PE achieved similar levels of evaporation reduction to the 18:0-PG and 18:0-PE combinations. The results of this experiment are shown in FIG. 14.

### Example 17—Evaporation Resistance of Commercial Dry Eye Formulations

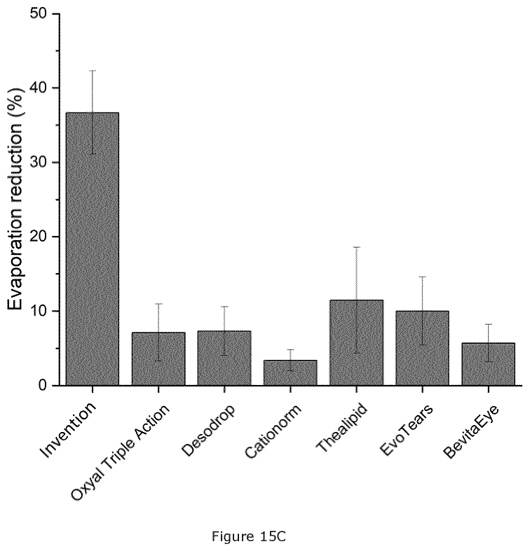

A range of commercially available eye drops (Oxyal Triple Action, Desodrop, Cationorm, Thealipid, EvoTears, BevitaEye) were analysed for their surface activity and evaporation reduction following the procedures described in Example 10. The screened products generally displayed good surface activity. Only the phase behaviour of EvoTears was unusual. Evotears contains a fluorinated hydrocarbon as an active ingredient which does not interact with the aqueous phase as can be seen from the surface pressure isotherm (FIG. 15A). In addition, Evotears did not form a homogenous lipid film (as for example BevitaEye and Thealipid, FIG. 15B). Instead, big droplets were formed on the aqueous phase indicating a poor spreading capability.

The absolute maximum evaporation reduction values for commercial products were found to be in the range of 6-15% (FIG. 15C) which is significantly below that of the formulations of the present invention (Exemplified in FIG. 15C by a liposomal formulation containing 2% DAPC, 1% 20-OAHFA and 1% behenyl oleate (labelled as ‘Invention’). 5

The relatively low evaporation resistance of the commercial formulations was believed to be due to the lack of formation of solid or gel-like phases which is a prerequisite for reaching higher reduction of water evaporation values. These results indicate that the compositions of the invention described herein can offer significant benefits compared to currently-marketed treatments for ocular disorders, such as dry eye disease.

1. A pharmaceutical composition comprising liposomes, which liposomes comprise:
(a) a lipid component selected from the group consisting of:
(i) a fatty acid ester of a hydroxy fatty acid of formula I or a pharmaceutically acceptable salt thereof, 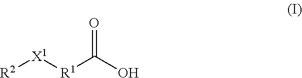
wherein:
R¹ is selected from a C₁₆-C₃₀ alkanediyl or a C₂₁-C₃₀ alkenediyl group containing one double bond;
R² is selected from a C₁₅-C₁₉ alkyl group or a C₁₅-C₁₉ alkenyl group containing one or two double bonds; and
X¹ represents an ester group selected from the group consisting of: 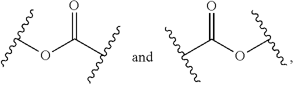
wherein [symbol] represents a point of attachment to the rest of the molecule;
(ii) a combination of a fatty acid ester of a hydroxy fatty acid of formula I, or a pharmaceutically acceptable salt thereof, as defined in (i), and a wax ester of formula II 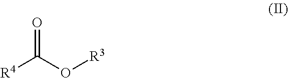
wherein:
R³ represents a C₁₈-C₃₀ alkyl group, and
R⁴ represents a C₁₀-C₂₄ alkyl group or a C₁₀-C₂₄ alkenyl group containing one or two double bonds;
wherein the fatty acid ester of a hydroxy fatty acid of formula I and the wax ester of formula II are present in a molar ratio of from about 2:1 to about 1:9;
(iii) a fatty alcohol of formula III, or a pharmaceutically acceptable salt thereof, 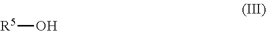
wherein R⁵ represents a C₁₄-C₃₀ linear or branched alkyl group; or
formula IV, 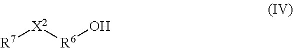
wherein:
R⁶ is selected from a C₁₀-C₃₀ alkanediyl or a C₁₀-C₃₀ alkenediyl group containing one double bond;
R⁷ is selected from a C₁₄-C₁₉ alkyl group or a C₁₄-C₁₉ alkenyl group containing one or two double bonds; and
X² represents an ester group selected from the group consisting of 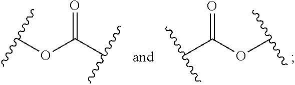 [SMILES: *OC(*)=O ; *OC(*)=O]
(iv) a combination of a fatty acid ester of a hydroxy fatty acid of formula I, or pharmaceutically-acceptable salt thereof with a fatty alcohol of formula III or IV, or a pharmaceutically acceptable salt thereof, wherein the components are present in an mass ratio of from about 3:1 to about 1:3; or
(v) a combination of a wax ester of formula II with a fatty alcohol of formula III or IV, or a pharmaceutically acceptable salt thereof, wherein the components are present in a mass ratio of from about 3:1 to about 1:3; and
(b) a phospholipid component, which is selected from the group consisting of a phosphatidylcholine, a phosphatidylglycerol, or a pharmaceutically acceptable salt thereof, a phosphatidylserine, or a pharmaceutically acceptable salt thereof, a phosphatidic acid, or a pharmaceutically acceptable salt thereof, a phosphatidylethanolamine and a mixture thereof; wherein the lipid component and phospholipid component are present, in the composition, in a ratio of from about 10:1 to about 1:10 by mass.

2. A composition as claimed in claim 1, wherein R¹ is a C₁₇-C₂₄ alkanediyl group or a C₂₄-C₂₉ alkenediyl group containing one double bond; optionally wherein R¹ is a C₁₇-C₂₂ alkanediyl group or a C₂₇-C₂₉ alkenediyl group containing one double bond.

3. A composition as claimed in claim 1, wherein R¹ is a C₁₇-C₁₉ linear alkanediyl group.

4. A composition as claimed in claim 1, wherein X¹ represents 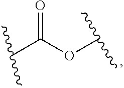 [SMILES: *OC(*)=O]
and/or R² represents a C₁₇ alkenyl group containing one or two double bonds, optionally wherein R² represents

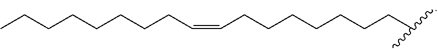 [SMILES: *CCCCCCC/C=C\CCCCCCCC]

5. A composition as claimed in claim 1, wherein X¹ represents 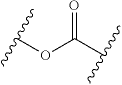 [SMILES: *OC(*)=O]
and/or
R² represents a C₁₈ alkenyl group containing one or two double bonds, preferably wherein R² represents

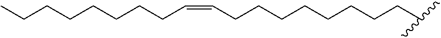 [SMILES: *CCCCCCCC/C=C\CCCCCCCC]

6. A composition as claimed in claim 1, wherein R³ represents a C₁₈ to C₂₆ alkyl group, preferably wherein R² represents a C₁₈ to C₂₂ alkyl group; and/or R⁴ represents a C₁₁ to C₂₂ alkyl or a C₁₁ to C₂₂ alkenyl group containing one or two double bonds.

7. A composition as claimed in claim 1, wherein R⁵ represents a C₁₆ to C₂₆ alkyl group, preferably wherein R⁵ represents a C₁₈ alkyl group.

8. A composition as claimed in claim 1, wherein X² represents

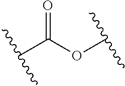 [SMILES: *OC(*)=O]

9. A composition as claimed in claim 1, wherein R⁶ represents a C₁₅-C₂₂ alkanediyl group, preferably wherein R⁶ represents a C₁₈ to C₂₀ alkanediyl group.

10. A composition as claimed in claim 1, wherein R⁷ represents a C₁₇ or C₁₈ alkenyl group containing one double bond, preferably wherein X² represents 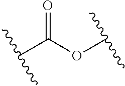 [SMILES: *OC(*)=O]
and R⁷ represents

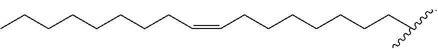 [SMILES: *CCCCCCC/C=C\CCCCCCCC]

11. A composition as claimed in claim 1, wherein the fatty acid ester of a hydroxy fatty acid of formula I is selected from the group consisting of: 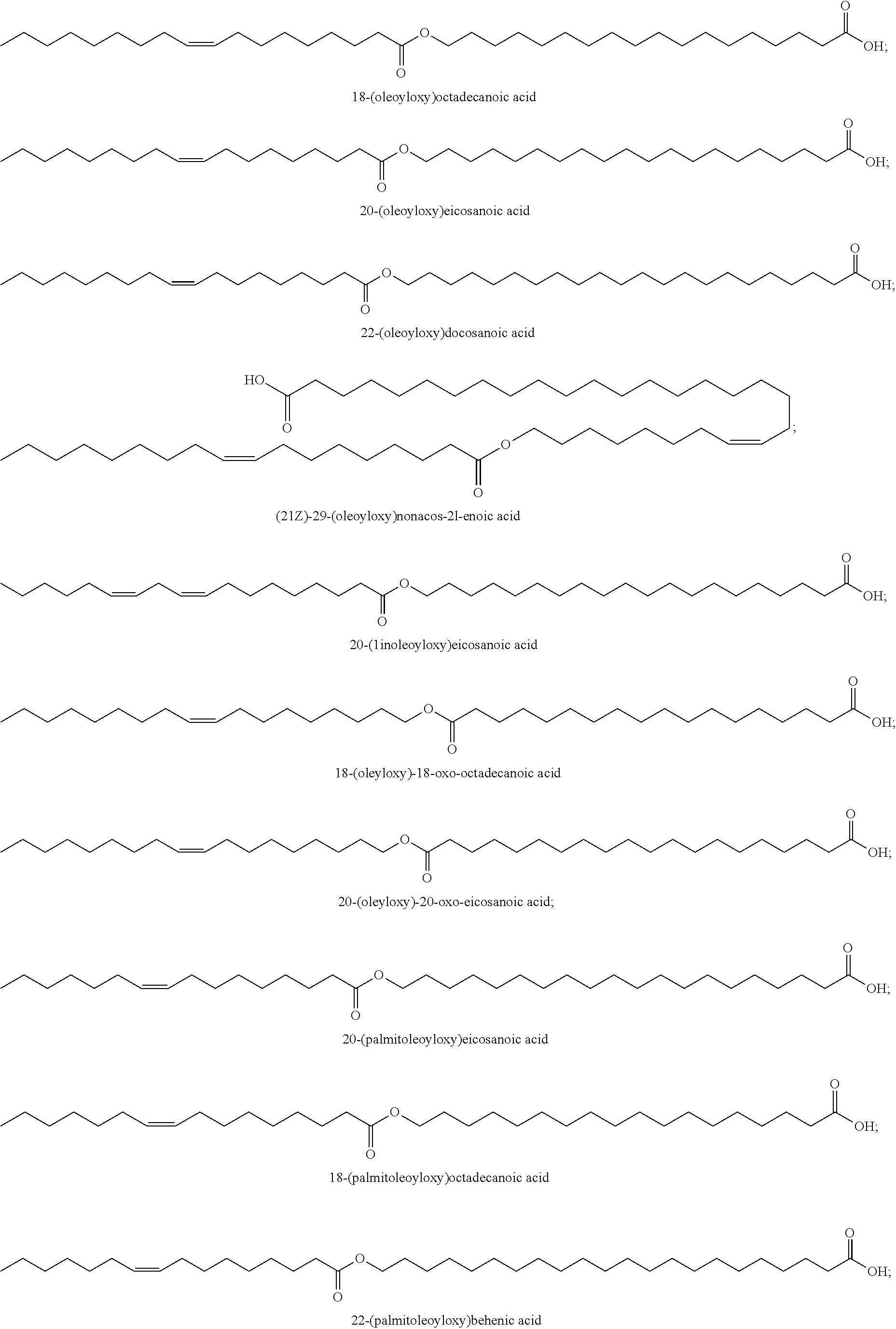 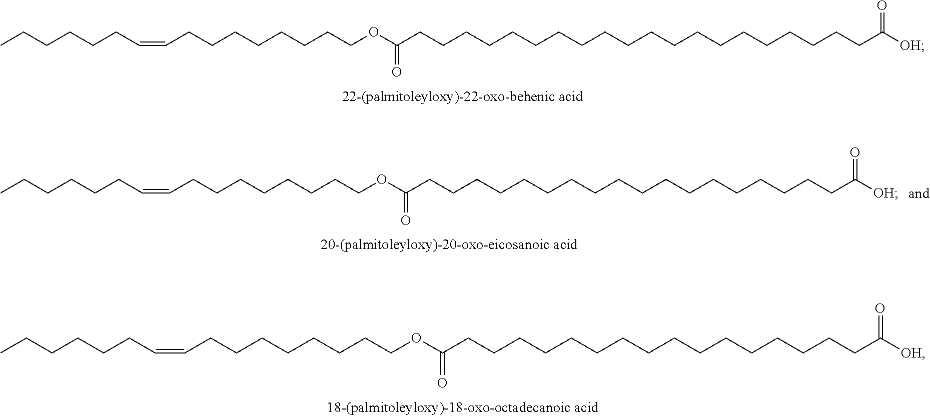 [SMILES: CCCCCC/C=C\CCCCCCCCOC(=O)CCCCCCCCCCCCCCCCCCCCC(=O)O ; CCCCCC/C=C\CCCCCCCCOC(=O)CCCCCCCCCCCCCCCCCCC(=O)O ; "and" ; CCCCCC/C=C\CCCCCCCCOC(=O)CCCCCCCCCCCCCCCCC(=O)O]
optionally wherein the fatty acid ester of a hydroxy fatty acid of formula I is selected from the group consisting of:

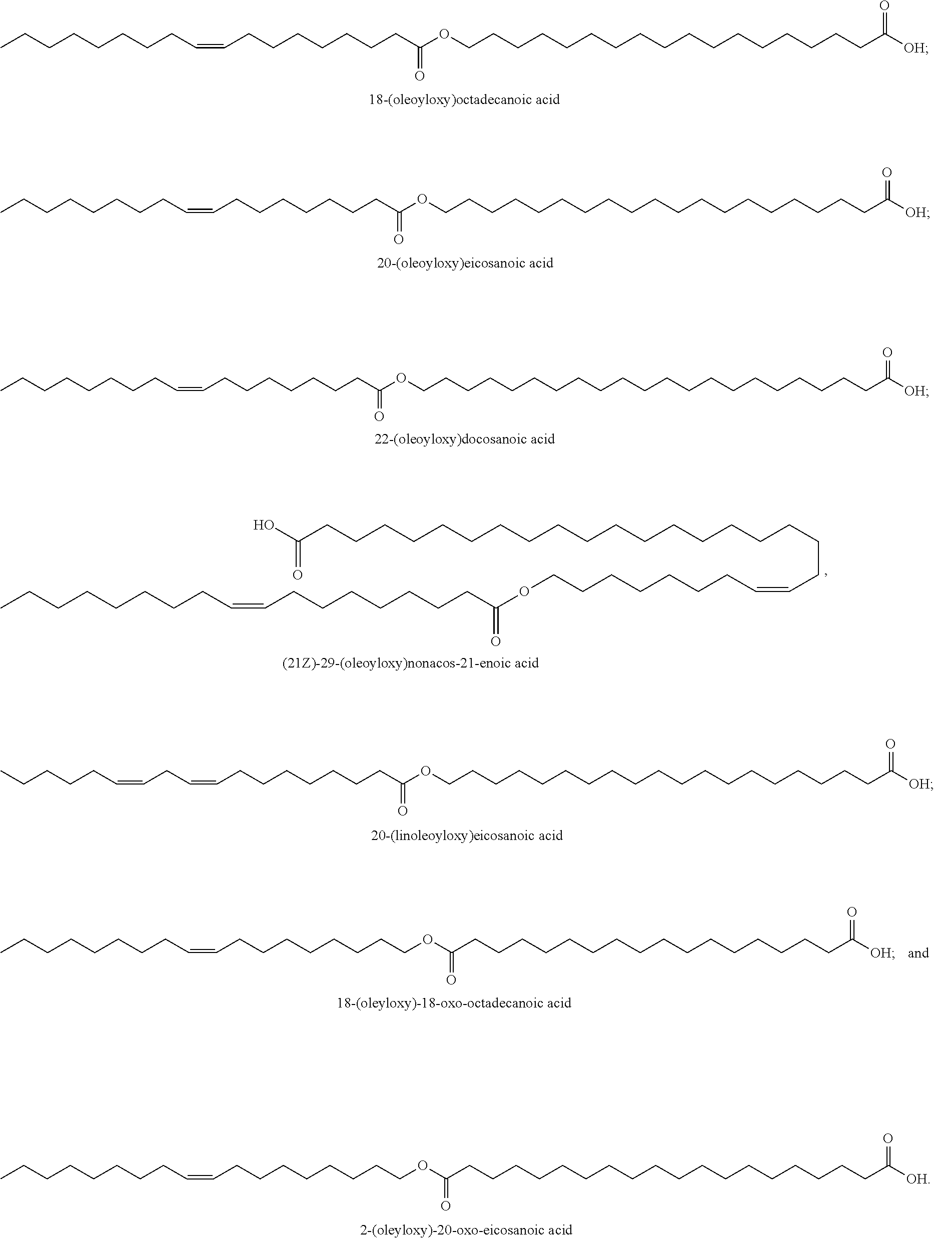

12. A composition as claimed in claim 11, wherein the fatty acid ester of a hydroxy fatty acid of formula I is selected from the group consisting of: 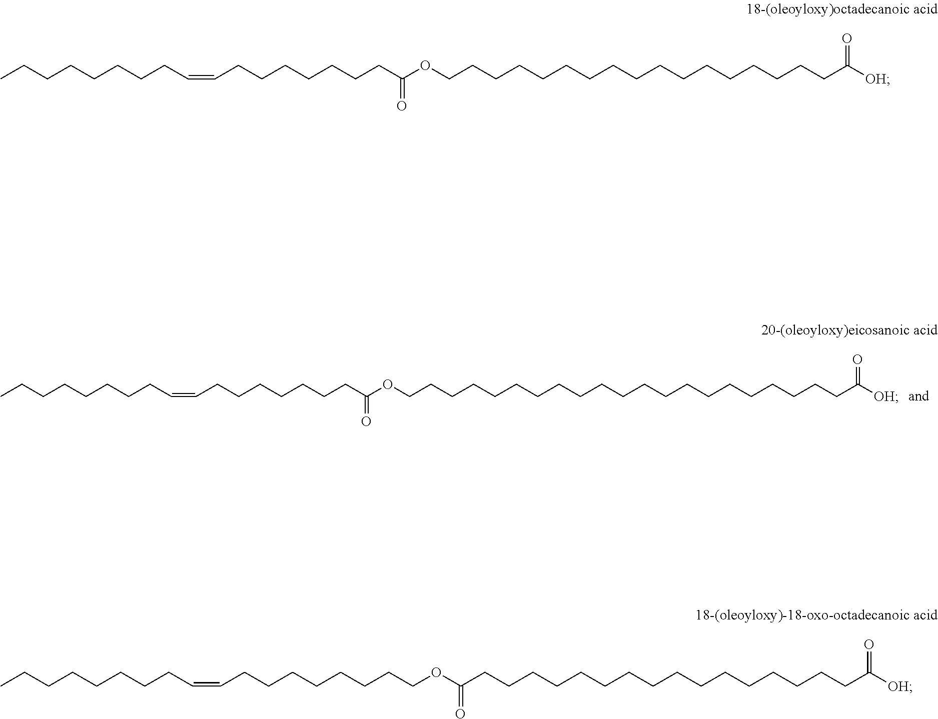 [SMILES: CCCCCCCC/C=C\CCCCCCCC(=O)OCCCCCCCCCCCCCCCCCC(=O)O ; CCCCCCCC/C=C\CCCCCCCC(=O)OCCCCCCCCCCCCCCCCCCCCCC(=O)O ; CCCCCCCC/C=C\CCCCCCCCOC(=O)CCCCCCCCCCCCCCCCC(=O)O]
preferably wherein the fatty acid ester of a hydroxy fatty acid of formula I is

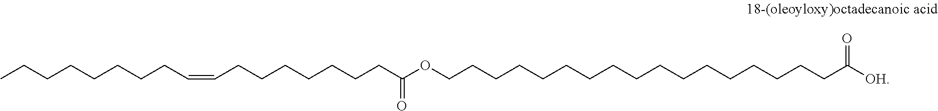 [SMILES: CCCCCCCC/C=C\CCCCCCCC(=O)OCCCCCCCCCCCCCCCCCC(=O)O]

13. A composition as claimed in claim 1, wherein the fatty acid ester of a hydroxy fatty acid of formula I is selected from the group consisting of:

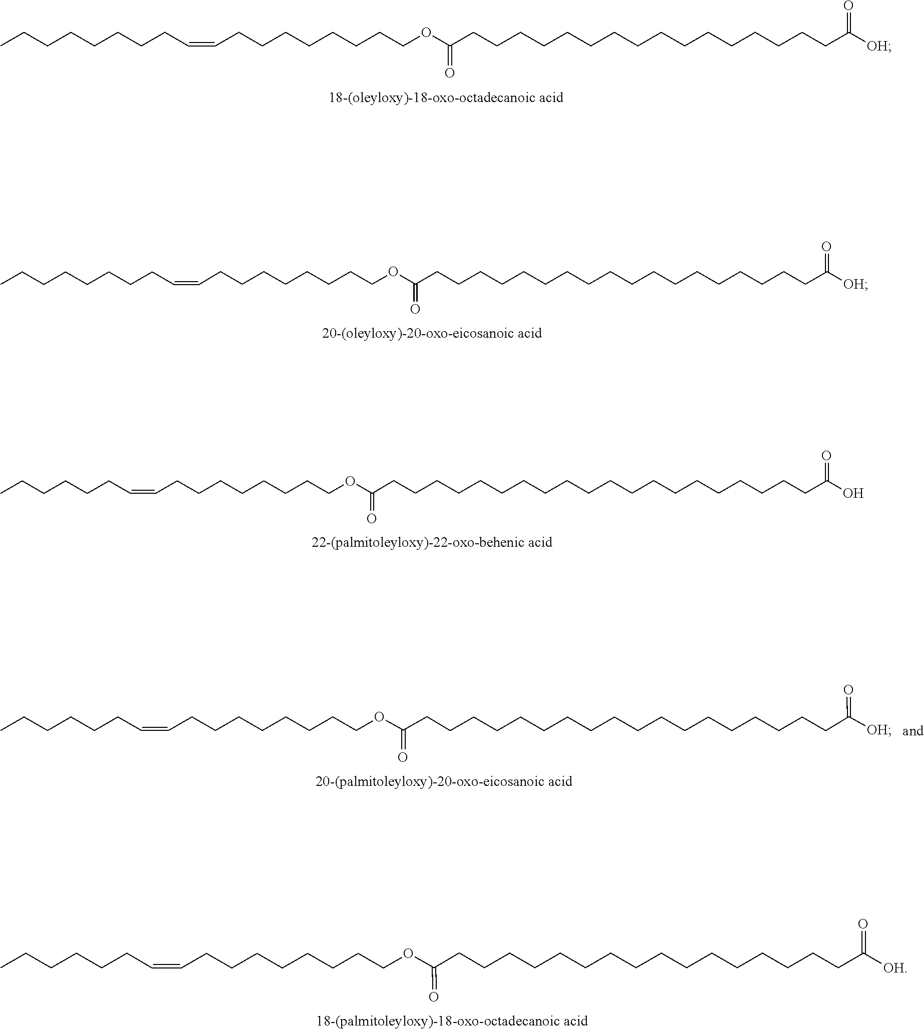 [SMILES: CCCCCCCC/C=C\CCCCCCCCOC(=O)CCCCCCCCCCCCCCCCC(=O)O ; CCCCCCCC/C=C\CCCCCCCCOC(=O)CCCCCCCCCCCCCCCCCCC(=O)O ; CCCCCC/C=C\CCCCCCCCOC(=O)CCCCCCCCCCCCCCCCCCCCC(=O)O ; CCCCCC/C=C\CCCCCCCCOC(=O)CCCCCCCCCCCCCCCCCCC(=O)O ; CCCCCC/C=C\CCCCCCCCOC(=O)CCCCCCCCCCCCCCCCC(=O)O]

14. A composition as claimed in claim 1, wherein the wax ester of formula II is selected from the group consisting of:

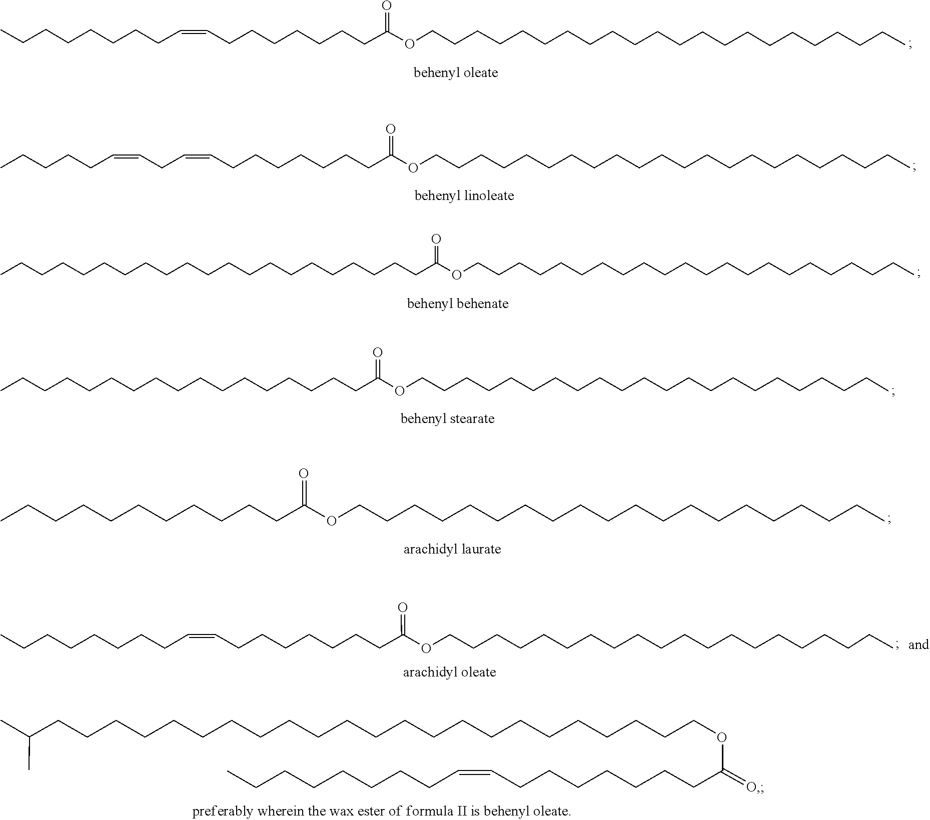

15. A composition as claimed in claim 1, wherein the fatty alcohol is of formula III and is octadecanol.

16. A composition as claimed in claim 1, wherein the lipid component is a fatty acid ester of a hydroxy fatty acid of formula I, or a pharmaceutically acceptable salt thereof, as defined in any one of the preceding claims.

17. A composition as claimed in claim 1, wherein the lipid component is a combination of a fatty acid ester of a hydroxy fatty acid of formula I, or a pharmaceutically acceptable salt thereof, and a wax ester of formula II, wherein the fatty acid ester of a hydroxy fatty acid of formula I and the wax ester of formula II are present in a molar ratio of from about 1:1 to about 1:9, such as about 1:1; optionally wherein the lipid component is a selected from the group consisting of:
a combination of 20-(oleoyloxy) eicosanoic acid, or a pharmaceutically acceptable salt thereof, and behenyl oleate;
a combination of 20-(oleoyloxy) eicosanoic acid, or a pharmaceutically acceptable salt thereof, and arachidyl laurate;
a combination of 18-(oleoyloxy) octadecanoic acid, or a pharmaceutically acceptable salt thereof, and behenyl oleate;
a combination of 18-(oleoyloxy) octadecanoic acid, or pharmaceutically acceptable salt thereof, and behenyl linoleate;
a combination of 18-(oleoyloxy) octadecanoic acid, or a pharmaceutically acceptable salt thereof, and behenyl behenate;
a combination of 18-(oleoyloxy) octadecanoic acid, or a pharmaceutically acceptable salt thereof, and behenyl stearate;
a combination of 18-(oleoyloxy) octadecanoic acid, or a pharmaceutically acceptable salt thereof, and arachidyl laurate;
a combination of 18-(oleyloxy)-18-oxo-octadecanoic acid, or a pharmaceutically acceptable salt thereof, and behenyl oleate;
a combination of (21Z)-29-(oleoyloxy) nonacos-21-enoic acid, or a pharmaceutically acceptable salt thereof, and 24-methylpentacosyl oleate
wherein, for each combination, the fatty acid ester of a hydroxy fatty acid and wax ester are present in a molar ratio of about 1:1.

18. A composition as claimed in claim 17, wherein the lipid component is selected from a combination of 18-(oleoyloxy) octadecanoic acid, or a pharmaceutically acceptable salt thereof, and behenyl oleate in a 1:1 molar ratio and a combination of 20-(oleoyloxy) eicosanoic acid, or a pharmaceutically acceptable salt thereof, and behenyl oleate in a 1:1 molar ratio.

19. A composition as claimed in claim 1, wherein the phospholipid component is selected from the group consisting of 1,2-dimyristoyl-sn-glycero-3-phosphocholine (DMPC),,2-dipentadecanoyl-sn-glycero-3-phosphocholine, dipalmitoylphosphatidyl choline (DPPC), 1,2-distearoyl-sn-glycero-3-phosphocholine (DSPC), 1,2-diarachidoyl-sn-glycero-3-phosphatidylcholine (DAPC), 1,2-dioleoyl-sn-glycero-3-phosphocholine (DOPC), and mixtures thereof; optionally
wherein the phospholipid component is selected from the group consisting of 1,2-distearoyl-sn-glycero-3-phosphocholine (DSPC), 1,2-diarachidoyl-sn-glycero-3-phosphatidylcholine (DAPC), 1,2-dioleoyl-sn-glycero-3-phosphocholine (DOPC), and mixtures thereof; preferably wherein the phospholipid component is selected from the group consisting of 1,2-distearoyl-sn-glycero-3-phosphocholine (DSPC), 1,2-diarachidoyl-sn-glycero-3-phosphatidylcholine (DAPC), and mixtures thereof.

20. A composition as claimed in claim 1, wherein the lipid component is selected from
(i) a fatty acid ester of a hydroxy fatty acid of formula I, or a pharmaceutically acceptable salt thereof, as defined in any one of the preceding claims; and
(ii) a combination of a fatty acid ester of a hydroxy fatty acid of formula I, or a pharmaceutically acceptable salt thereof, and a wax ester of formula II, as defined in any of the preceding claims, wherein the fatty acid ester of a hydroxy fatty acid of formula I and the wax ester of formula II are present in a molar ratio of from about 1:1 to about 1:9, such as about 1:1; and
the phospholipid component is a phosphatidylcholine selected from the group consisting of 1,2-distearoyl-sn-glycero-3-phosphocholine (DSPC), 1,2-diarachidoyl-sn-glycero-3-phosphatidylcholine (DAPC).

21. A composition as claimed in claim 20, wherein the lipid component is selected from selected from the group consisting of:

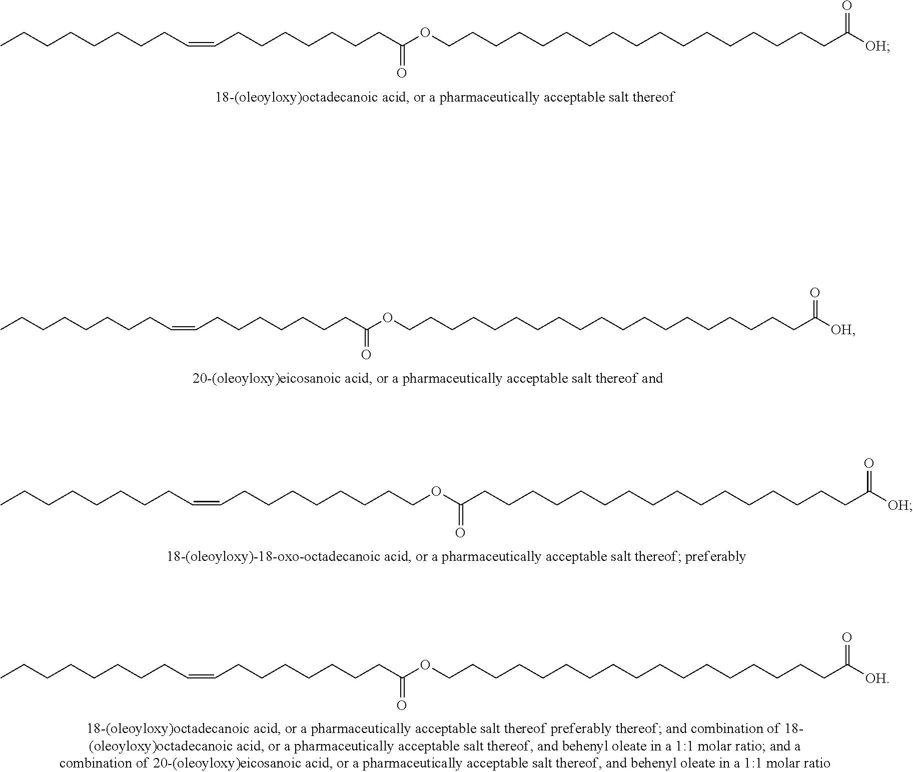 [SMILES: CCCCCCCC/C=C\CCCCCCCC(=O)OCCCCCCCCCCCCCCCCCC(=O)O ; CCCCCCCC/C=C\CCCCCCCC(=O)OCCCCCCCCCCCCCCCCCCCC(=O)O ; CCCCCCCC/C=C\CCCCCCCCOC(=O)CCCCCCCCCCCCCCCCC(=O)O ; CCCCCCCC/C=C\CCCCCCCC(=O)OCCCCCCCCCCCCCCCCCC(=O)O]

22. A composition as claimed in claim 1, wherein the lipid component and phospholipid component are present, in the composition, in a ratio of about 2:1 to about 1:10 by mass, preferably in a ratio of from about 1:1 to 1:4 by mass.

23. A composition as claimed in claim 1, wherein at least about 80% by mass of the lipid and phospholipid components are incorporated into the liposomes, optionally wherein at least about 90% by mass of the lipid and phospholipid components are incorporated into the liposomes and preferably wherein at least about 95% by mass of the lipid and phospholipid components are incorporated into the liposomes.

24. A liposome comprising a lipid component and a phospholipid component as defined in any one of the preceding claims in a ratio of from about 10:1 to about 1:10 by mass, optionally from about 2:1 to about 1:10 by mass, such as from about 1:1 to about 1:8 by mass, for example in a ratio of from about 1:1 to 1:4 by mass, such as in a ratio of about 1:1-1:3 by mass.

25. A pharmaceutical composition comprising a liposome as defined in claim 24.

26. A pharmaceutical composition as claimed in claim 1, wherein the combined amount of the lipid and phospholipid components in the composition is from about 0.1% (w/v) to about 20% (w/v) of the composition.

27. A pharmaceutical composition as claimed in claim 1, wherein lipid component of the liposome or composition, as defined in any one of the preceding claims, the is present, in the composition, in an amount of from about 0.1 (w/v) to about 10% (w/v), optionally from about 0.5% (w/v) to about 8% (w/v) or from about 0.5% (w/v) to about 5% (w/v) or from about 0.5% to about 2% (w/v)); and/or wherein the phospholipid compound, as defined in any one of the preceding claims, is present, in the composition, in an amount of from about 0.1% (w/v) to about 10% (w/v), optionally from about 0.5% (w/v) to about 8% (w/v) or from about 0.5% (w/v) to about 5% (w/v) or from about 0.5% to about 2% (w/v)).

28. A compound selected from the group consisting of
18-(oleoyloxy) octadecanoic acid,
15-(oleyloxy)-15-oxo-pentadecanoic acid
18-(oleyloxy)-18-oxo-octadecanoic acid
20-(oleyloxy)-20-oxo-eicosanoic acid,
20-(palmitoleoyloxy) eicosanoic acid; and
18-(palmitoleoyloxy) octadecanoic acid;
or a pharmaceutically acceptable salt thereof.

29. A compound selected from the group consisting of
15-(oleyloxy)-15-oxo-pentadecanoic acid,
18-(oleyloxy)-18-oxo-octadecanoic acid,
20-(oleyloxy)-20-oxo-eicosanoic acid,
22-(palmitoleyloxy)-22-oxo-behenic acid
20-(palmitoleyloxy)-20-oxo-eicosanoic acid, and
18-(palmitoleyloxy)-18-oxo-octadecanoic acid;
or a pharmaceutically acceptable salt thereof,
optionally wherein the compound is selected from the group consisting of
15-(oleyloxy)-15-oxo-pentadecanoic acid,
18-(oleyloxy)-18-oxo-octadecanoic acid, and
20-(oleyloxy)-20-oxo-eicosanoic acid, or a pharmaceutically acceptable salt thereof.

30. A pharmaceutical composition comprising a compound as defined in claim 28, or a pharmaceutically acceptable salt thereof.

31. A method of treating a subject comprising administering to said subject a pharmaceutical composition as claim in claim 1.

32. A method as claimed in claim 31, wherein method is for the treatment and/or prevention of an ocular surface disorder or allergic conjunctivitis.

33. method as claimed in claim 32, wherein the ocular surface disorder is selected from the group consisting of dry eye disease, Meibomian gland dysfunction and blepharitis.

34. The method as claimed in claim 32, wherein the method for treatment and/or prevention comprises topically administering the liposome, compound or composition to the eye surface.

35. A pharmaceutical composition comprising a compound as defined in claim 29, or a pharmaceutically acceptable salt thereof.
